SEQUENCE LISTING (3 sequences)

SEQ ID NO: 1 - protein, 9 aa, synthetic construct
GSGKGSGKG

SEQ ID NO: 2 - protein, 13 aa, synthetic construct
GSGKGSGKGSGKG

SEQ ID NO: 3 - protein, 17 aa, synthetic construct
GSGKGSGKGSGKGSGKG

(19) United States
(12) Patent Application Publication
(10) Pub. No.: US 2026/0167669 A1
(43) Pub. Date: Jun. 18, 2026

(54) COMPOUNDS, LINKER UNITS COMPRISING THE SAME, AND USES THEREOF IN TREATING DISEASES

(71) Applicant: T-E Meds Inc., Taipei City (TW)

(72) Inventors: Hsing-Mao CHU, Taipei City (TW); Chi-Jiun PENG, Taipei City (TW); Cédric GRAUFFEL, New Taipei City (TW); Tse-Wen CHANG, Taipei City (TW); Tzu-Chen LIN, Taipei City (TW); Wei-Chen LIN, Taipei City (TW)

(21) Appl. No.: 19/351,668

(22) Filed: Oct. 7, 2025

Related U.S. Application Data
(60) Provisional application No. 63/704,037, filed on Oct. 7, 2024.

(51) Int. Cl. C07K 5/093; A61K 47/68; A61P 35/00

(52) U.S. Cl. CPC C07K 5/0819; A61K 47/68031; A61K 47/68037; A61K 47/6851; A61K 47/6889; A61P 35/00

(57) ABSTRACT

Provided herein is a compound having the structure of formula (I),

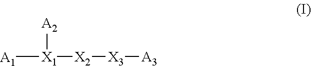

wherein X₁ is an acidic β-homo-amino acid or an acidic α-methyl-amino acid; X₂ is valine (V) or leucine (L); X₃ is citrulline, alanine (A) or lysine (L); A₁, A₂ and A₃ are independently nil or a polyethylene glycol (PEG) moiety comprising 1 to 24 repeats of ethylene glycol (EG) unit; and A₃ is nil when X₃ is not lysine. Also provided herein are linker units including the present compound of formula (I) for linking effector elements, and antibody-drug conjugates (ADCs) including the present linker units.

### REFERENCE TO A SEQUENCE LISTING

The present application is being filed along with a Sequence Listing in electronic format. The Sequence Listing is provided as a file entitled “P4417_SeqList_AF”, created Oct. 7, 2025, which is 3,535 bytes in size. The information in the electronic format of the Sequence Listing is incorporated herein by reference in its entirety.

### CROSS-REFERENCES TO RELATED APPLICATIONS

This application claims priority and the benefit of U.S. Provisional Patent Application No. 63/704,037, filed Oct. 7, 2024, the entireties of which is incorporated herein by reference

### BACKGROUND OF THE INVENTION

### 1. Field of the Invention

The present disclosure relates to the field of pharmaceuticals. More specifically, the present disclosure relates to novel linkers and their use in constructing bioconjugates for treating diseases (e.g., cancers).

### 2. Description of Related Art

Targeted drug delivery is an effective strategy to enhance therapeutic efficacy and reduce toxicity to healthy tissues compared to traditional chemotherapy. Antibody-drug conjugates (ADCs) represent a groundbreaking class of biopharmaceutical agents that combine the specificity of monoclonal antibodies (mAbs) with the potent cytotoxicity of small-molecule drugs. These mAbs are chemically linked to pharmacologically active compounds through specialized linkers, designed to remain stable in the bloodstream but release the drug upon reaching target cells. This targeted approach allows ADCs to deliver highly effective treatments directly to cancer cells or other disease-related cells, minimizing damage to healthy tissues. The clinical success of ADCs is well established, in which 15 of these drugs approved by the U.S. Food and Drug Administration (FDA) for treating both hematological cancers and solid tumors. Beyond these approvals, over 100 ADC candidates are currently in various stages of clinical trials, underscoring their growing significance in modern therapeutics.

Among the components of ADCs, the linker is the most crucial element, as it directly impacts both the efficacy and safety of the conjugates. The ideal ADC linker should remain stable during circulation but be cleavable upon entering the target cells. In the case when the linkers on ADCs are cleaved prematurely, before the ADC reaches the target cells, then off-target toxicity may occur. For example, neutropenia is a common off-target toxicity observed with ADCs conjugated to monomethyl auristatin E (MMAE) via cleavable linkers. Recent studies suggest that neutropenia results from an impact on neutrophil production from bone marrow stem cells.

Neutrophil elastase (NE), also known as leukocyte elastase, is a major protease found in the primary granules of neutrophils, where it plays a role in anti-microbial activity. NE is a serine endopeptidase with broad substrate specificity, featuring a catalytic triad of histidine, aspartate, and serine residues. During inflammation or infection, NE is secreted by activated neutrophils, where it hydrolyzes extracellular matrix proteins. While NE's protease activity is critical for normal innate immune function, the enzyme's broad substrate specificity and presence in extracellular regions or circulation usually led to undesirable toxic effects, such as neutropenia, in patients receiving drug treatments.

Recent studies have shown that the cytotoxicity of ADCs containing cleavable valine-citrulline (VC) dipeptide linkers is partly due to extracellular cleavage by proteases secreted from differentiating neutrophils. NE plays a significant role in this extracellular proteolysis of the valine-citrulline linker, highlighting a safety concern for developing stable linkers that resist NE degradation while improving the stability and therapeutic window of protein conjugates.

In view of the foregoing, there exists in the related art a need of novel linkers that improve the stability of ADCs in a patient's bloodstream and enable ADCs to effectively deliver and release drugs while mitigating off-target toxicity.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

As embodied and broadly described herein, one aspect of the present disclosure is directed to a compound having the structure of formula (I),

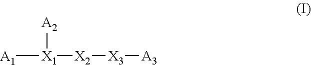

wherein,
X₁ is an acidic 3-homo-amino acid, or an acidic α-methyl-amino acid;
X₂ is valine (Val) or leucine (Leu);
X₃ is citrulline (Cit), alanine (Ala) or lysine (Lys);
A₁, A₂ and A₃ are independently nil or a polyethylene glycol (PEG) moiety comprising 1 to 24 repeats of ethylene glycol (EG) unit; and
A₃ is nil when X₃ is not lysine.

According to embodiments of the present disclosure, in formula (I), the acidic β-homo-amino acid may be β-homo-glutamic acid or β-homo-aspartic acid; and the acidic α-methyl-amino acid may be α-methyl-glutamic acid or α-methyl-aspartic acid.

According to embodiments of the present disclosure, in formula (I), A₁ is linked to X₁ via forming an amide bond with the amino (NH₂) group of the acidic β-homo-amino acid or the acidic α-methyl-amino acid; and A₂ is linked to X₁ via forming an amide bond with the δ-carboxyl group (i.e., the CO₂H group on the side chain) of the acidic β-homo-amino acid or the acidic α-methyl-amino acid. Optionally or in addition, A₃ is linked to X₃ (i.e., lysine) via forming an amide bond with the ε-amino group of the lysine.

According to some embodiments of the present disclosure, in formula (I), X₁ is β-homo-glutamic acid, X₂ is valine, X₃ is citrulline, A₁ is the PEG moiety comprising 6 repeats of EG unit, and A₂ and A₃ are independently nil.

According to some embodiments of the present disclosure, in formula (I), X₁ is β-homo-glutamic acid, X₂ is valine, X₃ is citrulline, A₁ and A₂ are independently the PEG moiety comprising 8 repeats of EG unit, and A₃ is nil.

According to some embodiments of the present disclosure, in formula (I), X₁ is β-homo-glutamic acid, X₂ is valine, X₃ is citrulline, and A₁, A₂ and A₃ are independently nil.

According to some embodiments of the present disclosure, in formula (I), X₁ is α-methyl-glutamic acid, X₂ is valine, X₃ is citrulline, and A₁, A₂ and A₃ are independently nil.

According to some embodiments of the present disclosure, in formula (I), X₁ is β-homo-glutamic acid, X₂ is valine, X₃ is lysine, A₁ is the PEG moiety comprising 6 repeats of EG unit, A₂ is nil, and A₃ the PEG moiety comprising 8 repeats of EG unit.

According to some embodiments of the present disclosure, in formula (I), X₁ is β-homo-glutamic acid, X₂ is valine, X₃ is citrulline, A₁ and A₂ are independently the PEG moiety comprising 12 repeats of EG unit, and A₃ is nil. Optionally, or in addition, in the compound of formula (I), one of the two PEG moieties further comprises a sarcosine linked to the terminus of the EG unit(s), i.e., in the form of “(EG)₁₋₁₂-sarcosine”.

The second aspect of the present disclosure pertains to a linker unit comprising a center core, 1 to 6 effector elements, and 1 to 6 linking arms respectively linking the effector elements to the center core. Specifically, the center core comprises 1 to 6 lysine (K) residues, in which any two of the K residues are adjacent to each other or are separated by a filler. According to embodiments of the present disclosure, the center core comprises a peptide of SEQ ID NOs: 1, 2 or 3. Additionally, the center core is characterized by having a conjugating group disposed at its N- or C-terminus. Preferably, the conjugating group is an azide, alkyne, tetrazine, cyclooctene or cyclooctyne group. According to some embodiments of the present disclosure, the conjugating group is linked to the first or last K residue of the center core by forming an amide bond therewith. According to alternative embodiments of the present disclosure, the center core further comprises a terminal spacer, which, depending on desired purpose, may be an N-terminal spacer linked to the N-terminus of the first K residue or a C-terminal spacer linked to the C-terminus of the last K residue; in this case, the conjugating group is linked to the terminal amino acid residue of the terminal spacer by forming an amide bond therewith. Each of the filler and the terminal spacer comprises, independently, (1) 1 to 6 non-K amino acid residues, or (2) a PEGylated amino acid having 1 to 6 repeats of ethylene glycol (EG) unit.

According to embodiments of the present disclosure, one terminus of each linking arm is linked to one of the K residues of the center core, and the other terminus of each linking arm is linked to one of the effector elements. According to embodiments of the present disclosure, each linking arm is linked to the K residue via forming an amide bond with the ε-amino group of the K residue, and is independently the compound of formula (I) or a third PEG moiety comprising 1 to 4 repeats of EG unit. Preferably, at least one of the linking arms is the compound of formula (I).

According to certain embodiments, the linker unit of the present disclosure further comprises a targeting element linked to the conjugating group of the center core. In one embodiment, the conjugating group is an azide group, and the targeting element is linked to the azide group via copper catalyzed azide-alkyne cycloaddition (CuAAC) reaction or strained-promoted azide-alkyne click chemistry (SPAAC) reaction. In another embodiment, the conjugating group is an alkyne group, and the targeting element is linked to the alkyne group via CuAAC reaction. In another embodiment, the conjugating group is a cyclooctene group, and the targeting element is linked to the cyclooctene group via inverse electron demand Diels-Alder (iEDDA) reaction. In a still another embodiment, the conjugating group is a cyclooctyne group, and the targeting element is linked to the tetrazine group via SPAAC reaction. In a further embodiment, the conjugating group is a tetrazine group, and the targeting element is linked to the tetrazine group via iEDDA reaction.

Depending on desired purpose, the targeting element may be an antibody, peptide or aptamer specific to a molecule associated with a disease. For example, the targeting element may be an antibody specific to a tumor-associated antigen (TAA); in this case, the effector element is preferably a cytotoxic drug.

Non-limiting examples of the TAA-specific antibody include, an anti-HER2 antibody, etc. Non-limiting examples of the cytotoxic drug include, acalabrutinib, exatecan, monomethyl auristatin E (MMAE), monomethyl auristatin F (MMAF), niraparib, vepdegestrant (ARV-471) and the like.

Another aspect of the present disclosure pertains to an antibody-drug conjugate (ADC). The ADC of the present disclosure in its structure comprises an antibody (e.g., an antibody specific to a TAA), and 2 to 8 of the present linker units (i.e., 2-8 linker units as described in the second and/or third aspect of the present disclosure) respectively linked to the antibody.

Also disclosed herein is a method of treating a disease (e.g., a cancer) in a subject. The method comprises administering to the subject an effective amount of the linker unit or ADC of the present disclosure.

The subject treatable with the present method is a mammal; preferably, a human.

Many of the attendant features and advantages of the present disclosure will become better understood with reference to the following detailed description considered in connection with the accompanying drawings.

### BRIEF DESCRIPTION OF THE DRAWINGS

The present description will be better understood from the following detailed description read in light of the accompanying drawings.

FIGS. 1A to 1D are schematic diagrams illustrating (1A) the MMAF-exatecan dual-drug bundle, (1B) azido-modified trastuzumab, (1C) the molecular construct, and (1D) the results of non-reducing sodium dodecyl sulfate polyacrylamide gel electrophoresis (SDS-PAGE) analysis of the construct according to certain examples of the present invention.

FIG. 2 is the result of non-reducing SDS-PAGE analysis of anti-HER2 antibody conjugated with DBCO-containing MMAE/exatecan dual drug bundle according to another working example of the present invention.

FIGS. 3A and 3B are the results of enzyme-linked immunosorbent assay (ELISA) of anti-HER2 antibody conjugated with DBCO-containing MMAF/exatecan dual drug bundle (3A) and MMAE/exatecan dual drug bundle (3B) to HER2 antigen according to one working example of the present invention.

FIG. 4 depicts the release of free exatecan or MMAF and the half-life of the anti-HER2 antibody conjugated with DBCO-containing MMAF/exatecan dual-drug bundles during a 28-day incubation period in human plasma determined by ELISA according to one working example of the present invention.

FIGS. 5A and 5B are the results of the half-lives of the anti-HER2 ADC carrying MMAF/exatecan dual drug bundles and trastuzumab deruxtecan according to one working example of the present invention.

FIG. 6 shows the results of bystander killing effect induced by the anti-HER2 ADC carrying MMAF/exatecan dual-drug bundles (“DP-ADC”), compared with T-DXd, according to one working example of the present invention.

FIGS. 7A and 7B show the in vivo anti-tumor activity in two xenograft mouse tumor models treated with vehicle, T-DXd, or the anti-HER2 ADC carrying MMAF/exatecan dual-drug bundles (“DP-ADC”), according to one working example of the present invention.

FIGS. 8A to 8C show the results of a tolerability study in healthy mice administered a single intravenous high dose of DP-ADC (40 or 60 mg/kg), according to one working example of the present invention.

### DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### I. Definitions

For convenience, certain terms employed in the specification, examples and appended claims are collected here. Unless defined otherwise, all technical and scientific terms used herein have the same meaning as commonly understood by one of the ordinary skill in the art to which this invention belongs.

Unless otherwise required by context, it will be understood that singular terms shall include plural forms of the same and plural terms shall include the singular. Also, as used herein and in the claims, the terms “at least one” and “one or more” have the same meaning and include one, two, three, or more. Furthermore, the phrases “at least one of A, B, and C”, “at least one of A, B, or C” and “at least one of A, B and/or C,” as use throughout this specification and the appended claims, are intended to cover A alone, B alone, C alone, A and B together, B and C together, A and C together, as well as A, B, and C together.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about.” Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques. Ranges can be expressed herein as from one endpoint to another endpoint or between two endpoints. All ranges disclosed herein are inclusive of the endpoints, unless specified otherwise.

As used herein, the term “β-homo amino acid” refers to a non-canonical analog of a standard amino acid where one or more methylene (—CH₂—) groups are inserted into its side chain, effectively lengthening it. The term “α-methyl amino acids” refers to amino acids where a methyl group replaces one of the hydrogen atoms attached to the alpha-carbon. This modification offers several benefits, including increased resistance to proteolytic cleavage and improved structural stability in peptides.

As used herein, the term “targeting element” refers to the portion of a linker unit that directly or indirectly binds to a target of interest (e.g., a receptor on a cell surface or a protein in a tissue) thereby facilitates the transportation of the present linker unit into the interested target. In some examples, the targeting element may direct the linker unit to the proximity of the target cell. In other cases, the targeting element specifically binds to a molecule present on the target cell surface or to a second molecule that specifically binds a molecule present on the cell surface. In some cases, the targeting element may be internalized along with the present linker unit once it is bound to the interested target, hence is relocated into the cytosol of the target cell. A targeting element may be an antibody or a ligand for a cell surface receptor, or it may be a molecule that binds such antibody or ligand, thereby indirectly targeting the present linker unit to the target site (e.g., the surface of the cell of choice). The localization of the effector (therapeutic agent) in the diseased site will be enhanced or favored with the present linker units as compared to the therapeutic without a targeting function. The localization is a matter of degree or relative proportion; it is not meant for absolute or total localization of the effector to the diseased site.

As used herein, the term “effector element” refers to the portion of a linker unit that elicits a biological activity (e.g., inducing or suppressing immune activities, exerting cytotoxic effects, inhibiting enzymes, and the like) or other functional activity (e.g., recruiting immunocytes or other therapeutic molecules), once the linker unit is directed to its target site. The “effect” can be therapeutic or diagnostic. The effector elements encompass those that bind to cells and/or extracellular immunoregulatory factors. The effector element comprises agents such as proteins, nucleic acids, lipids, carbohydrates, glycopeptides, drug moieties (both small molecule drug and biologics), compounds, elements, and isotopes, and fragments thereof.

The terms “link,” “couple,” and “conjugate” are used interchangeably to refer to any means of connecting two components either via direct linkage or via indirect linkage between two components.

The term “polypeptide” as used herein refers to a polymer having at least two amino acid residues. Typically, the polypeptide comprises amino acid residues ranging in length from 2 to about 200 residues; nonetheless, it also encompasses macromolecules that has more than 200 amino acid residues. Where an amino acid sequence is provided herein, L-, D-, or beta amino acid versions of the sequence are also contemplated. Polypeptides also include amino acid polymers in which one or more amino acid residues are an artificial chemical analogue of a corresponding naturally occurring amino acid, as well as to naturally occurring amino acid polymers. In addition, the term applies to amino acids joined by a peptide linkage or by other, “modified linkages,” e.g., where the peptide bond is replaced by an α-ester, a β-ester, a thioamide, phosphoramide, carbomate, hydroxylate, and the like.

The term “polyethylene glycol (PEG) moiety” as used herein refers to a PEG chain that comprises 1 to 24 repeating ethylene glycol (EG) units and at least one terminal carboxyl or amino group capable of forming an amide bond with the amino or carboxyl group of an amino acid residue.

As used herein, the term “terminus” with respect to a polypeptide refers to an amino acid residue at the N- or C-end of the polypeptide. Regarding a polymer, the term “terminus” refers to a constitutional unit of the polymer (e.g., the polyethylene glycol of the present disclosure) that is positioned at the end of the polymeric backbone. In the present specification and claims, the term “free terminus” is used to mean the terminal amino acid residue or constitutional unit is not chemically bound to any other molecular.

The term “antigen” or “Ag” are interchangeably used and refers to a molecule that elicits an immune response. This immune response may involve a secretory, humoral and/or cellular antigen-specific response. In the present disclosure, the term “antigen” can be any of a protein, a polypeptide (including mutants or biologically active fragments thereof), a polysaccharide, a glycoprotein, a glycolipid, a nucleic acid, or a combination thereof.

In the present specification and claims, the term “antibody” is used in the broadest sense and covers fully assembled antibodies, antibody fragments that bind with antigens, such as antigen-binding fragment (Fab/Fab′), F(ab′)2 fragment (having two antigen-binding Fab portions linked together by disulfide bonds), variable fragment (Fv), single chain variable fragment (scFv), bi-specific single-chain variable fragment (bi-scFv), nanobodies (also referred to as single-domain antibodies, sdAb), unibodies, and diabodies. The term “antibody fragment” comprises a portion of an intact antibody, preferably the antigen-binding region or variable region of the intact antibody. An antibody fragment may comprise a pair of scFv fused to the N- or C-terminal of a pair of CH2-CH3 segments derived from human 74 or 71 immunoglobulin. Typically, an “antibody” refers to a protein consisting of one or more polypeptides substantially encoded by immunoglobulin genes or fragments of immunoglobulin genes. The well-known immunoglobulin genes include the kappa, lambda, alpha, gamma, delta, epsilon, and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively. A typical immunoglobulin (antibody) structural unit is known to comprise a tetramer. Each tetramer is composed of two identical pairs of polypeptide chains, with each pair having one “light” chain (about 25 kDa) and one “heavy” chain (about 50-70 kDa). the N-terminus of each chain defines a variable region of about 100 to 110 or more amino acids primarily responsible for antigen recognition. The terms variable light chain (VL) and variable heavy chain (VH) refer to these light and heavy chains, respectively. According to embodiments of the present disclosure, the antibody fragment can be produced by modifying the nature antibody or by de novo synthesis using recombinant DNA methodologies. In certain embodiments of the present disclosure, the antibody and/or antibody fragment can be bispecific, and can be in various configurations. For example, bispecific antibodies may comprise two different antigen binding sites (variable regions). In various embodiments, bispecific antibodies can be produced by hybridoma technique or recombinant DNA technique. In certain embodiments, bispecific antibodies have binding specificities for at least two different epitopes. In many of the molecular configurations that employ antibody fragments, the antibody fragments may be substituted for antibody mimetics, which bind to the same antigenic components as the antibody fragments. Antibody mimetics include anticalins, DARPins, affibodies, filomers, ankyrins, avimers, and others.

The term “specifically binds” as used herein, refers to the ability of an antibody or an antigen-binding fragment thereof, to bind to an antigen with a dissociation constant (Kd) of no more than about 1×10⁻⁶ M, 1×10⁻⁷ M, 1×10⁻⁸ M, 1×10⁻⁹ M, 1×10⁻¹⁰ M, 1×10⁻¹¹ M, 1×10⁻¹² M, and/or to bind to an antigen with an affinity that is at least two-folds greater than its affinity to a nonspecific antigen.

The term “treatment” or “treating” as used herein includes preventative (e.g., prophylactic), curative or palliative treatment. In particular, the term “treating” as used herein refers to the application or administration of the present linker unit or ADC to a subject, who has a medical condition a symptom associated with the medical condition, a disease or disorder secondary to the medical condition, or a predisposition toward the medical condition, with the purpose to partially or completely alleviate, ameliorate, relieve, delay onset of, inhibit progression of, reduce severity of, and/or reduce incidence of one or more symptoms or features of said particular disease, disorder, and/or condition. Treatment may be administered to a subject who does not exhibit signs of a disease, disorder, and/or condition, and/or to a subject who exhibits only early signs of a disease, disorder and/or condition, for the purpose of decreasing the risk of developing pathology associated with the disease, disorder and/or condition.

The term “effective amount” as used herein refers to the quantity of the present linker unit or ADC enough to yield a desired therapeutic response. An effective amount of an agent is not required to cure a disease or condition but will provide a treatment for a disease or condition such that the onset of the disease or condition is delayed, hindered or prevented, or the disease or condition symptoms are ameliorated. The effective amount may be divided into one, two, or more doses in a suitable form to be administered at one, two or more times throughout a designated time period. The specific effective or sufficient amount will vary with such factors as particular condition being treated, the physical condition of the patient (e.g., the patient's body mass, age, or gender), the type of subject being treated, the duration of the treatment, the nature of concurrent therapy (if any), and the specific formulations employed and the structure of the compounds or its derivatives. Effective amount may be expressed, for example, as the total mass of active component (e.g., in grams, milligrams or micrograms) or a ratio of mass of active component to body mass, e.g., as milligrams per kilogram (mg/kg).

The terms “application” and “administration” are used interchangeably herein to mean the application of a linker unit or an ADC of the present invention to a subject in need of a treatment thereof.

The terms “subject” is intended to mean an animal including the human species that is treatable with the linker unit, ADC, and/or method of the present invention. The term “subject” is intended to refer to both the male and female gender unless one gender is specifically indicated. Accordingly, the term “subject” comprises any mammal, which may benefit from the treatment method of the present disclosure. Examples of a “subject” include, but are not limited to, a human, rat, mouse, guinea pig, monkey, pig, goat, cow, horse, dog, cat, bird and fowl. In an exemplary embodiment, the patient is a human. The term “mammal” refers to all members of the class Mammalia, including humans, primates, domestic and farm animals, such as rabbit, pig, sheep, and cattle; as well as zoo, sports or pet animals; and rodents, such as mouse and rat. The term “non-human mammal” refers to all members of the class Mammals except human.

### II. Detail Description of the Invention

The present disclosure is based, at least in part, on the design of novel compounds, each of which may serve as a linker for linking two molecules, for example, a targeting molecule (e.g., an anti-TAA antibody) and a therapeutic molecule (e.g., a cytotoxic drug). The thus-produced conjugate is capable of specifically targeting a lesion site (e.g., a cancerous tissue) and then eliciting a therapeutic effect thereon. Compared to conventional conjugate linkers, which, as described above, is sensitive to neutrophil elastase and usually causes uncontrolled release of therapeutic drugs from the conjugates during blood circulation leading to undesirable toxic effects in patients, the present compounds are highly resistant to neutrophil elastase, ensuring that the therapeutic drugs would only be released in lesion site thereby improving the stability and therapeutic efficacy of the conjugates. Thus, the present disclosure provides different conjugates (in the form of a linker unit or an ADC) comprising the compounds, and uses thereof in treating diseases. Aspects and embodiments of the present invention are provided below.

### (i) Compounds for Linking Two Molecules

The first aspect of the present disclosure is directed to a compound having the structure of formula (I),

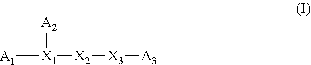

wherein
X₁ is an acidic β-homo-amino acid, or an acidic α-methyl-amino acid;
X₂ is valine (Val) or leucine (Leu);
X₃ is citrulline (Cit), alanine (Ala) or lysine (Lys);
A₁, A₂, and A₃ are independently nil or a polyethylene glycol (PEG) moiety comprising 1 to 24 repeats of ethylene glycol (EG) unit; and
A₃ is nil when X₃ is not lysine.

Non-limiting examples of the acidic β-homo-amino acid suitable for use in the present disclosure include β-homo-glutamic acid (β-homo-Glu), β-homo-aspartic acid (β-homo-Asp) and the like. Non-limiting examples of the acidic α-methyl-amino acid suitable for use in the present disclosure include α-methyl-glutamic acid (α-methyl-Glu), α-methyl-aspartic acid (α-methyl-Asp), and the like.

According to embodiments of the present disclosure, in formula (I), X₁ is β-homo-glutamic acid, X₂ is valine and X₃ is citrulline or lysine. In further embodiments, X₁ is α-methyl-glutamic acid, X₂ is valine and X₃ is citrulline.

According to some embodiments of the present disclosure, A₁ is the PEG moiety comprising 1-12 repeats of EG unit and is linked to N-terminus of X₁ via forming an amide bond with its amino (—NH₂) group; A₂ is nil or the PEG moiety comprising 4-24 repeats of EG units and is linked to the side chain of X₁ via forming an amide bond with its δ-carboxyl group; and A₃ is nil or the PEG moiety comprising 4-12 repeats of EG unit and is linked to the ε-amino group of the lysine residue. In some embodiments, A₁ has the structure of —CO—CH₂O(CH₂CH₂O)₁₋₁₂—CH₂COOH, while A₂ is in a linear form and has the structure of —NH—(CH₂CH₂O)₄₋₂₄—CH₃ or is in a branched form and has the structure of —N((CH₂CH₂O)₁₋₁₂—CH₃)₂. Optionally, A₁ may further comprise a sarcosine residue disposed at the terminus of the EG unit(s) thereby linking the EG unit(s) to X₁. According to one exemplary embodiment, A₁ has the structure of —CO—CH₂O(CH₂CH₂O)₆—CH₂CO—NHCH₃—CH₂CO₂H, A₂ is nil and A₃ has the structure of —CO((CH₂CH₂O)₈—CH₃).

In some examples of the present disclosure, in formula (I), X₁ is β-homo-glutamic acid, X₂ is valine, X₃ is citrulline, A₁ is the PEG moiety comprising 6 repeats of EG unit, and A₂ and A₃ are independently nil, and the compound has the structure of β-homo-Glu(EG₆)-Val-Cit (hereafter “Peptide 1” or “compound 17”). In other examples of the present disclosure, in formula (I), X₁ is β-homo-glutamic acid, X₂ is valine, X₃ is citrulline, A₁ and A₂ are independently the PEG moiety comprising 8 repeats of EG unit, and A₃ is nil, and the compound has the structure of 3-homo-Glu(EG₈)₂-Val-Cit (hereafter “Peptide 2” or “compound 18”). In further examples of the present disclosure, in formula (I), X₁ is β-homo-glutamic acid, X₂ is valine, X₃ is citrulline, and A₁, A₂ and A₃ are independently nil, and the compound has the structure of β-homo-Glu-Val-Cit (hereafter “Peptide 3”). In further examples of the present disclosure, in formula (I), X₁ is α-methyl-glutamic acid, X₂ is valine, X₃ is citrulline, and A₁, A₂ and A₃ are independently nil, and the compound has the structure of α-methyl-Glu-Val-Cit (hereafter “Peptide 8”). In further examples of the present disclosure, in formula (I), X₁ is β-homo-glutamic acid, X₂ is valine, X₃ is lysine, A₁ is the PEG moiety comprising 6 repeats of EG unit, A₂ is nil, and A₃ the PEG moiety comprising 8 repeats of EG unit, and the compound has the structure of β-homo-Glu(EG₆)-Val-Lys(EG₈) (hereafter “Peptide 11”). In further examples of the present disclosure, in formula (I), X₁ is β-homo-glutamic acid, X₂ is valine, X₃ is citrulline, A₁ and A₂ are independently the PEG moiety comprising 12 repeats of EG unit, and A₃ is nil, and the compound has the structure of β-homo-Glu((EG₁₂)₂)-Val-Cit (hereafter “Peptide 12”) Optionally or in addition, one of the two PEG moieties of the peptide 12 further comprises a sarcosine linked to the terminus of the EG unit(s), i.e., in the form of “(EG)₁₋₁₂-sarcosine”.

The present compound is useful in conjugating two molecules (e.g., a target molecule and an effector molecule), in which one of the molecules is linked to the N-terminus of the X₁ group, and the other of the molecules is linked to the C-terminus of the X₃ group. Optionally, the molecule is linked to the C-terminus of the X₃ group via a cleavable or self-degradable linker (e.g., p-aminobenzyl carbamate, PABC). Non-limiting examples of the cleavable or self-degradable linker suitable for use in the present invention include, but are not limited to, PABC, p-aminobenzyl alcohol (PABA), 4-nitroaniline, and amino methyl linker.

### (ii) Linker Units Comprising a Plurality of Effector Elements

The second aspect of the present disclosure pertains to a linker unit comprising a center core, 1 to 6 effector elements, and 1 to 6 linking arms respectively linking the effector elements to the center core.

Specifically, the center core is a polypeptide that has 3-120 amino acid residues in length, and comprises 1 to 6 lysine (K) residues; for example, the present center core may comprise 1, 2, 3, 4, 5, or 6 K residues. Any two of the K residues are adjacent to each other or are separated by a filler. According to some embodiments of the present disclosure, the center core further comprises a conjugating group linked to the first or last K residue of the center core by forming an amide bond therewith. Examples of the conjugating group include, but are not limited to, azide, alkyne, tetrazine, cyclooctene and cyclooctyne groups. Additionally, the center core may further comprise a terminal spacer, which, depending on intended purpose, may be an N-terminal spacer linked to the N-terminus of the first K residue or a C-terminal spacer linked to the C-terminus of the last K residue. In this case, the conjugating group is linked to the terminal amino acid residue of the terminal spacer by forming an amide bond therewith. Each of the filler and the terminal spacer comprises, independently, (1) 1 to 6 non-K amino acid residues, or (2) a PEGylated amino acid having 1 to 6 repeats of EG unit. In general, the terminal spacer or filler mentioned above may be, (1) an oligopeptide of 1-6 (i.e., 1, 2, 3, 4, 5, or 6) amino acid residues other than the K amino acid residue, or (2) a PEGylated amino acid, with EG units of 1 to 6 (i.e., having 1, 2, 3, 4, 5, or 6 EG units). Each of the non-K amino acid residues are independently selected from the group consisting of, glycine (G), aspartic acid (D), glutamic acid (E), serine (S), arginine (R), histidine (H), threonine (T), asparagine (N), glutamine (Q), proline (P), alanine (A), valine (V), isoleucine (I), leucine (L), methionine (M), phenylalanine (F), tyrosine (Y), and tryptophan (W) residues; preferably, each of the non-K amino acid residues are independently selected from the group consisting of, G, S, R, H, T, N, Q, P, A, V, I, L, M, F, Y, and W residues; more preferably, each of the non-K amino acid residues are independently G and/or S residues.

According to some embodiments of the present disclosure, the center core has two K residues separated by a filler of three non-K residues (“GSG”), an N-terminal spacer (“GSG”) linked to the N-terminus of the first K residue and a C-terminal spacer (“G”) linked to the C-terminus of the last K residue, and a conjugating group (“dibenzocyclooctyne” or DBCO) respectively linked to the terminal glycine residues of the N-terminal spacer by forming an amide bond therewith, and has the structure of DBCO-GSGKGSGKG (SEQ ID NO: 1) (hereafter “peptide 13”). Optionally, the conjugating group in peptide 13 is replaced by bicyclononyne (BCN) group and the center core has the structure of BCN-GSGKGSGKG (SEQ ID NO: 1) (hereafter “peptide 14”). Still optionally, the terminal glycine residue of the N-terminal spacer in peptide 14 is a PEGylated glycine with 2 repeats of EG unit, and the center core has the structure of BCN-(EG)₂-GSGKGSGKG (SEQ ID NO: 1) (hereafter “peptide 15”).

According to other embodiments of the present disclosure, the center core has three K residues independently separated by a filler of three non-K residues (“GSG”), an N-terminal spacer (“GSG”) linked to the N-terminus of the first K residue and a C-terminal spacer (“G”) linked to the C-terminus of the last K residue, and a conjugating groups (“DBCO”) linked to the terminal glycine residues of the N-terminal spacer by forming an amide bond therewith, and has the structure of DBCO-GSGKGSGKGSGKG (SEQ ID NO: 2) (hereafter “peptide 16”).

According to further embodiments of the present disclosure, the center core has four K residues independently separated by a filler of three non-K residues (“GSG”), an N-terminal spacer having a PEGylated glycine with 2 repeats of EG unit (“(EG)₂-GSG”) linked to the N-terminus of the first K residue, a C-terminal spacer (“G”) linked to the C-terminus of the last K residue, and a conjugating group (“BCN”) linked to the terminal glycine residue of the N-terminal spacer by forming an amide bond therewith, and has the structure of BCN-(EG₂)-GSGKGSGKGSGKGSGKG (SEQ ID NO: 3) (hereafter “peptide 17”).

The effector elements are respectively linked to the K residues of the center core via the linking arms. According to some embodiments of the present disclosure, each of the linking arms is independently the compound of formula (I) described above in section (i), or a PEG moiety comprising 1 to 4 repeats of EG unit. In these embodiments, one terminus of each linking arm is linked to the K residue of the center core via forming an amide bond with the ε-amino group of the K residue, whereas the other terminus of each linking arm is linked to the effector element. In the case when the linking arm is the present compound of formula (I), the K residue of the center core is linked to the A₁ group of the compound, and the effector element is linked to the X₃ group of the compound in the presence or absence of a cleavable or self-degradable linker (e.g., PABC). In one embodiment, the effector element is linked to the X₃ group of the compound of formula (I) without a linker. In another embodiment, the effector element is linked to the X₃ group of the compound of formula (I) via a cleavable or self-degradable linker. According to one exemplary embodiment, the effector element is linked to the X₃ group of the compound of formula (I) via a PABC linker.

According to the embodiments of the present disclosure, at least one of the linking arms is the present compound of formula (I). For example, in the case when the linker unit comprises 6 linking arms, then 1-6 (i.e., 1, 2, 3, 4, 5, or 6) of the linking arms is/are the compound of formula (I), and the rest (if any) of the linking arms is/are the PEG moiety comprising 1 to 4 repeats of EG unit. In one exemplary embodiment, the linker unit comprises 2 linking arms, in which one of the linking arms is the compound of formula (I), and the other of the linking arm is the PEG moiety comprising 1 to 4 repeats of EG unit. In another exemplary embodiment, the linker unit comprises 2 linking arms, each of which is the compound of formula (I). In still another exemplary embodiment, the linker unit comprises 3 linking arms, in which two of the linking arms are the compound of formula (I), and one of the linking arms is the PEG moiety comprising 1 to 4 repeats of EG unit.

According to some embodiments of the present disclosure, the PEG moiety comprising 1 to 4 repeats of EG unit has the structure of —CO—CH₂O(CH₂CH₂O)₁₄—CH₂COOH, in which the terminal carboxy group may react with an amino group of an amino acid.

Optionally, the linker unit further comprises a target molecule linked to the conjugating group of the center core via any of the following chemical reactions:
(i) the Copper(I)-catalyzed alkyne-azide cycloaddition (CuAAC) reaction: in this case, one of the conjugating group and targeting molecule has an azide group, and the other has an alkyne group;
(ii) the inverse electron demand Diels-Alder (iEDDA) reaction: in this case, one of the conjugating group and targeting molecule has a tetrazine group, and the other has a cyclooctene group (e.g., a TCO or a norbornene group); or
(iii) the strained-promoted azide-alkyne click chemistry (SPAAC) reaction: in this case, one of the conjugating group and targeting molecule has an azide group, and the other has a cyclooctyne group (e.g., a DBCO or a BCN group).

According to various embodiments of the present disclosure, the tetrazine group is 1,2,3,4-tetrazine, 1,2,3,5-tetrazine, 1,2,4,5-tetrazine, or derivatives thereof, the cyclooctene group is a norbornene or a trans-cyclooctene (TCO) group; and the cyclooctyne group is selected from the group consisting of, dibenzocyclooctyne (DBCO), difluorinated cyclooctyne (DIFO), dibenzylcyclooctyne (DIBO), bicyclononyne (BCN), and dibenzoazacyclooctyne (DIBAC or ADIBO). According to one embodiment of the present disclosure, the tetrazine group is 6-methyl-tetrazine.

According to some exemplary embodiments of the present disclosure, the targeting element is an antibody (e.g., IgG or scFv) specific to a TAA, i.e., an antigen associated with and/or overexpressed on tumor or cancerous tissue. Illustrative examples of the TAA associated with and/or overexpressed on a diffused tumor include, but are not limited to, CD5, CD19, CD20, CD22, CD23, CD27, CD30, CD33, CD34, CD37, CD38, CD43, CD72a, CD78, CD79a, CD79b, CD86, CD134, CD137, CD138, and CD319. Illustrative examples of the TAA associated with and/or overexpressed on a solid tumor include, but are not limited to, human epidermal growth factor receptor (HER1), HER2, HER3, HER4, carbohydrate antigen 19-9 (CA 19-9), CA 125, carcinoembryonic antigen (CEA), mucin 1 (MUC 1), ganglioside GD2, melanoma-associated antigen (MAGE), prostate-specific membrane antigen (PSMA), prostate stem cell antigen (PSCA), mesothelin, mucine-related Tn, Sialyl Tn, Globo H, stage-specific embryonic antigen-4 (SSEA-4), and epithelial cell adhesion molecule (EpCAM). Alternatively, the targeting element may be an antibody (e.g., IgG or scFv) specific to a growth factor associated with tumor growth, migration and/or invasion; non-limiting examples of the growth factor include, epidermal growth factor (EGF), mutant EGF, epiregulin, heparin-binding epidermal growth factor (HB-EGF), vascular endothelial growth factor A (VEGF-A), basic fibroblast growth factor (bFGF), and hepatocyte growth factor (HGF). In these embodiments, the effector element is preferably a cytotoxic drug.

As would be appreciated, the cytotoxic drug may be any agent known to exhibit a cytotoxic effect on cells; for example, topoisomerase inhibitors (e.g., exatecan, SN38, Dxd, irinotecan, belotecan, camptothecin, AMDCPT, indenoisoquinoline, topotecan, doxorubicine, and PNU-159682), microtubule inhibitors (e.g., auristatin, mertansine, maytansine, emtansine, ravtansine, eribulin, indolino-benzodiazepine, cyclopropabenzindolone, and tubulysin), DNA synthesis inhibitors (e.g., pyrrolobenzodiazepine monomer and dimer, duocarmycin, and calicheamicin), RNA polymerase inhibitors (e.g., amanitin, verrucarin A, trichothecene T-2, roridin A, and phalloidin), transcription inhibitors (e.g., triptolide ST7464AA1 and dacinostat), BCL-X_{L} inhibitors (e.g., clezutoclax, venetoclax, WEHI539, and navitoclax), kinase inhibitors (e.g., genistein, dasatinib and staurosporine), protein degraders (e.g., MZ1, GNE-987, BRD4/VHL, BRD4/CRBN, ERa/VHL, and BRM/VHL), immune stimulants (e.g., zuvotolimod and XMT-1621), TLR7/8 agonist (e.g., CL264, UC-1V150, and T785), anti-estrogens (e.g., tamoxifen, raloxifene, and megestrol), LHRH agonists (e.g., goscrclin and leuprolide), anti-androgens (e.g., flutamide and bicalutamide), photodynamic therapies (e.g., vertoporfin, phthalocyanine, photosensitizer Pc4, and demethoxy-hypocrellin A), nitrogen mustards (e.g., cyclophosphamide, ifosfamide, trofosfamide, chlorambucil, estramustine, and melphalan), nitrosoureas (e.g., carmustine and lomustine), alkylsulphonates (e.g., busulfan and treosulfan), triazenes (e.g., dacarbazine and temozolomide), platinum containing compounds (e.g., cisplatin, carboplatin, and oxaliplatin), vinca alkaloids (e.g., vincristine, vinblastine, vindesine, and vinorelbine), taxoids (e.g., paclitaxel, docetaxeal, taxane, and taxol), epipodophyllins (e.g., etoposide, etoposide phosphate, teniposide, topotecan, 9-aminocamptothecin, camptoirinotecan, crisnatol, and mytomycin C), anti-metabolites, DIFR inhibitors (e.g., methotrexate, dichloromethotrexate, trimetrexate, and edatrexate), IMP dehydrogenase inhibitors (e.g., mycophenolic acid, tiazofurin, ribavirin, and EICAR), ribonuclotide reductase inhibitors (e.g., hydroxyurea and deferoxamine), uracil analogs (e.g., 5-fluorouracil (5-FU), floxuridine, doxifluridine, ratitrexed, tegafur-uracil, and capecitabine), cytosine analogs (e.g., cytarabine (ara C), cytosine arabinoside, and fludarabine), purine analogs (e.g., mercaptopurine and thioguanine), vitamin D3 analogs (e.g., EB 1089, CB 1093, and KH 1060), isoprenylation inhibitors (e.g., lovastatin), dopaminergic neurotoxins (e.g., 1-methyl-4-phenylpyridinium ion), cell cycle inhibitors (e.g., staurosporine), actinomycin (e.g., actinomycin D and dactinomycin), bleomycin (e.g., bleomycin A2, bleomycin B2, peplomycin), anthracycline (e.g., daunorubicin, idarubicin, epirubicin, pirarubicin, zorubicin, and mitoxantrone), MDR inhibitors (e.g., verapamil), Ca2+ ATPase inhibitors (e.g., thapsigargin), imatinib, thalidomide, lenalidomide, pomalidomide, tyrosine kinase inhibitors (e.g., axitinib, bosutinib, cediranib, dasatinib, erlotinib, gefitinib, imatinib, lapatinib, lestaurtinib, neratinib, nilotinib, semaxanib, sunitinib, toceranib, vandetanib, vatalanib, nilotinib, sorafenib), proteasome inhibitors (e.g., bortezomib, carfilzomib, and ixazomib), mTOR inhibitors (e.g., rapamycin, temsirolimus, everolimus, and ridaforolimus), histone deacetylase (HDAC) inhibitors (e.g., vorinostat, romidepsin, panobinostat, and belinostat), adriamycin, mutamycin, mitomycin, epipodophyllotoxins, mechlorethamine, aziridines, thiotepa, alkyl sulfonate, pentostatin, busufane, cladribine, azathioprine, streptozocin, altretamine, halofuginone, epothilone, thiogaunine, temozolamide, oblimersen, gemcitabine, carminomycin, leucovorin, pemetrexed, procarbizine, prednisolone, dexamethasone, campathecin, plicamycin, aminopterin, methopterin, porfiromycin, melphalan, leurosidine, leurosine, chlorambucil, trabectedin, procarbazine, discodermolide, hexamethyl melamine, or an analog or a derivative thereof. According to one specific embodiment of the present disclosure, the cytotoxic drug is exatecan, or auristatin or a derivative thereof. In one working example, the cytotoxic drug is monomethyl auristatin E (MMAE). In another working example, the cytotoxic drug is monomethyl auristatin F (MMAF).

As could be appreciated, the number of the effector elements carried by the present linker unit is mainly determined by the number of the K residues comprised in the center core (and thus, the number of the linking arms). Accordingly, one of ordinary skill in the art may adjust the number of the effector elements of the linker unit via altering the number of the K residues to achieve desired therapeutic effects.

(ii-1) Linker Units Comprising One Effector Element

According to some preferred embodiments of the present disclosure, the linker unit comprises a center core, one effector elements, and one linking arm linking the effector element to the center core, in which the linking arm is the present compound of formula (I) (e.g., peptide 14).

In general, the structure of this linker unit is quite similar to that of the linker unit described above, except that the center core of this linker unit only has one K residue, rather than 2-6 K residues. In addition to the center core, this linker unit further comprises a conjugating group (e.g., an azide, alkyne, tetrazine, cyclooctene and cyclooctyne group). In some cases, the conjugating group is linked to the N-terminus of the K residue by forming an amide bond therewith. In other cases, the center core further comprises a terminal spacer linked to the N-terminus of the K residue, and the conjugating group is linked to the terminal amino acid residue of the terminal spacer by forming an amide bond therewith.

The effector element is linked to the K residue of center core via the compound of formula (I). As described above, the K residue is linked to the A₁ group of the compound, and the effector element is linked to the X₃ group of the compound in the presence or absence of a cleavable or self-degradable linker (e.g., PABC). In one embodiment, the effector element is linked to the X₃ group of the compound of formula (I) without a linker. In another embodiment, the effector element is linked to the X₃ group of the compound of formula (I) via a cleavable or self-degradable linker. According to one exemplary embodiment, the effector element is linked to the X₃ group of the compound of formula (I) via a PABC linker.

Optionally, the linker unit further comprises a target molecule linked to the conjugating group of the center core via the CuAAC, SPAAC, or iEDDA reaction as described in section (ii) of the present disclosure. According to some embodiments of the present disclosure, the targeting molecule is an antibody specific to a TAA. In these embodiments, the effector element is preferably a cytotoxic drug, for example, exatecan, MMAE, or MMAF.

(iii) ADCs Comprising the Linker Units

The fourth aspect of the present disclosure provides an ADC comprising an antibody and 2 to 8 linker unis linked to the antibody. Specifically, the antibody comprises a fragment antigen-binding region (Fab region) and a fragment crystallizable region (Fc region) fused to the Fc region. The linker units are respectively linked to the Fc region of the antibody by trimannosyl ADC technology, a platform for linking drug payload(s) to a target antibody in a site-specific manner (see, for example, WO 2018/126092 A1). According to some embodiments, the antibody is first modified to conjugate with 2 to 8 (e.g., 2, 3, 4, 5, 6, 7, or 8) azide groups; the azide-modified antibody is then capable of linking to 2 to 8 (e.g., 2, 3, 4, 5, 6, 7, or 8) linker units, each of which has a cyclooctyne (e.g., DBCO) group as the conjugating group, via a SPAAC reaction occurred between the azide and cyclooctyne groups. As could be appreciated, the azide or cyclooctyne group may be alternatively substituted by different groups suitable for click chemistry, for example, an alkyne, tetrazine, or cyclooctyne group. Depending on desired purpose, the present immunoconjugate may alternatively be produced by other methods known to synthesize ADCs, for example, cysteine conjugation, lysine conjugation, disulfide re-bridging, etc. The methods for synthesizing ADCs are known in the art; hence, the detailed description is omitted herein for the sake of brevity.

According to certain exemplary embodiments, the antibody has 2 azide groups in its Fc region, and 2 DBCO-conjugated linker units (the linker units having DBCO as the conjugating group) are respectively linked to the azide groups of the antibody via SPAAC reaction; in these embodiments, the drug-to-antibody ratio (DAR) of the thus-produced immunoconjugate is about 2. According to some exemplary embodiments, the antibody has 4 azide groups in its Fc region, and 4 BCN-conjugated linker units (the linker units having BCN as the conjugating group) are respectively linked to the azide groups of the antibody via SPAAC reaction; in these embodiments, the drug-to-antibody ratio (DAR) of the thus-produced immunoconjugate is about 4. According to some exemplary embodiments, the antibody has 8 tetrazine groups in its Fc region, and 8 TCO-conjugated linker unit (the linker units having TCO as the conjugating group) are respectively linked to the tetrazine groups of the antibody via iEDDA reaction; in these embodiments, the drug-to-antibody ratio (DAR) of the thus-produced immunoconjugate is about 8.

As would be appreciated, each of linker units may be the same or different; that is, each linker unit may comprise the same or different center core, linking arm, and/or effector element.

Preferably, the antibody is specific to a TAA, and the effector element is a cytotoxic drug.

### (iv) Uses of the Present Linker Units or ADCs

Another aspect of the present disclosure is directed to a method of treating a disease (e.g., a cancer) in a subject by using the present linker unit or ADC. The method comprises administering to the subject an effective amount of the linker unit or ADC as respectively described in sections (ii)-(iii) of the present disclosure, so as to alleviate or ameliorate the symptoms associated with the disease.

Depending on intended purpose, the linker unit or ADC of the present disclosure may be administered to the subject by an appropriate route, such as intrautumoral, intraarterial, intravenous or intraperitoneal injection.

According to certain embodiments, the present linker unit or ADC is useful in treating a cancer. In these embodiments, each effect element of the center core is a cytotoxic drug (e.g., exatecan, MMAE, or MMAF), and the linker unit or ADC is administered to the subject via intrautumoral, intravenous or intraperitoneal injection.

Examples of the cancer treatable with the present method include, but are not limited to, gastric cancer, lung cancer, bladder cancer, breast cancer, pancreatic cancer, renal cancer, colorectal cancer, cervical cancer, ovarian cancer, brain tumor, prostate cancer, hepatocellular carcinoma, melanoma, esophageal carcinoma, multiple myeloma, head and neck squamous cell carcinoma, or a combination thereof.

Basically, the subject treatable with the present method is a mammal, for example, human, mouse, rat, guinea pig, hamster, monkey, swine, dog, cat, horse, sheep, goat, cow, and rabbit. Preferably, the subject is a human.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent.

### EXAMPLES

### Example 1: Syntheses of Compound 17 and Compound 18

In this example, peptide 1 (β-homo-Glu(EG₆)-Val-Cit) and peptide 2 (β-homo-Glu(EG₈)₂-Val-Cit) were designed by the present inventor and was manufactured by Shanghai WuXi AppTech Co., Ltd. (Shanghai, China).

The peptide 1 and peptide 2 were independently conjugated with the topoisomerase I inhibitor and exatecan (EXT) through a PABC group. Specifically, exatecan was attached to a β-homo-Glu[γ-carboxylic acid modified by an amino-containing EG₆ linker]-Val-Cit tripeptide and a β-homo-Glu[γ-carboxylic acid modified by an amino-containing (EG₈)₂ linker]-Val-Cit tripeptide, respectively, through the PABC group, forming a carbamate bond with exatecan. The aforementioned linkages between peptides, groups, and linkers were established through amide bond formation. The thus-synthesized molecules were then conjugated to a lysine residue via a short PEG diacid linker (CO₂H—CH₂O—CH₂CH₂O—CH₂CO₂H).

The thus-produced compound 17 had the structure of Lys-PEG linker-3-homo-Glu(EG₆)-Val-Cit-PABC-EXT, and compound 18 had the structure of Lys-PEG linker-3-homo-Glu(EG₈)₂-Val-Cit-PABC-EXT. These two compounds were used as linking arms for constructing a drug bundle with the exatecan molecule. These compounds were synthesized using the standard Fmoc-based solid phase method.

Compound 17 was synthesized by following a 16-step route provided by Shanghai WuXi AppTech Co., Ltd., progressing from compound 1 to compound 16. The stepwise standard Fmoc solid phase peptide synthesis (SPPS) procedure employs 1-[Bis(dimethylamino)methylene]-1H-1,2,3-triazolo[4,5-b]pyridinium 3-oxide hexafluorophosphate (HATU)/O-Benzotriazole-N,N,N′,N′-tetramethyl-uronium-hexafluoro-phosphate (HBTU)/N,N-diiso-propylethylamine (DIEA)/N,N-dimethylformamide (DMF) coupling chemistry, in which both HATU and HBTU served as in situ activating reagents for Fmoc protected amino acids and DIEA was used as an organic base during coupling. Na-Fmoc, side-chain protected amino acids, and 2-chlorotrityl chloride resin (CTC resin) were used in the synthesis. The following side-chain protection strategies were employed: Arg (Pbf), Trp (Boc), Thr (tBu), Lys (Dde), Tyr (tBu), Glu (tBu), Gln (Trt), Ser (tBu), His (Trt), and Cys (Trt) or Cys (Acm).

In the first step of the synthesis route of peptide 1, the compound 2 was synthesized by using the following procedure:

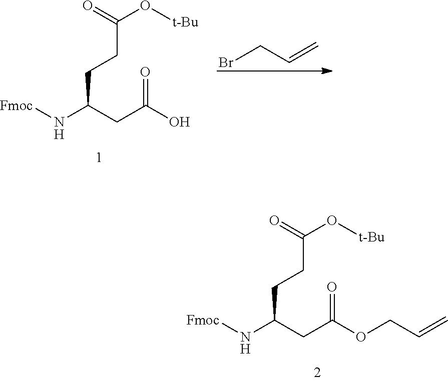

A solution of compound 1 (13.5 g, 30.7 mmol, 1.00 eq) and 3-bromoprop-1-ene (5.57 g, 46.0 mmol, 1.50 eq) in DMF (150 mL) was treated with DIEA (11.9 g, 92.1 mmol, 16.0 mL, 3.00 eq). The resulting mixture was stirred at 20° C. for 12 hours. Then, the reaction was filtered to remove the insoluble. The reaction was confirmed by liquid chromatography-mass spectrometry (LC-MS) analysis. The crude peptide was purified by preparative HPLC (TFA condition: A: 0.075% TFA in H₂O, B: ACN) to give compound 2 (11.07 g, 23.08 mmol, 75.15% yield, N/A purity) was obtained as a yellow oil and confirmed by LC-MS analysis.

The compound 3 was synthesized by using the following procedure:

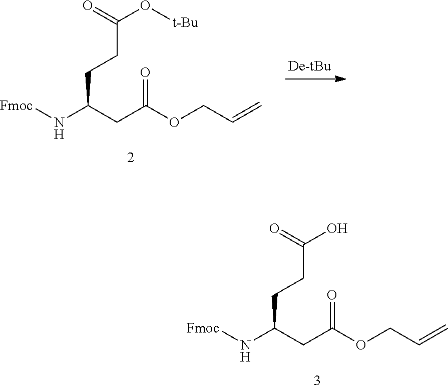

The compound 2 (11.07 g, 23.0 mmol, 1.00 eq) was dissolved in 120 mL of cleavage solution containing 57 mL DCM, 60 mL TFA, and 3.0 mL 3-mercaptopropionic acid. The resulting reaction mixture was stirred at 25° C. for 45 minutes. Then, the mixture was concentrated under reduced pressure. The reaction was confirmed by LC-MS analysis. The crude compound was purified by preparative HPLC (TFA condition: A: 0.075% TFA in H₂O, B: ACN) to give compound 3 (8.09 g, 19.1 mmol, 82.7% yield, N/A purity) was obtained as a white solid and confirmed by LC-MS analysis.

The compound 4 was synthesized by using the following procedure:

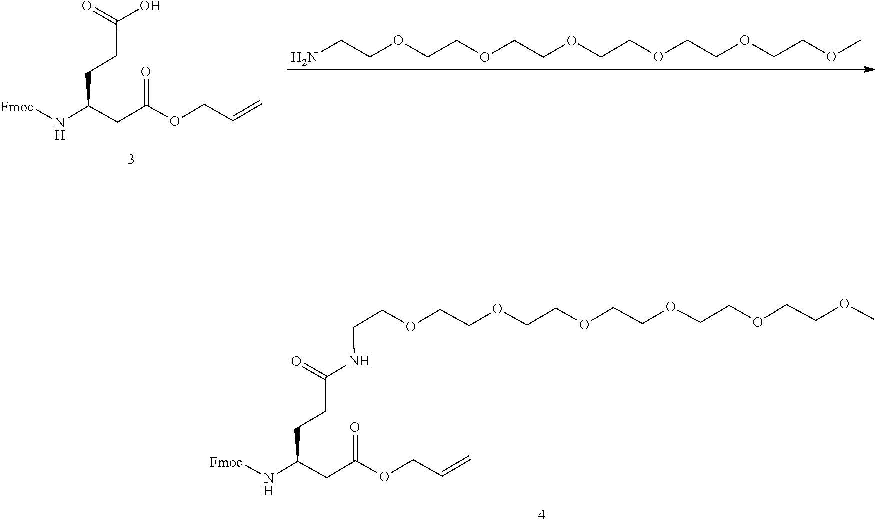

A solution of compound 3 (3.68 g, 8.69 mmol, 1.02 eq) and 2,5,8,11,14,17-hexaoxanonadecan-19-amine (2.52 g, 8.52 mmol, 1.00 eq) in DMF (45.0 mL) was treated with HOBT (2.30 g, 17.0 mmol, 2.00 eq), TBTU (5.47 g, 17.0 mmol, 2.00 eq) and DIEA (2.20 g, 17.0 mmol, 2.97 mL, 2.00 eq). The resulting mixture was stirred at 25° C. for 1 hour. Then, the reaction was filtered to remove the insoluble. The reaction was confirmed by LC-MS analysis. The crude compound was purified by preparative HPLC (TFA condition: A: 0.075% TFA in H₂O, B: ACN) to give compound 4 (3.35 g, 4.78 mmol, 56.1% yield, N/A purity) was obtained as a white solid and confirmed by LC-MS analysis.

The compound 5 was synthesized using the following procedure:

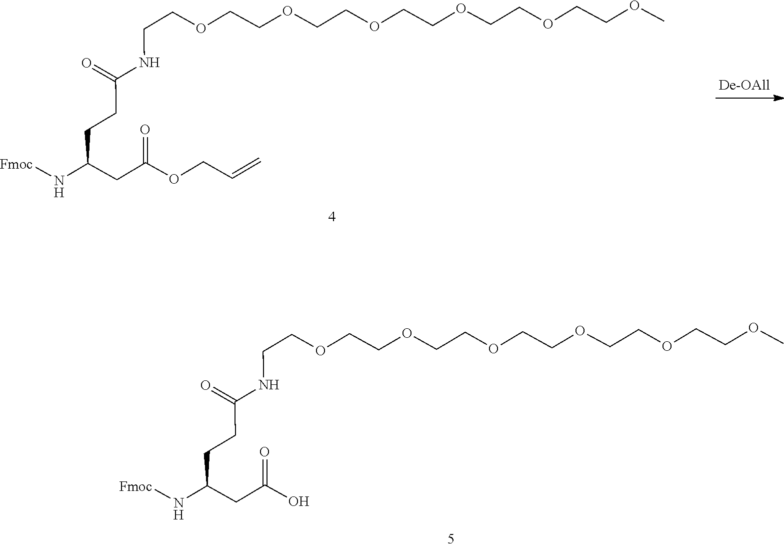

A solution of compound 4 (3.35 g, 4.78 mmol, 1.00 eq) in DCM (40.0 mL) was treated with PhSiH₃ (5.17 g, 47.8 mmol, 5.90 mL, 10.0 eq) and Pd(PPh₃)₄ (0.55 g, 478.02 mol, 0.10 eq). The resulting mixture was stirred at 25° C. for 15 minutes. The solvent was removed under vacuum by rotavapor and the crude intermediate was coupled to the 2-CTC resin. After coupling, the resin was washed 5 times with DMF (60.0 mL) and 3 times with DCM (100 mL), followed by drying under vacuum. Then, 100 mL of cleavage solution (1% TFA/99% DCM) was added to the flask containing the side chain-protected peptide resin at 20° C. and the mixture was stirred three times (5 minutes each). The solvent was removed under vacuum by rotavapor and lyophilized to give the crude compound 5 as a yellow oil (2.60 g). The crude material was confirmed by LC-MS analysis.

The compound 6 was synthesized by using the following procedure:

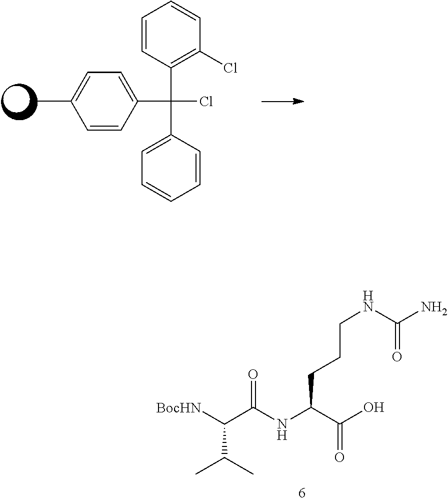 [SMILES: Cc1ccc(C(Cl)(c2ccccc2)c2ccccc2Cl)cc1 ; CC(C)[C@H](NC(=O)OC(C)(C)C)C(=O)N[C@@H](CCCNC(N)=O)C(=O)O]

A solution of 2-chlorotrityl (2-CTC) Resin (60 mmol, 1.00 eq, Sub 1.00 mmol/g) and Fmoc-Cit-OH (1.00 eq) in DCM (500 mL) was treated with DIEA (4.00 eq), the mixture was agitated with N₂ at 25° C. for 2 hours. MeOH (60.0 mL) was added to the resin and agitated with N₂ at 25° C. for 0.5 h. Then the mixture was filtered, and the resin was washed 5 times with DMF (600 mL each).

The Fmoc protecting group was removed by adding 20% piperidine in DMF (600 mL) to the resin and agitated with N₂ at 25° C. for 15 minutes. Then, the resin was washed 5 times with DMF (600 mL each) and coupling solution containing HATU (1.42 eq), Boc-Val-OH (1.50 eq) in DMF (300 mL), and DIEA (3.00 eq) was added to the filtered resin. The mixture was agitated with N₂ at 25° C. for 30 minutes. After coupling, the resin was washed 5 times with DMF (600 mL each) and 3 times with DCM (1.00 L each), followed by drying under vacuum.

Then 1.00 L of cleavage solution (20% HFIP/80% DCM) was added to the flask the side chain-protected peptide resin at 20° C. and stirred 3 times (20 minutes each). The material was concentrated under reduced pressure to give crude compound 6 as a white powder (17.5 g). The crude material was confirmed by LC-MS analysis.

The compound 7 was synthesized according to the following procedure:

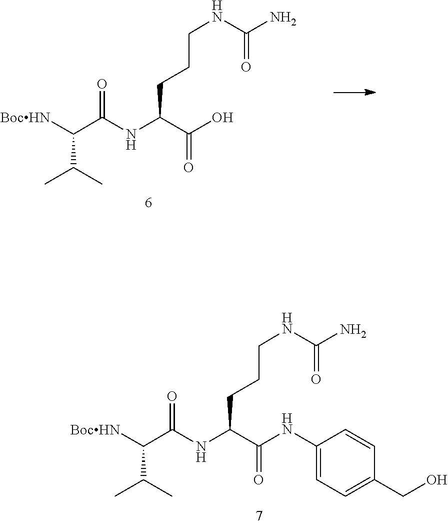

To a solution of compound 6 (7.00 g, 18.7 mmol, 1.00 eq) in DMF (400 mL) was added (4-aminophenyl)methanol (4.60 g, 37.3 mmol, 2.00 eq) in DMF (100 mL), HOBT (5.05 g, 37.3 mmol, 2.00 eq) and DIC (4.72 g, 37.3 mmol, 5.79 mL, 2.00 eq). The resulting mixture was stirred at 25° C. for 12 hours. The reaction was filtered to remove the insoluble, and the crude compound was confirmed by LC-MS analysis. The crude compound was purified by preparative HPLC (TFA condition: 30° C., A: 0.075% TFA in H₂O, B: ACN) to give compound 7 (5.34 g, 11.14 mmol, 59.56% yield, N/A purity) as a white solid. The purity of compound 7 was confirmed by LC-MS analysis.

The compound 8 was synthesized according to the following procedure:

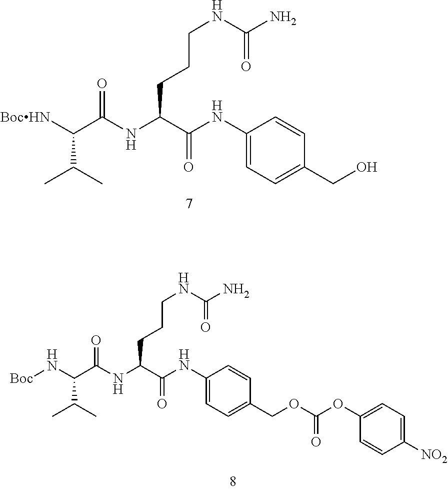

A solution of compound 7 (5.34 g, 11.1 mmol, 1.00 eq) in DMF (100 mL) was treated with DIEA (5.76 g, 44.5 mmol, 7.76 mL, 4.00 eq) and bis(4-nitrophenyl) carbonate (6.77 g, 22.2 mmol, 2.00 eq). The resulting mixture was stirred at 25° C. for 2 hours. Then, the reaction was filtered to remove the insoluble, and the crude compound was confirmed by LC-MS analysis. The crude compound was purified by preparative HPLC (TFA condition: 30° C., A: 0.075% TFA in H₂O, B: ACN) to give compound 8 (5.33 g, 8.27 mmol, 74.25% yield, N/A purity) as a white solid. The purity of compound 8 was confirmed by LC-MS analysis.

The compound 9 was synthesized according to the following procedure:

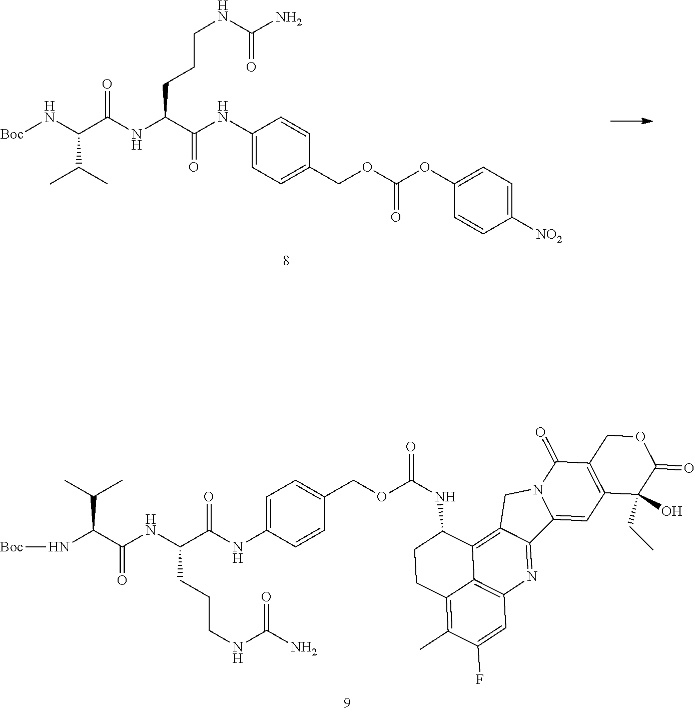 [SMILES: CC(C)[C@H](NC(=O)OC(C)(C)C)C(=O)N[C@@H](CCCNC(N)=O)C(=O)Nc1ccc(COC(=O)Oc2ccc([N+](=O)[O-])cc2)cc1 ; CC[C@@]1(O)C(=O)OCc2c1cc1n(c2=O)Cc2c-1nc1cc(F)c(C)c3c1c2[C@@H](NC(=O)OCc1ccc(NC(=O)[C@H](CCCNC(N)=O)NC(=O)[C@@H](NC(=O)OC(C)(C)C)C(C)C)cc1)CC3]

A solution of compound 8 (5.33 g, 8.27 mmol, 1.00 eq) in DMF (100 mL) was added with exatecan (EXT) (3.67 g, 8.43 mmol, 1.02 eq), HOBT (0.56 g, 4.13 mmol, 0.50 eq) and DIEA (2.14 g, 16.5 mmol, 2.88 mL, 2.00 eq). The mixture was stirred at 25° C. for 1 hour. Then, the reaction was filtered to remove the insoluble, and the reaction was confirmed by LC-MS analysis. The crude compound was purified by preparative HPLC (TFA condition: 30° C., A: 0.075% TFA in H₂O, B: ACN) to give compound 9 (5.00 g, 5.31 mmol, 64.27% yield, N/A purity) as a white solid. The compound 9 was confirmed by LC-MS analysis.

The compound 10 was synthesized according to the following procedure:

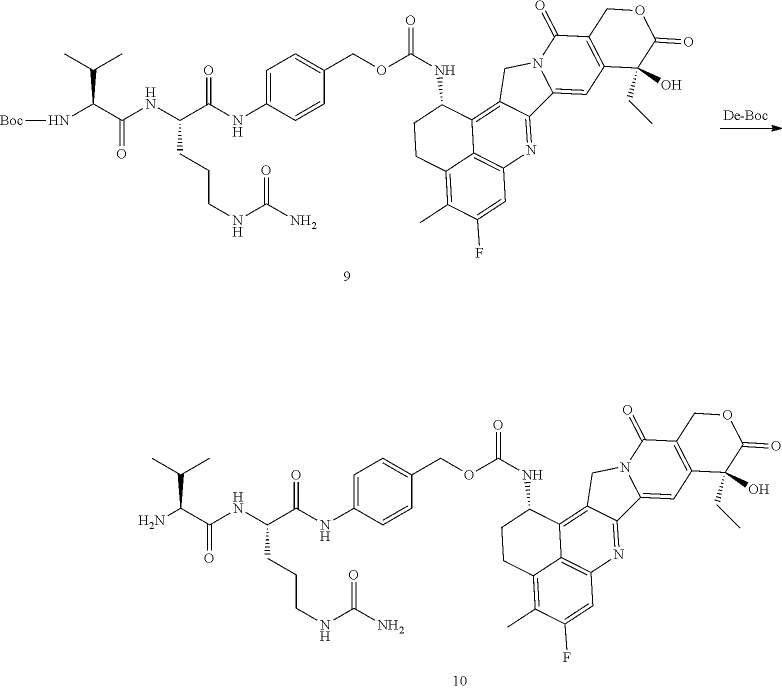 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](CCCNC(N)=O)NC(=O)[C@@H](NC(=O)OC(C)(C)C)C(C)C)cc1)CC3 ; CC[C@@]1(O)C(=O)OCc2c1cc1n(c2=O)Cc2c-1nc1cc(F)c(C)c3c1c2[C@@H](NC(=O)OCc1ccc(NC(=O)[C@H](CCCNC(N)=O)NC(=O)[C@@H](N)C(C)C)cc1)CC3]

Compound 9 (5.00 g, 5.31 mmol, 1.00 eq) was dissolved in 50.0 mL of cleavage solution containing 10.0 mL TFA and 40.0 mL DCM. The solution was stirred at 25° C. for 30 minutes. Then, the solvent was removed under vacuum by rotavapor, and the compound was precipitated with 500 mL cold isopropyl ether. The precipitates were collected by centrifugation (2 minutes at 3000 rpm) followed by two washes with 500 mL isopropyl ether each. A 5.00 g crude peptide was obtained after drying under vacuum for 2 hours and the identity was confirmed by LC-MS analysis. The crude compound was purified by preparative HPLC (TFA condition: A: 0.075% TFA in H₂O, B: ACN) to give compound 10 (3 g, 3.14 mmol, 59.13% yield, N/A purity, TFA) as a white solid. The purity of compound 10 was confirmed by LC-MS analysis.

The compound 11 was synthesized according to the following procedure:

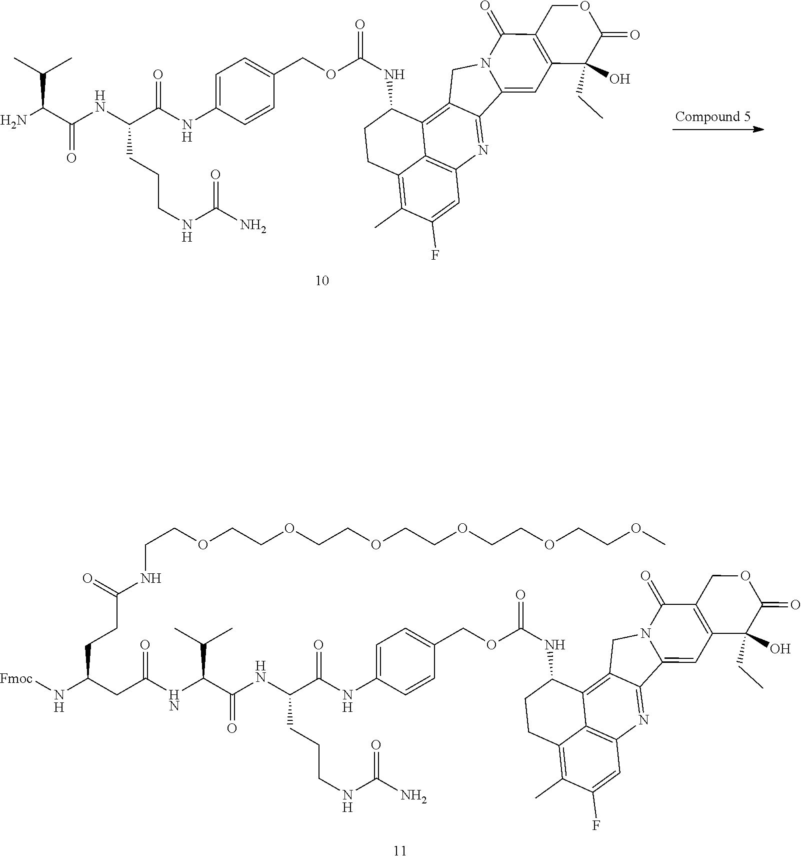

A solution of compound 10 (2.46 g, 2.57 mmol, 8.89e-1 eq, TFA) and compound 5 (1.91 g, 2.89 mmol, 1.00 eq) in DMF (45.0 mL) was treated with HATU (1.04 g, 2.75 mmol, 0.95 eq) and DIEA (0.75 g, 5.78 mmol, 1.01 mL, 2.00 eq). The resulting mixture was stirred at 25° C. for 30 minutes. Then, the reaction was filtered to remove the insoluble and the reaction was confirmed by LC-MS analysis. The reaction was purified by preparative HPLC (TFA condition: 30° C., A: 0.075% TFA in H₂O, B: ACN) to give compound 11 (3.50 g, 2.36 mmol, 81.61% yield, N/A purity) as a white solid. The purity of compound 11 was confirmed by LC-MS analysis.

The compound 12 was synthesized according to the following procedure:

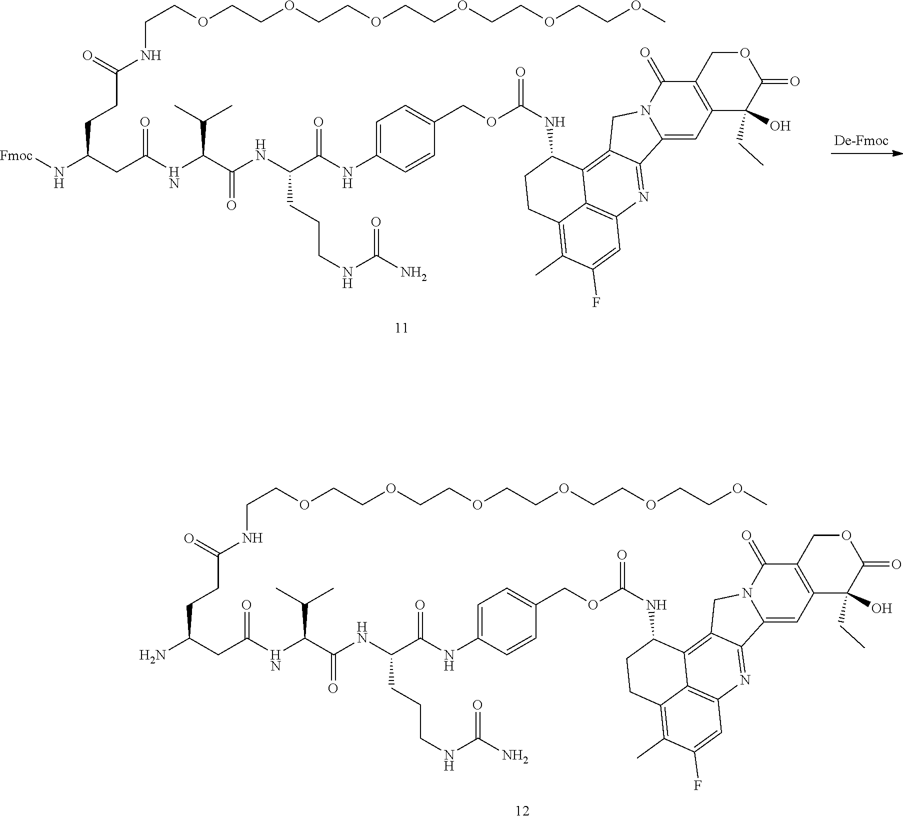

A solution of compound 11 (3.50 g, 2.36 mmol, 1.00 eq) in DMF (38.0 mL) was treated with N-ethylethanamine (1.42 g, 19.4 mmol, 2.00 mL, 8.23 eq). The resulting mixture was stirred at 25° C. for 10 minutes. Then, the reaction was filtered to remove the insoluble and the reaction was confirmed by LC-MS analysis. The reaction was purified by preparative HPLC (TFA condition: 30° C., A: 0.075% TFA in H₂O, B: ACN) to give compound 12 (2.77 g, 2.01 mmol, 85.37% yield, N/A purity, TFA) as a white solid. The purity of compound 12 was confirmed by LC-MS analysis.

The compound 14 was synthesized according to the following procedure:

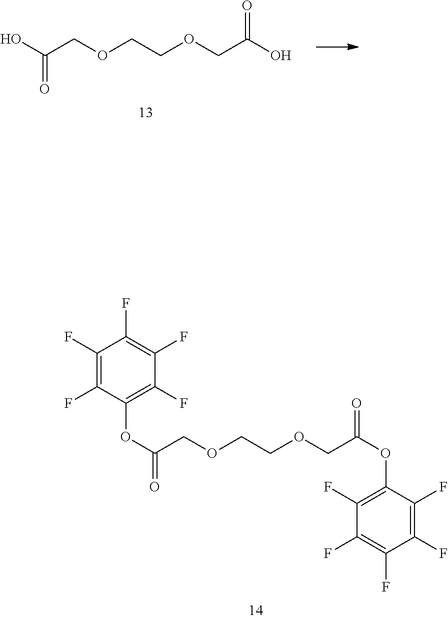 [SMILES: O=C(O)COCCOCC(=O)O ; O=C(COCCOCC(=O)Oc1c(F)c(F)c(F)c(F)c1F)Oc1c(F)c(F)c(F)c(F)c1F]

A solution of compound 13 (3.00 g, 16.8 mmol, 1.00 eq) and 2,3,4,5,6-pentafluorophenol (9.30 g, 50.5 mmol, 3.00 eq) in DMF (200 mL) was treated with EDCI (16.1 g, 84.2 mmol, 5.00 eq). The resulting mixture was stirred at 25° C. for 12 hours. Then, the reaction was filtered to remove the insoluble and the reaction was confirmed by LC-MS analysis. The reaction was purified by prep-HPLC (TFA condition: 30° C., A: 0.075% TFA in H₂O, B: ACN) to give compound 14 (5.28 g, 10.35 mmol, 61.45% yield, N/A purity) as a white solid. The purity of compound 14 was confirmed by LC-MS analysis.

The compound 15 was synthesized according to the following procedure:

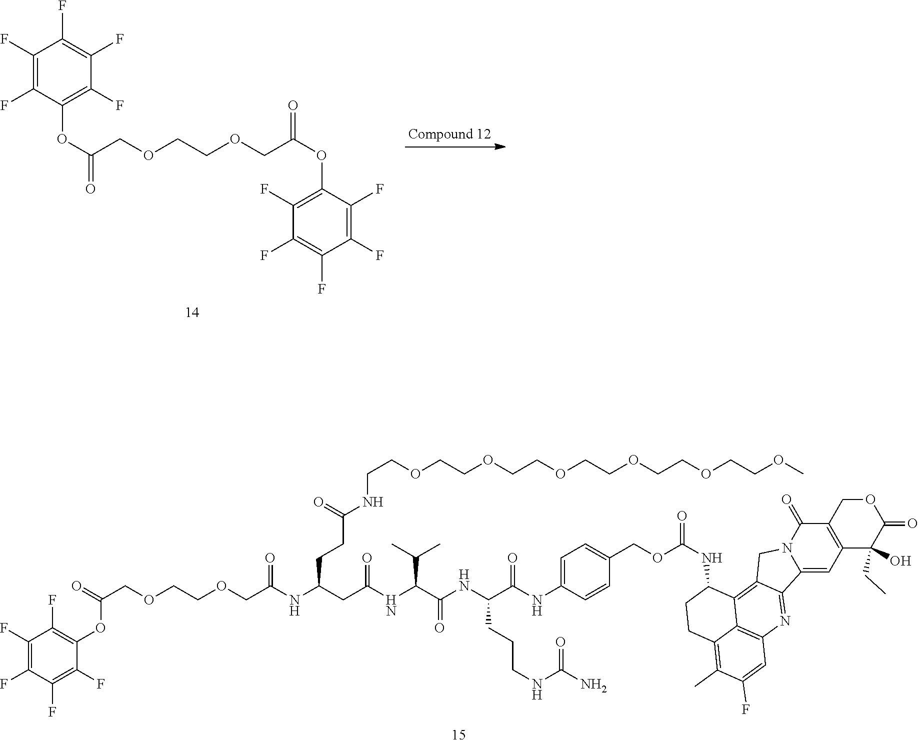 [SMILES: O=C(COCCOCC(=O)Oc1c(F)c(F)c(F)c(F)c1F)Oc1c(F)c(F)c(F)c(F)c1F ; CC[C@@]1(O)C(=O)OCc2c1cc1n(c2=O)Cc2c-1nc1cc(F)c(C)c3c1c2[C@@H](NC(=O)OCc1ccc(NC(=O)[C@H](CCCNC(N)=O)NC(=O)[C@@H](NC(=O)C[C@H](CCC(=O)NCCOCCOCCOCCOCCOCCOC)NC(=O)COCCOCC(=O)Oc2c(F)c(F)c(F)c(F)c2F)C(C)C)cc1)CC3]

A solution of compound 12 (2.77 g, 2.01 mmol, 1.00 eq, TFA salt) and compound 14 (1.05 g, 2.05 mmol, 1.02 eq) in DMF (45.0 mL) was treated with DIEA (0.26 g, 2.01 mmol, 350 L, 1.00 eq). The resulting mixture was stirred at 20° C. for 5 minutes. Then, the reaction was filtered to remove the insoluble and the reaction was confirmed by LC-MS analysis. The crude compound was purified by preparative HPLC (TFA condition: A: 0.075% TFA in H₂O, B: ACN) to give compound 15 (1.9 g, 1.20 mmol, 59.43% yield, N/A purity) as yellow oil. The purity of compound 15 was confirmed by LC-MS analysis.

The compound 16 was synthesized according to the following procedure:

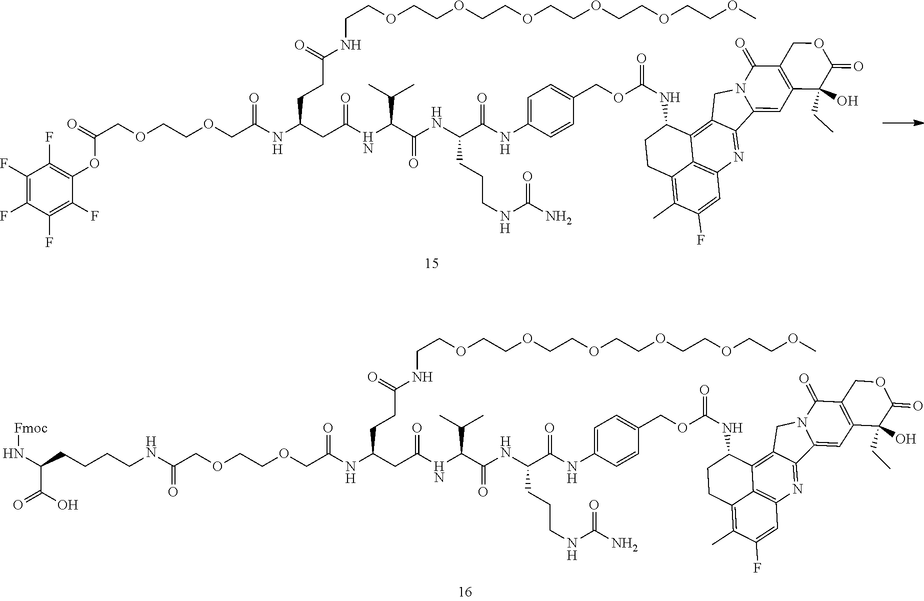

A solution of compound 15 (0.20 g, 125 mol, 1.00 eq) and Fmoc-Lys-OH HCl (46.4 mg, 125 mol, 1.00 eq) in DMF (3.00 mL) was treated with DIEA (16.2 mg, 125 mol, 21.9 L, 1.00 eq). The resulting mixture was stirred at 25° C. for 30 minutes. Then, the reaction was filtered to remove the insoluble and the reaction was confirmed by LC-MS analysis. The crude compound was purified by preparative HPLC (TFA condition: A: 0.075% TFA in H₂O, B: ACN) to give compound 16 (0.18 g, 101 mol, 80.6% yield, N/A purity) as a white solid. The compound 15 was confirmed by LC-MS analysis.

The compound 16 was synthesized according to the following procedure:

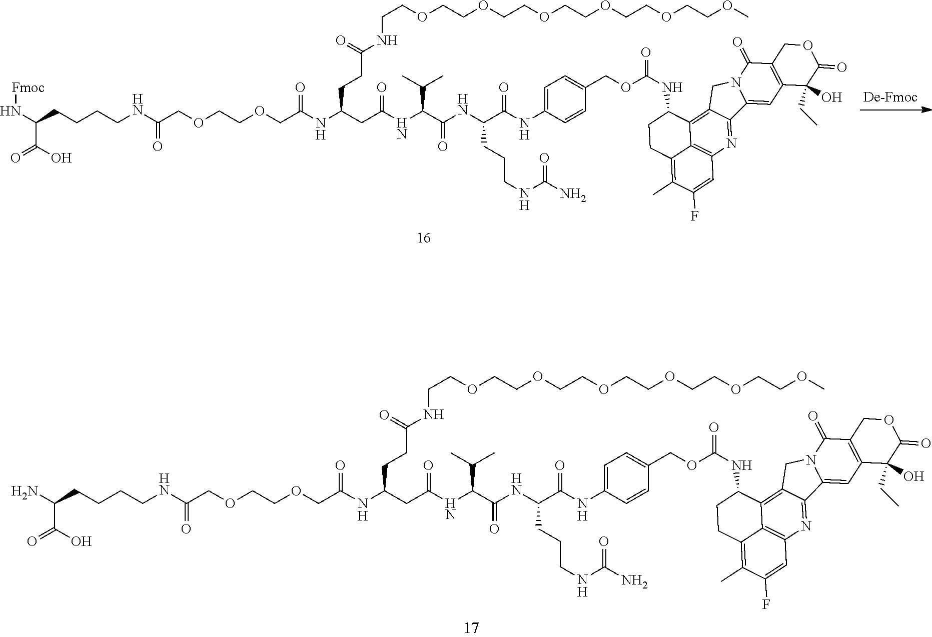

A solution of compound 16 (0.10 g, 56.4 mol, 1.00 eq) in DMF (1.42 mL) was treated with N-ethylethanamine (53.2 mg, 728 mol, 0.075 mL, 12.9 eq). The resulting mixture was stirred at 25° C. for 10 minutes. Then, the reaction was filtered to remove the insoluble and the reaction was confirmed by LC-MS analysis.

Compound 17 (i.e., peptide 1 (β-homo-Glu(EG₆)-Val-Cit)) was purified using two sequentially connected reverse-phase HPLC columns: first on a GEMINI® C18 column (150 mm×30 mm; 110 Å; 5 μm), followed by a LUNA® C18 column (120 mm×25 mm; 100 Å; 10 μm). The compound was eluted with a linear gradient of 0% to 100% ACN (solvent B) in ddH₂O (containing 0.075% trifluoroacetic acid, solvent A) over 35 minutes at a flow rate of 20.0 mL/min under a column temperature of 30° C. The purified compound 17 was used as a building block for constructing a functional bundle carrying multiple effector elements.

As illustrated below, the identification of the synthesized compound 17 was carried out by mass spectrometry ESI-MS. The synthesized compound 17 showed a molecular ion at 1550.6, corresponding to [M+H]⁺, indicating that the actual molecular weight of compound 17 is 1549.6 daltons. The reversed-phase HPLC profile of compound 17 showed the peak of the compound appearing at a retention time of 23.329 min.

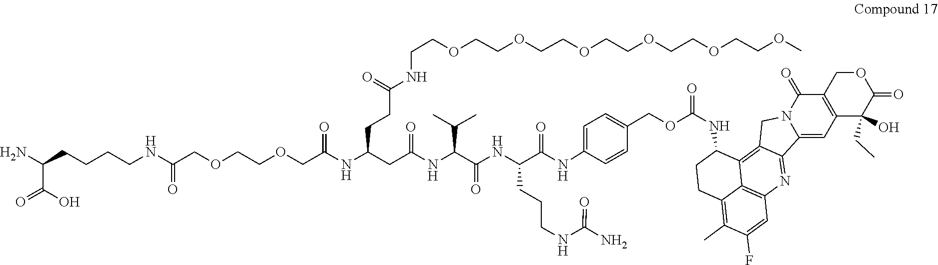 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](CCCNC(N)=O)NC(=O)[C@@H](NC(=O)C[C@H](CCC(=O)NCCOCCOCCOCCOCCOCCOC)NC(=O)COCCOCC(=O)NCCCC[C@H](N)C(=O)O)C(C)C)cc1)CC3]

The compound 18 (i.e., peptide 2 (β-homo-Glu[(EGs)₂]-Val-Cit)) was synthesized by solid-phase peptide synthesis method and purified by reverse phase HPLC as described in the compound 17. The compound 18, as illustrated below, showed a molecular ion at 2004.9872, corresponding to [M+H]⁺, indicating that the actual molecular weight of the compound is 2003.98 daltons.

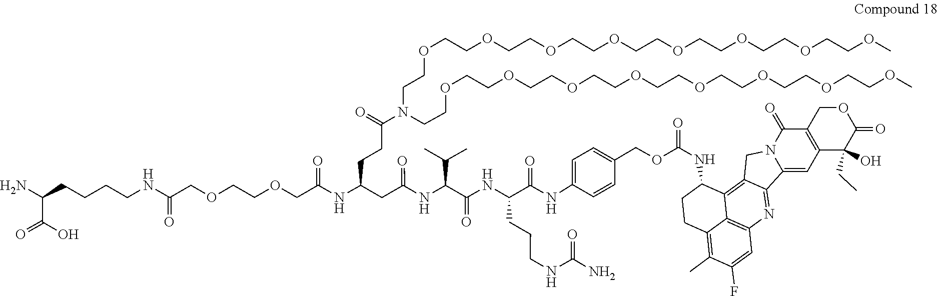 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](CCCNC(N)=O)NC(=O)[C@@H](NC(=O)C[C@H](CCC(=O)N(CCOCCOCCOCCOCCOCCOCCOCCOC)CCOCCOCCOCCOCCOCCOCCOCCOC)NC(=O)COCCOCC(=O)NCCCC[C@H](N)C(=O)O)C(C)C)cc1)CC3]

### Example 2: Synthesis of Compound 19

Peptide 3 (β-homo-Glu-Val-Cit) was synthesized by solid-phase peptide synthesis method as described in the earlier Example. The peptide 3 was then conjugated with an exatecan molecule. Specifically, the exatecan molecule was linked to a β-homo-Glu-Val-Cit tripeptide via the connection of the PABC linker, which formed an amide bond with carboxylic acid of PABC linker. The thus-synthesized molecule was then linked to a lysine residue via the connection of a short PEG diacid linker (CO₂H—CH₂O—CH₂CH₂O—CH₂CO₂H). The synthesized compound 19 had the structure of Lys-PEG linker-β-homo-Glu-Val-Cit-PABC-EXT.

The compound 19, as illustrated below, was examined using ESI-MS and showed a molecular ion at 1272,541, corresponding to [M+H]⁺, indicating that the actual molecular weight of the compound is 1271.54 daltons.

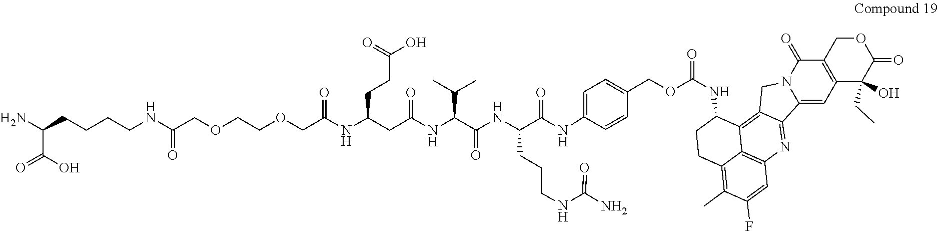 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](CCCNC(N)=O)NC(=O)[C@@H](NC(=O)C[C@H](CCC(=O)O)NC(=O)COCCOCC(=O)NCCCC[C@H](N)C(=O)O)C(C)C)cc1)CC3]

### Example 3: Synthesis of Compound 20

Peptide 4 (β-homo-Glu-Val-Gln) was synthesized by solid-phase peptide synthesis method as described in the earlier Example. The peptide 4 was conjugated with an exatecan molecule. Specifically, the exatecan molecule was linked to a β-homo-Glu-Val-Gln tripeptide via the connection of PABC linker, which formed an amide bond with carboxylic acid of PABC linker. The thus-synthesized molecule was then linked to a lysine residue via the connection of a short PEG diacid linker (CO₂H—CH₂O—CH₂CH₂O—CH₂CO₂H). The synthesized compound 20 had the structure of Lys-PEG linker-β-homo-Glu-Val-Gln-PABC-EXT.

The compound 20, as illustrated below, was examined using ESI-MS and showed a molecular ion at 1243,5204, corresponding to [M+H]⁺, indicating that the actual molecular weight of the compound is 1242.52 daltons.

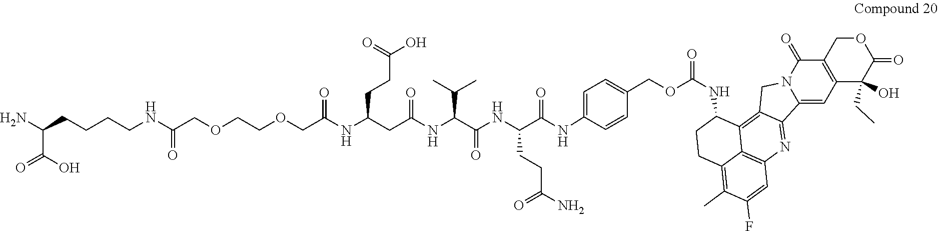 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](CCC(N)=O)NC(=O)[C@@H](NC(=O)C[C@H](CCC(=O)O)NC(=O)COCCOCC(=O)NCCCC[C@H](N)C(=O)O)C(C)C)cc1)CC3]

### Example 4: Synthesis of Compound 21

Peptide 5 (Val-Cit) was synthesized by solid-phase peptide synthesis method as described in the earlier Example. The peptide 5 was conjugated with an exatecan molecule. Specifically, the exatecan molecule was linked to a Val-Cit dipeptide via the connection of PABC linker, which formed an amide bond with carboxylic acid of PABC linker. The thus-synthesized molecule was then linked to a lysine residue via the connection of a short PEG diacid linker (CO₂H—CH₂O—CH₂CH₂O—CH₂CO₂H). the synthesized compound 21 had the structure of Lys-PEG linker-Val-Cit-PABC-EXT.

The compound 21, as illustrated below, was examined using ESI-MS and showed a molecular ion at 1129.52, corresponding to [M+H]⁺, indicating that the actual molecular weight of the compound is 1128.52 daltons.

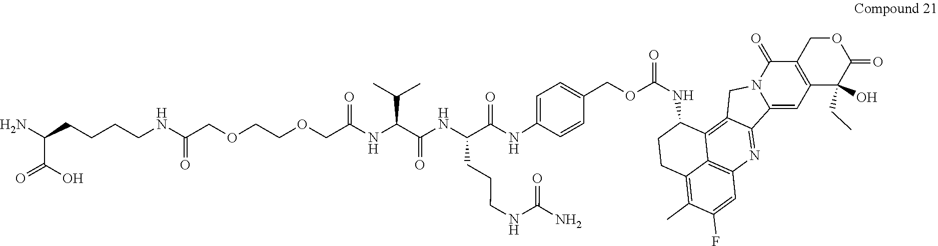 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](CCCNC(N)=O)NC(=O)[C@@H](NC(=O)COCCOCC(=O)NCCCC[C@H](N)C(=O)O)C(C)C)cc1)CC3]

### Example 5: Synthesis of Compound 22

Peptide 6 (Glu(EG₆)-Val-Cit) was synthesized by solid-phase peptide synthesis method as described in the earlier Example. The peptide 6 was conjugated with a microtubule inhibitor, named MMAE, via a PABC linker. Specifically, the MMAE molecule was linked to a Glu[gamma-acid modified by EG₆]-Val-Cit tripeptide via the connection of the PABC linker, which formed an amide bond with carboxylic acid of the PABC linker. The thus-synthesized molecule was then linked to a lysine residue via the connection of a short PEG diacid linker (CO₂H—CH₂O—CH₂CH₂O—CH₂CO₂H). The synthesized compound 22 had the structure of Lys-PEG linker-Glu(EG₆)-Val-Cit-PABC-MMAE, and was used as a linking arm for constructing an drug bundle with the MMAE molecule.

The compound 22, as illustrated below, was examined using ESI-MS. The compound 22 showed a molecular ion at 1819.07, corresponding to [M+H]⁺, indicating that the actual molecular weight of compound 6 is 1818.07 daltons. The reversed-phase HPLC profile of compound 22 showed the peak of the synthesized compound 22 appearing at a retention time of 24.442 min.

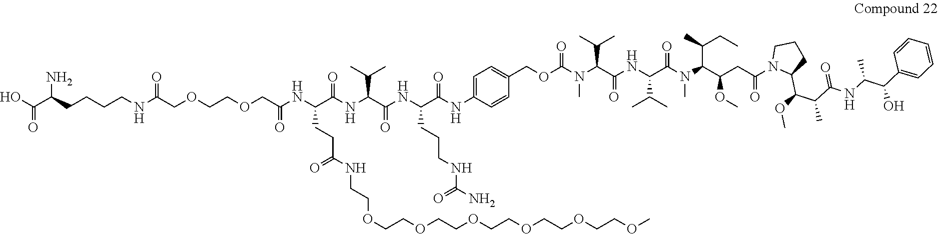 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CCC(=O)NCCOCCOCCOCCOCCOCCOC)NC(=O)COCCOCC(=O)NCCCC[C@H](N)C(=O)O)C(C)C)cc1)C(C)C]

### Example 6: Synthesis of Compound 23

Peptide 7 (Glu(EG₃)-Val-Cit) was synthesized by solid-phase peptide synthesis method as described in the earlier Example. The peptide 7 was conjugated with an MMAE molecule. Specifically, the MMAE molecule was linked to a Glu[gamma-acid modified by EG₃]-Val-Cit tripeptide via the connection of PABC linker, which formed an amide bond with carboxylic acid of PABC linker. The thus-synthesized molecule was then linked to a lysine residue via the connection of a short PEG diacid linker (CO₂H—CH₂O—CH₂CH₂O—CH₂CO₂H). The synthesized compound 23 had the structure of Lys-PEG linker-Glu(EG₃)-Val-Cit-PABC-MMAE.

The compound 23, as illustrated below, was examined using ESI-MS and showed a molecular ion at 1686.00, corresponding to [M+H]⁺, indicating that the actual molecular weight of the compound is 1685.00 daltons.

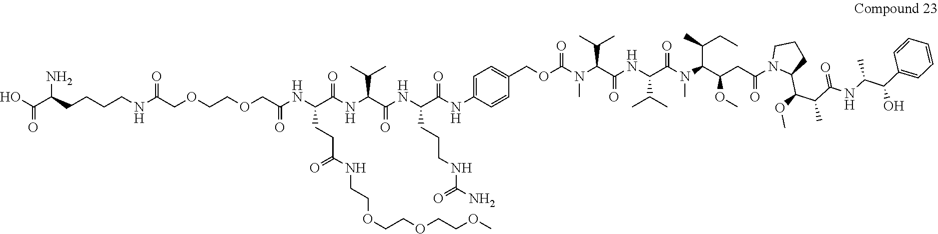 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CCC(=O)NCCOCCOCCOC)NC(=O)COCCOCC(=O)NCCCC[C@H](N)C(=O)O)C(C)C)cc1)C(C)C]

### Example 7: Synthesis of Compound 24

Compound 24 was synthesized by solid-phase peptide synthesis method as described in the earlier Example. The peptide 3 was conjugated with an MMAE molecule. Specifically, the MMAE molecule was linked to a β-homo-Glu-Val-Cit tripeptide via the connection of PABC linker, which formed an amide bond with carboxylic acid of PABC linker. Thus-synthesized molecule was then linked to a lysine residue via the connection of a short PEG diacid linker (CO₂H—CH₂O—CH₂CH₂O—CH₂CO₂H). The synthesized compound 24 had the structure of Lys-PEG linker-β-homo-Glu-Val-Cit-PABC-MMAE.

The compound 24, as illustrated below, was examined using ESI-MS and showed a molecular ion at 1554.9231, corresponding to [M+H]⁺, indicating that the actual molecular weight of the compound is 1553.9231 daltons.

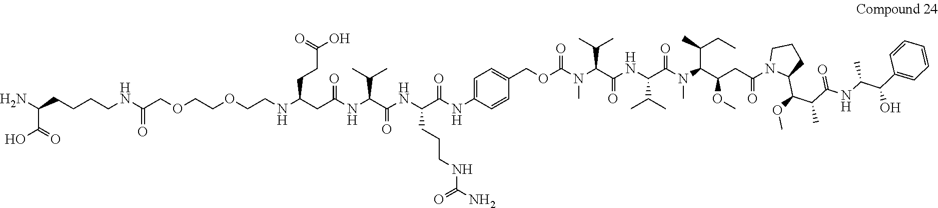 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)C[C@H](CCC(=O)O)NCCOCCOCC(=O)NCCCC[C@H](N)C(=O)O)C(C)C)cc1)C(C)C]

### Example 8: Synthesis of Compound 25

Peptide 8 (α-methyl-Glu-Val-Cit) was synthesized by solid-phase peptide synthesis method as described in the earlier Example. The peptide 8 was conjugated with an MMAE molecule. Specifically, the MMAE molecule was linked to a α-methyl-Glu-Val-Cit tripeptide via the connection of PABC linker, which formed an amide bond with carboxylic acid of PABC linker. The thus-synthesized molecule was then linked to a lysine residue via the connection of a short PEG diacid linker (CO₂H—CH₂O—CH₂CH₂O—CH₂CO₂H). The synthesized compound 25 had the structure of Lys-PEG linker-α-methyl-Glu-Val-Cit-PABC-MMAE.

The compound 25, as illustrated below, was examined using ESI-MS and showed a molecular ion at 1554.9214, corresponding to [M+H]⁺, indicating that the actual molecular weight of the compound is 1553.9214 daltons.

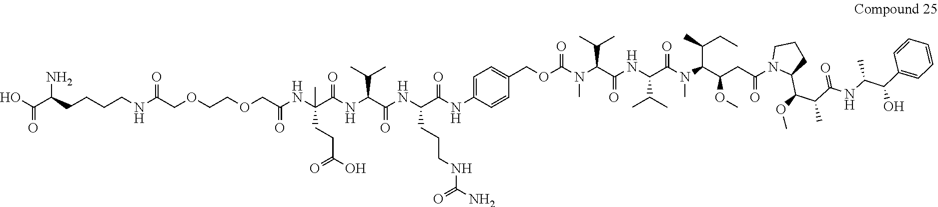 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@@](C)(CCC(=O)O)NC(=O)COCCOCC(=O)NCCCC[C@H](N)C(=O)O)C(C)C)cc1)C(C)C]

### Example 9: Synthesis of Compound 26

Peptide 9 (Glu-Val-Cit) was synthesized by solid-phase peptide synthesis method as described in the earlier Example. The peptide 9 was conjugated with an MMAE molecule. Specifically, the MMAE molecule was linked to a Glu-Val-Cit tripeptide via the connection of PABC linker, which formed an amide bond with carboxylic acid of PABC linker. The thus-synthesized molecule was then linked to a lysine residue via the connection of a short PEG linker (CO₂H—CH₂O—(CH₂CH₂O)₂—CH₂CO₂HEG₃). The synthesized compound 26 had the structure of Lys-PEG linker-Glu-Val-Cit-PABC-MMAE.

The compound 26, as illustrated below, was examined using ESI-MS and showed a molecular ion at 1585.2, corresponding to [M+H]⁺, indicating that the actual molecular weight of the compound is 1584.2 daltons.

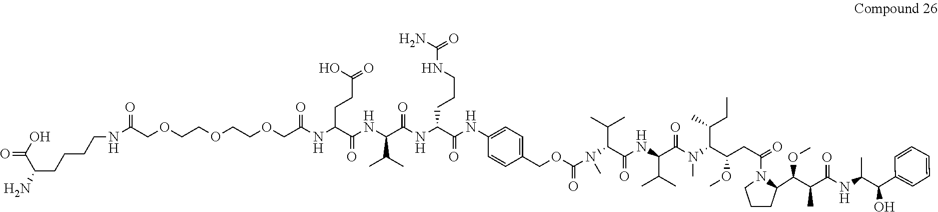 [SMILES: CC[C@@H](C)[C@H]([C@H](CC(=O)N1CCC[C@@H]1[C@@H](OC)[C@H](C)C(=O)N[C@@H](C)[C@H](O)c1ccccc1)OC)N(C)C(=O)[C@H](NC(=O)[C@@H](C(C)C)N(C)C(=O)OCc1ccc(NC(=O)[C@@H](CCCNC(N)=O)NC(=O)[C@H](NC(=O)C(CCC(=O)O)NC(=O)COCCOCCOCC(=O)NCCCC[C@H](N)C(=O)O)C(C)C)cc1)C(C)C]

### Example 10: Synthesis of Compound 27

Peptide 10 (Glu-Val-Ala) was synthesized by solid-phase peptide synthesis method as described in the earlier Example. The peptide 10 was conjugated with an MMAE molecule. Specifically, the MMAE molecule was linked to a Glu-Val-Ala tripeptide via the connection of PABC linker, which formed an amide bond with carboxylic acid of PABC linker. The thus-synthesized molecule was then linked to a lysine residue via the connection of a short PEG linker (CO₂H—CH₂O—(CH₂CH₂O)₂—CH₂CO₂H). The synthesized compound 27 had the structure of Lys-PEG linker-Glu-Val-Ala-PABC-MMAE.

The compound 27, as illustrated below, was examined using ESI-MS and showed a molecular ion at 1499.1, corresponding to [M+H]⁺, indicating that the actual molecular weight of the compound is 1498.1 daltons.

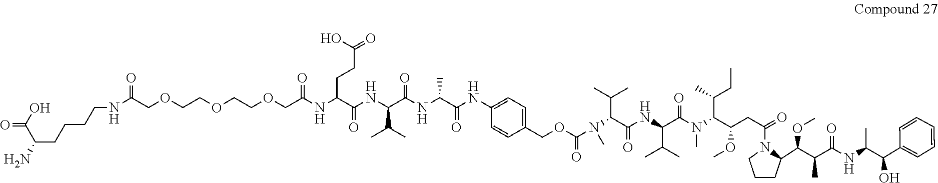 [SMILES: CC[C@@H](C)[C@H]([C@H](CC(=O)N1CCC[C@@H]1[C@@H](OC)[C@H](C)C(=O)N[C@@H](C)[C@H](O)c1ccccc1)OC)N(C)C(=O)[C@H](NC(=O)[C@@H](C(C)C)N(C)C(=O)OCc1ccc(NC(=O)[C@@H](C)NC(=O)[C@H](NC(=O)C(CCC(=O)O)NC(=O)COCCOCCOCC(=O)NCCCC[C@H](N)C(=O)O)C(C)C)cc1)C(C)C]

### Example 11: Synthesis of Compound 28

Compound 28 was synthesized by solid-phase peptide synthesis method as described in the earlier Example. The peptide 2 was conjugated with an MMAE molecule. Specifically, the MMAE molecule was linked to a β-homo-Glu[gamma-acid modified by (EG₈)₂]-Val-Cit tripeptide (peptide 2) via the connection of PABC linker, which formed an amide bond with carboxylic acid of PABC linker. The thus-synthesized molecule was then linked to a lysine residue via the connection of a linker comprising of a short PEG (CO₂H—CH₂O—CH₂CH₂O—CH₂CO₂H) and a sarcosine.

The synthesized compound 28 had the structure of Lys-PEG linker-sarcosine-3-homo-Glu(EGs)₂-Val-Cit-PABC-MMAE.

The compound 28, as illustrated below, was examined using ESI-MS and showed a molecular ion at 2371.41, corresponding to [M+H]⁺, indicating that the actual molecular weight of the compound is 2371.91 daltons.

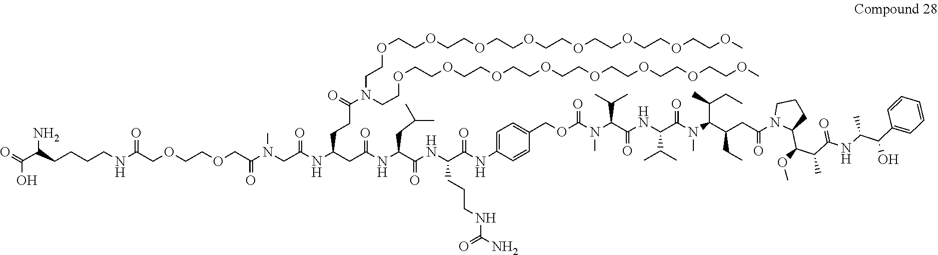

### Example 12: Synthesis of Compound 29

Peptide 11 (β-homo-Glu(EG₆)-Val-Lys(EG₈)) was synthesized by solid-phase peptide synthesis method as described in the earlier Example. The peptide 11 was conjugated with an MMAE molecule. Specifically, the MMAE molecule was linked to a β-homo-Glu[gamma-acid modified by EG₆]-Val-Lys[epsilon-amine modified by EGs]tripeptide via the connection of PABC linker, which formed an amide bond with carboxylic acid of PABC linker. The synthesized compound 29 had the structure of β-homo-Glu(EG₆)-Val-Lys(EG₈)-PABC-MMAE. The thus-synthesized molecule can be conjugated to a lysine residue via the connection of a short PEG linker (CO₂H—CH₂O—CH₂CH₂O—CH₂CO₂H).

The compound 29, as illustrated below, was examined using ESI-MS and showed a molecular ion at 955.5, corresponding to [M+2H]²⁺, indicating that the actual molecular weight of the compound is 1909 daltons.

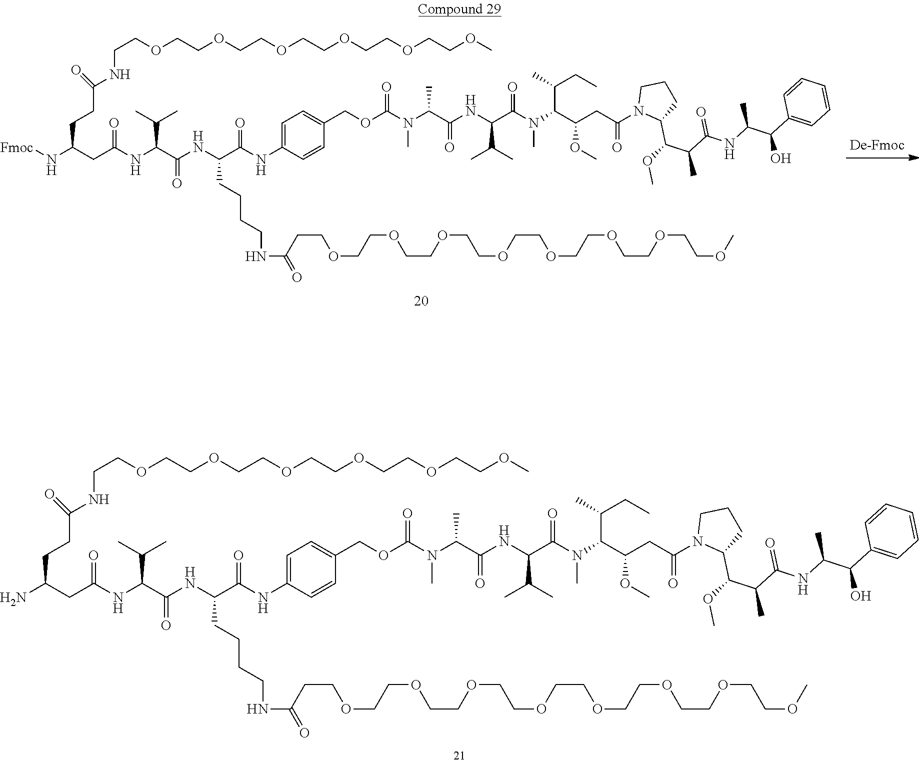

### Example 13: Synthesis of Compound 30

Compound 30 was synthesized by solid-phase peptide synthesis method as described in the earlier Example. The peptide 1 was conjugated with an acalabrutinib molecule (also known as ACP-196), which is a bruton tyrosine kinase (BTK) inhibitor. Specifically, the acalabrutinib molecule was linked to a β-homo-Glu(EG₆)-Val-Cit tripeptide (peptide 1) via the connection of PABC linker, which formed an amide bond with carboxylic acid of PABC linker. The thus-synthesized molecule can be conjugated to a lysine residue via the connection of a linker comprising of a short PEG (CO₂H—CH₂O—CH₂CH₂O—CH₂CO₂H).

The synthesized compound 30 had the structure of P3-homo-Glu(EG₆)-Val-Cit-PABC-acalabrutinib.

The compound 30, as illustrated below, was examined using ESI-MS and showed a molecular ion at 646.6, corresponding to [M+2H]²⁺, indicating that the actual molecular weight of the compound is 1291.2 daltons.

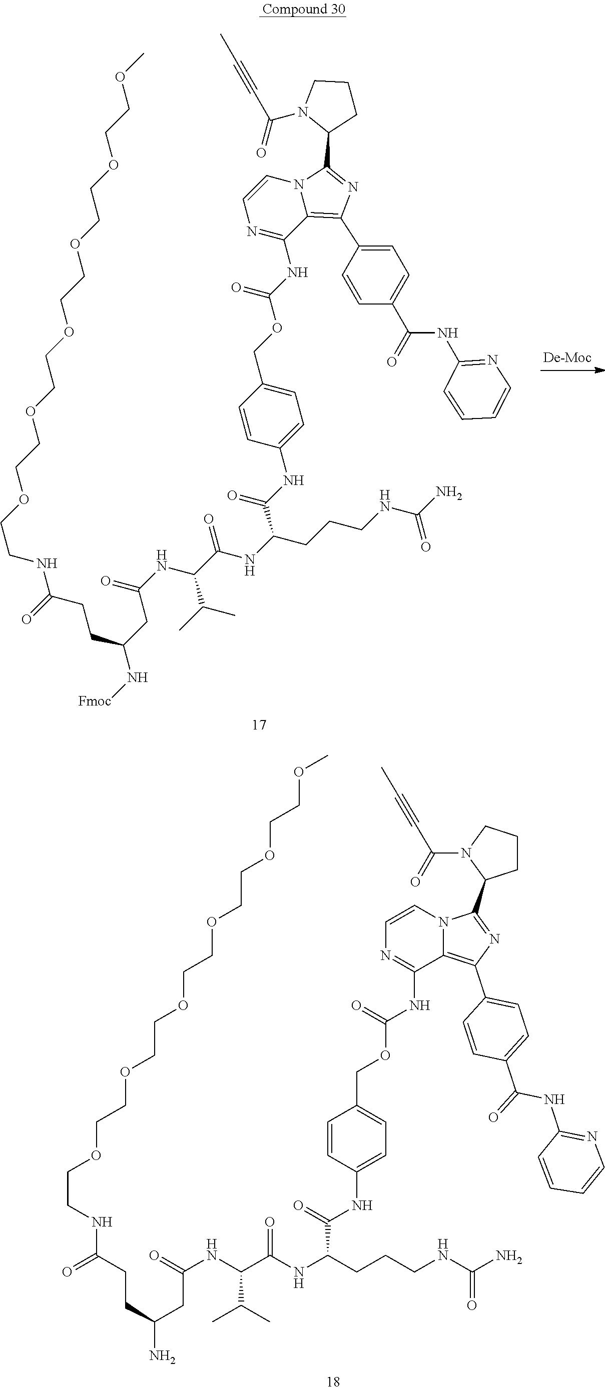

### Example 14: Synthesis of Compound 31

Compound 31 was synthesized by solid-phase peptide synthesis method as described in the earlier Example. The peptide 1 was conjugated with a niraparib molecule, which is a poly(ADP-ribose) polymerase (PARP) inhibitor (e.g., niraparib). Specifically, the niraparib molecule was linked to a β-homo-Glu(EG₆)-Val-Cit tripeptide (peptide 1) via the connection of PABC linker, which formed an amide bond with carboxylic acid of PABC linker. The thus-synthesized molecule can be conjugated to a lysine residue via the connection of a short PEG diacid linker (CO₂H—CH₂O—CH₂CH₂O—CH₂CO₂H).

The synthesized compound 31 had the structure of β-homo-Glu(EG₆)-Val-Cit-PABC-niraparib.

The compound 31, as illustrated below, was examined using ESI-MS and showed a molecular ion at 1146.9, corresponding to [M+H]⁺, indicating that the actual molecular weight of the compound is 1145.9 daltons.

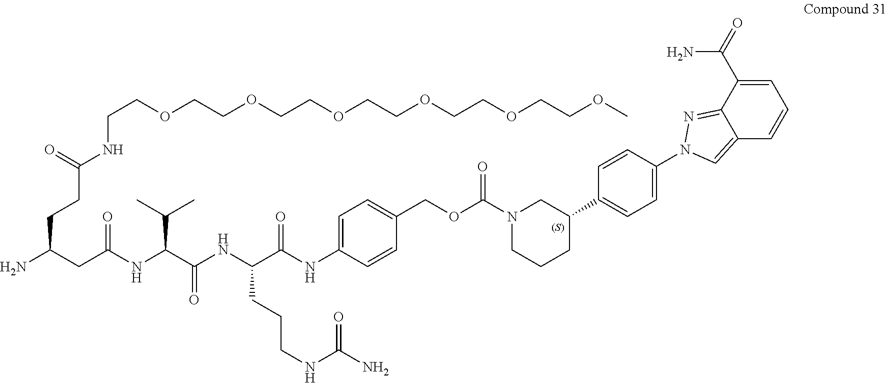

### Example 15: Synthesis of Compound 32

Peptide 12 (β-homo-Glu[(EG₁₂)₂]-Val-Cit) was synthesized by solid-phase peptide synthesis method as described in the earlier Example. The peptide 12 was conjugated with a vepdegestrant molecule (also known as ARV-471), which is a PROteolysis TArgeting Chimera (PROTAC) estrogen receptor (ER) degrader. Specifically, the vepdegestrant molecule was linked to a β-homo-Glu[(EG₁₂)₂]-Val-Cit tripeptide (peptide 12) via the connection of para-aminobenzyl alcohol (PAB)-Dimethyl Ethylenediamine (DMEDA) linker, which formed an amide bond with carboxylic acid of PAB-DMEDA linker. The thus-synthesized molecule can be conjugated to a lysine residue via the connection of a short PEG diacid linker (CO₂H—CH₂O—CH₂CH₂O—CH₂CO₂H).

The synthesized compound 32 had the structure of β-homo-Glu[(EG₁₂)₂]-Val-Cit-PAB-DMEDA-vepdegestrant.

The compound 32, as illustrated below, was examined using ESI-MS and showed a molecular ion at 1236, corresponding to [M+2H]²⁺, indicating that the actual molecular weight of the compound is 2470 daltons.

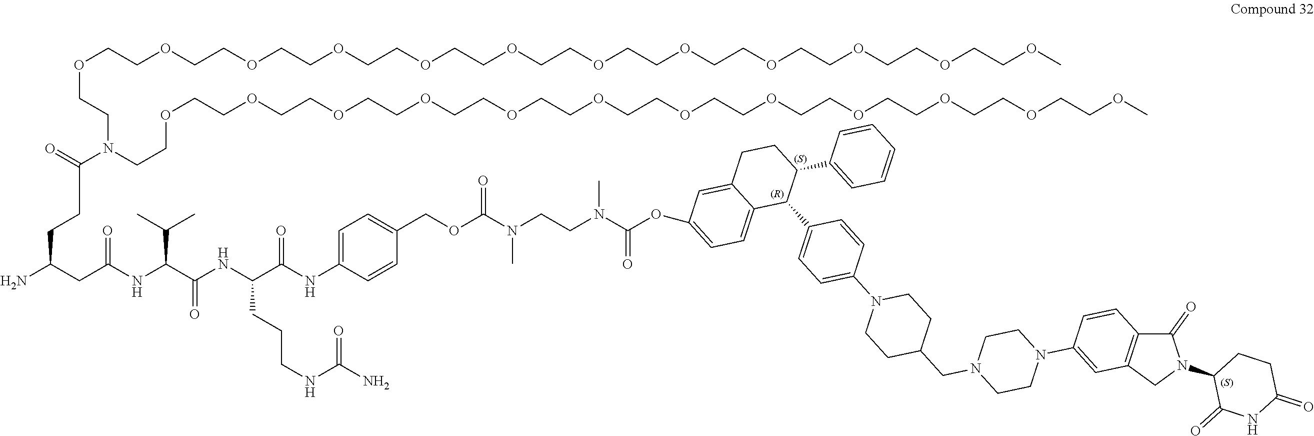

### Example 16: Synthesis of a DBCO-Containing 2-Arm Drug Bundle with Peptide 13 as a Peptide Core Conjugated with One Topoisomerase I Inhibitor (Exatecan) and One Microtubule Inhibitor (MMAF)

In this example, the DBCO-containing dual drug bundle was constructed by conjugating peptide 13 with one exatecan molecule and one MMAF molecule. The peptide 13 has the structure of DBCO GSGKGSGKG (SEQ ID NO: 1). Similar to previous Example, in the dual bundle, the exatecan molecule and MMAF molecule were individually linked to lysine residues of compound 5 flanked through different linking arms. Specifically, one exatecan molecule was linked to an β-homo-Glu[gamma-acid modified by EG₆]-Val-Cit tripeptide (peptide 1) via the connection of PABC linker, which formed an amide bond with carboxylic acid of PABC linker. The thus-synthesized molecule was then linked to lysine residue of peptide 13 via the connection of a short PEG diacid linker. On the other hand, the MMAF molecule was directly linked to lysine residue of peptide 13 via the connection of a short PEG diacid linker.

The DBCO-containing dual drug bundle having one exatecan molecule and one MMAF molecule was synthesized using the combined method of standard Fmoc-based solid-phase synthesis for central core and liquid-phase synthesis for linking DBCO-containing group, the exatecan-containing linking arm, and the MMAF-containing linking arm to the side chain of lysine residue of peptide core via amide bond formation.

As illustrated below, the DBCO-containing drug bundle comprised a peptide core derived from peptide 13, with one linking arm (β-homo-Glu(EG₆)-Val-Cit) (peptide 1) carrying an exatecan molecule and another linking arm (i.e., (EG₂) carrying an MMAF molecule. The identification of the synthesized dual drug bundle having one exatecan molecule and one MMAF molecule was carried out by mass spectrometry ESI-MS. The present molecular construct showed a molecular ion at 1650.32, corresponding to [M+2H]²⁺, indicating that the actual molecular weight of the drug bundle is 3298.64 daltons. The reversed-phase HPLC profile of the present DBCO-containing drug bundle (compound 33) showed the peak of the synthesized drug bundle appearing at a retention time of 25.957 min.

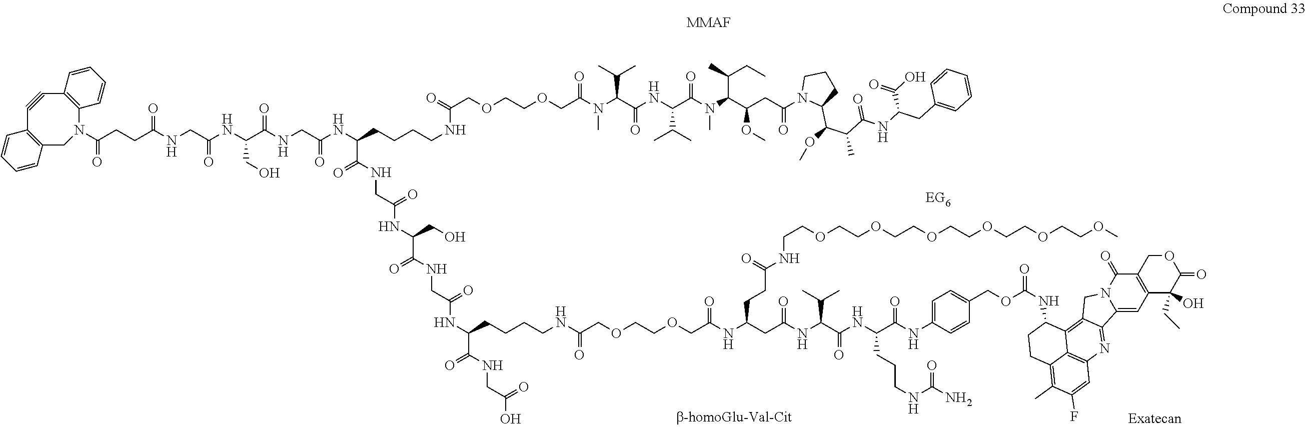

As illustrated below, the DBCO-containing drug bundle comprised a peptide core derived from peptide 13, with one linking arm (β-homo-Glu[(EG₈)₂]-Val-Cit) (peptide 2) carrying an exatecan molecule and another linking arm (EG₂) carrying an MMAF molecule. The identification of the synthesized dual drug bundle having one exatecan molecule and one MMAF molecule was carried out by mass spectrometry ESI-MS. The present molecular construct showed a molecular ion at 1877.44, corresponding to [M+2H]²⁺, indicating that the actual molecular weight of the drug bundle is 3752.88 daltons. The reversed-phase HPLC profile of the present DBCO-containing drug bundle (compound 34) showed the peak of the present drug bundle appearing at a retention time of 26.297 min.

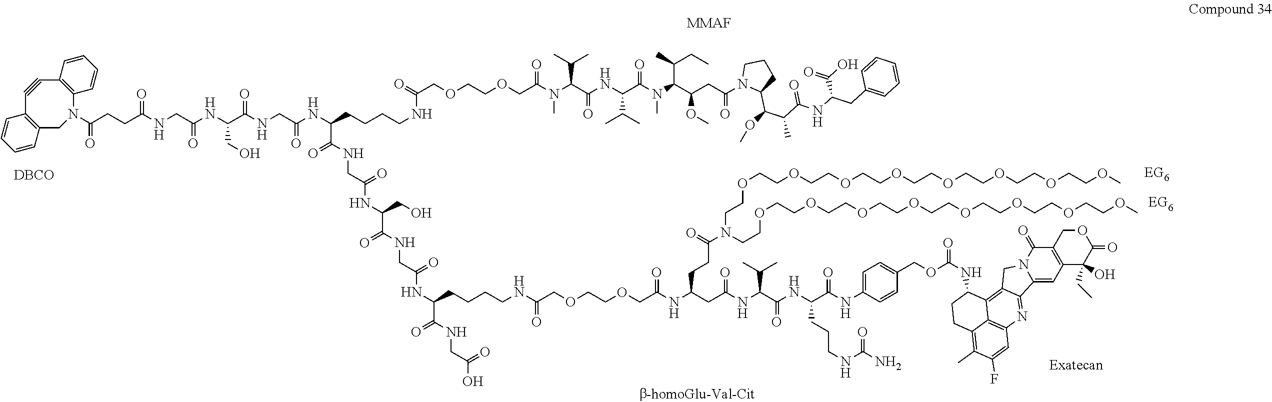

### Example 17: Synthesis of a DBCO-Containing 2-Arm Drug Bundle with Peptide 13 Conjugated with One Topoisomerase I Inhibitor (Exatecan) and One Microtubule Inhibitor (MMAE)

In this example, the DBCO-containing dual drug bundle was synthesized by using peptide 13 as a peptide core, which was conjugated with one exatecan molecule and one MMAE molecule. In the dual drug bundle, the exatecan and MMAE molecules were individually linked to lysine residues of peptide 13 flanked through different linking arms. Specifically, the exatecan molecule was linked to an Glu[gamma-acid modified by EG₆]-Val-Cit tripeptide (peptide 1) via the connection of PABC linker, which formed an amide bond with carboxylic acid of PABC linker. The thus-synthesized molecule was then linked to lysine residue of peptide 13 via the connection of a short PEG diacid linker. Similarly, the MMAE molecule was linked to a Glu-[gamma-acid modified by (EG₆)]-Val-Cit tripeptide via the connection of PABC linker, which formed an amide bond with carboxylic acid of PABC linker. The thus-synthesized molecule was then linked to lysine residue of peptide 13 via the connection of a short PEG diacid linker.

As illustrated below, the DBCO-containing drug bundle comprises a peptide core derived from peptide 13, with one linking arm (β-homo-Glu(EG₆)-Val-Cit) (peptide 1) carrying an exatecan molecule and another linking arm (β-homo-Glu(EG₆)-Val-Cit) (peptide 1) carrying an MMAE molecule. The identification of the synthesized dual drug bundle having one exatecan molecule and one MMAE molecule was carried out by mass spectrometry ESI-MS. The present molecular construct showed a molecular ion at 1371.0311, corresponding to [M+3H]³⁺, indicating that the actual molecular weight of the drug bundle is 4110.0933 daltons (deconvolution). The reversed-phase HPLC profile of the present DBCO-containing drug bundle (compound 35) showed the peak of the present drug bundle appearing at a retention time of 26.248 min.

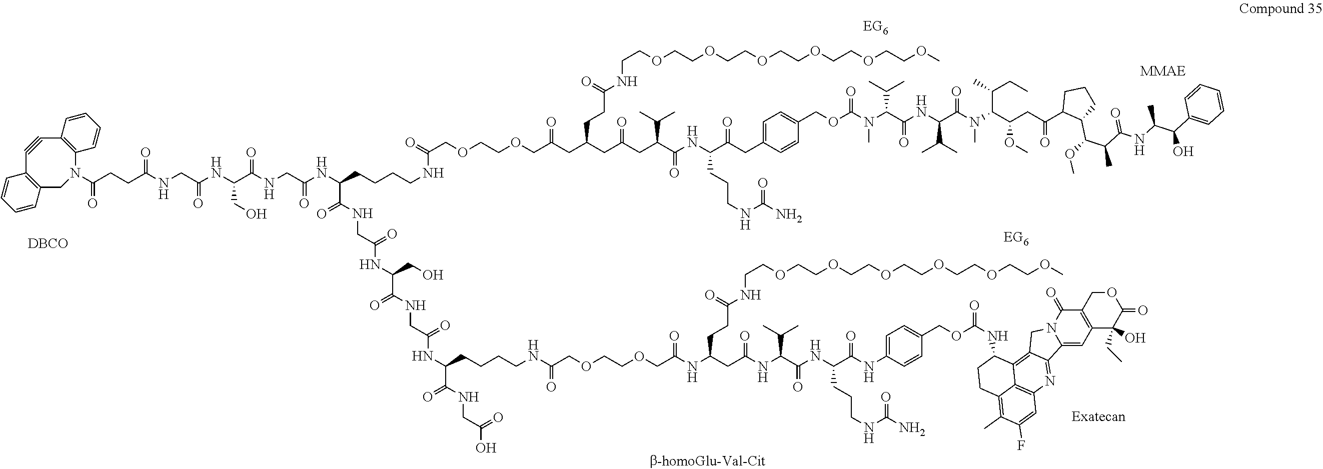

As illustrated below, the DBCO-containing drug bundle comprised a peptide core derived from peptide 13, with one linking arm (β-homo-Glu(EG₆)-Val-Lys(EGs)) (peptide 11) carrying an MMAE molecule and another linking arm (β-homo-Glu(EG₆)-Val-Cit) (peptide 1) carrying an exatecan molecule. The identification of the synthesized dual drug bundle having one MMAE molecule and one exatecan molecule was carried out by mass spectrometry ESI-MS. The present molecular construct showed a molecular ion at 1492.8371, corresponding to [M+3H]³⁺, indicating that the actual molecular weight of the drug bundle is 4475.5113 daltons. The reversed-phase HPLC profile of the present DBCO-containing drug bundle (compound 36), with the peak of the present drug bundle appearing at a retention time of 10.007 min.

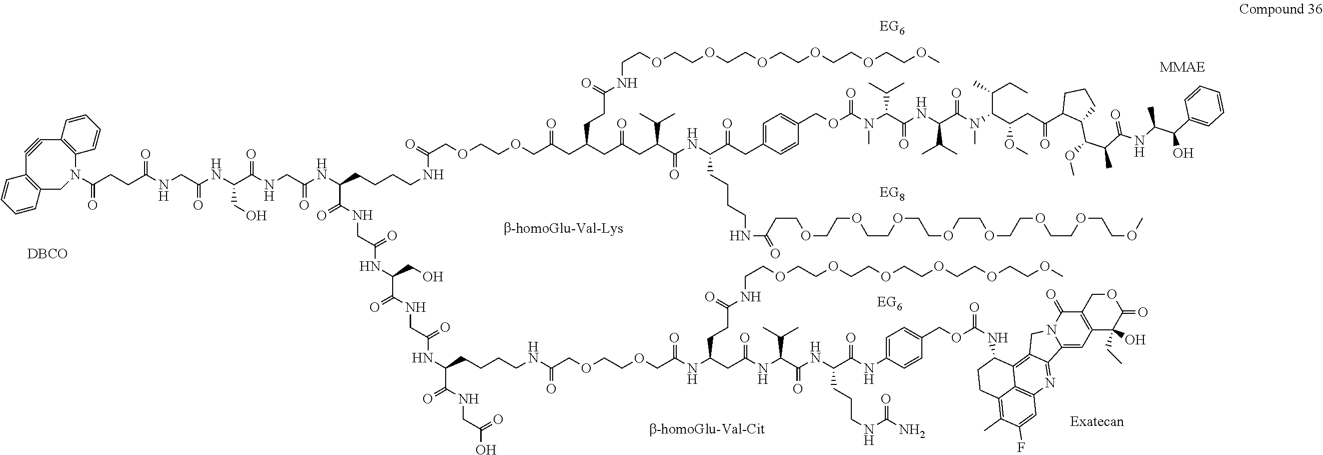

### Example 18: Synthesis of a DBCO-Containing 2-Arm Drug Bundle with Peptide 13 as a Peptide Core Conjugated with One Microtubule Inhibitor (MMAF) and One Bruton Tyrosine Kinase (BTK) Inhibitor (Acalabrutinib)

In this example, the DBCO-containing dual drug bundle was synthesized using peptide 14 as the peptide core, which was conjugated with one microtubule inhibitor (MMAF) molecule and one bruton tyrosine kinase (BTK) inhibitor (acalabrutinib) molecule. In the dual drug bundle, the MMAF and acalabrutinib molecules were individually linked to lysine residues of peptide 13 flanked through different linking arms. Specifically, the MMAF molecule was directly linked to lysine residue of peptide 13 via the connection of a short PEG diacid linker. On the other hand, the acalabrutinib molecule was linked to an Glu[gamma-acid modified by EG₆]-Val-Cit tripeptide (peptide 1) via the connection of PABC linker, which formed an amide bond with carboxylic acid of PABC linker. The thus-synthesized molecule was then linked to lysine residue of peptide 13 via the connection of a short PEG diacid linker.

As illustrated below, the DBCO-containing drug bundle comprises a peptide core derived from peptide 13, with one linking arm (EG₂) carrying an MMAF molecule and another linking arm (β-homo-Glu(EG₆)-Val-Cit) (peptide 1) carrying an acalabrutinib molecule. The identification of the synthesized dual drug bundle having one MMAF molecule and one acalabrutinib molecule was carried out by mass spectrometry ESI-MS. The present molecular construct showed a molecular ion at 1110.6, corresponding to [M+3H]³⁺, indicating that the actual molecular weight of the drug bundle is 3328.8 daltons (deconvolution). The reversed-phase HPLC profile of the present DBCO-containing drug bundle (compound 37) showed the peak of the present drug bundle appearing at a retention time of 11.198 min.

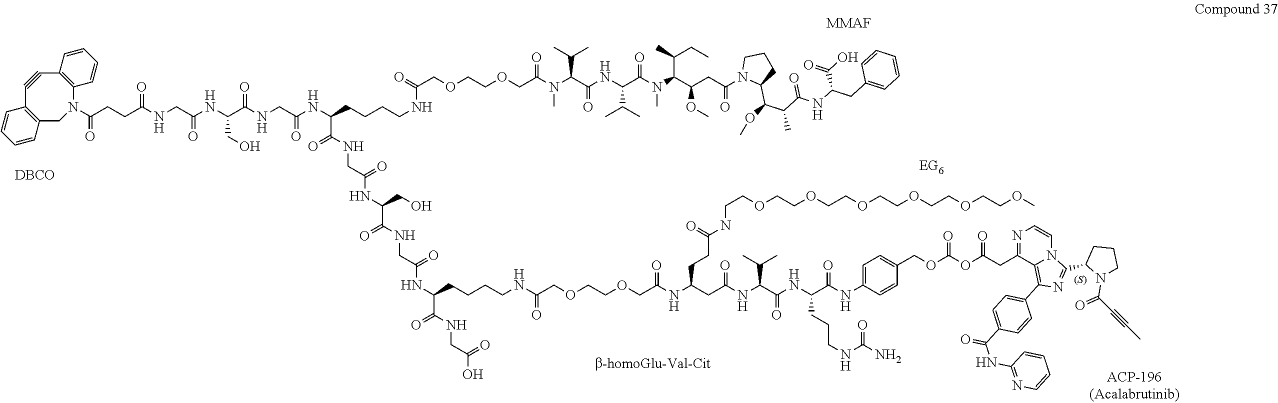

### Example 19: Synthesis of a DBCO-Containing 2-Arm Drug Bundle with Peptide 13 as a Peptide Core Conjugated with One Topoisomerase I Inhibitor (Exatecan) and One Poly(ADP-Ribose) Polymerase (PARP) Inhibitor (Niraparib)

In this example, the DBCO-containing dual drug bundle, using peptide 13 as a peptide core, was conjugated with one exatecan molecule and one poly(ADP-ribose) polymerase (PARP) inhibitor (i.e., niraparib) molecule. In the dual drug bundle, the exatecan and niraparib molecules were individually linked to lysine residues of peptide 13 flanked through different linking arms. Specifically, the exatecan molecule was linked to an Glu[gamma-acid modified by EG₆]-Val-Cit tripeptide via the connection of PABC linker, which formed an amide bond with carboxylic acid of PABC linker. The thus-synthesized molecule was then linked to lysine residue of peptide 13 via the connection of a short PEG diacid linker. Similarly, the niraparib molecule was linked to a Glu[gamma-acid modified by EG₆]-Val-Cit tripeptide (peptide 1) via the connection of PABC linker, which formed an amide bond with carboxylic acid of PABC linker. The thus-synthesized molecule was then linked to lysine residue of peptide 13 via the connection of a short PEG diacid linker.

As illustrated below, the DBCO-containing drug bundle comprises a peptide core derived from peptide 13, with one linking arm (β-homo-Glu(EG₆)-Val-Cit) (peptide 1) carrying an exatecan molecule and another linking arm (β-homo-Glu(EG₆)-Val-Cit) (peptide 1) carrying a niraparib molecule. The identification of the synthesized dual drug bundle having one exatecan molecule and one niraparib molecule was carried out by mass spectrometry ESI-MS. According to the data of ESI-MS, the present molecular construct showed a molecular ion at 1238.58, corresponding to [M+3H]³⁺, indicating that the actual molecular weight of the drug bundle is 3712.74 daltons (deconvolution). The reversed-phase HPLC profile of the present DBCO-containing drug bundle (compound 38) showed the peak of the present drug bundle appearing at a retention time of 9.654 min.

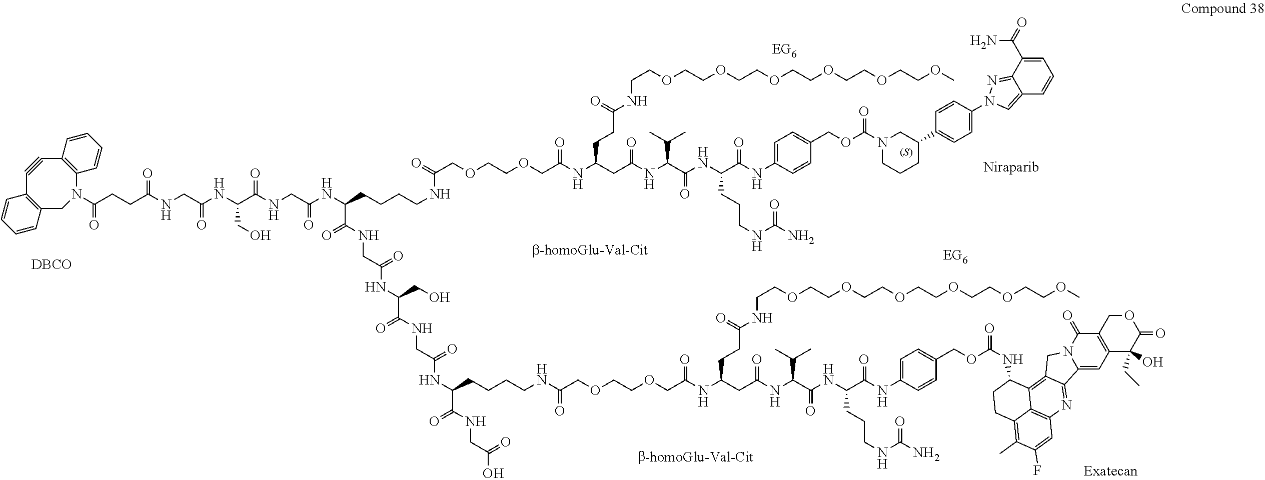

### Example 20: Synthesis of a DBCO-Containing 2-Arm Drug Bundle with Peptide 13 as a Peptide Core Conjugated with One Topoisomerase I Inhibitor (Exatecan) and One PROTAC of Degrading Estrogen Receptors (Vepdegestrant)

In this example, the DBCO-containing dual drug bundle, using peptide 13 as the peptide core, was conjugated with one exatecan molecule and one PROTAC of degrading estrogen receptors (i.e., vepdegestrant (ARV-471). In the dual drug bundle, the exatecan and vepdegestrant molecules were individually linked to lysine residues of peptide 13 flanked through different linking arms. Specifically, the exatecan molecule was linked to an Glu[gamma-acid modified by EG₆]-Val-Cit tripeptide (peptide 1) via the connection of PABC linker, which formed an amide bond with carboxylic acid of PABC linker. The thus-synthesized molecule was then linked to lysine residue of peptide 13 via the connection of a short PEG diacid linker. Similarly, the vepdegestrant molecule was linked to a Glu[gamma-acid modified by branched EG linker (EG₁₂)₂]-Val-Cit tripeptide (peptide 12) via the connection of PAB-DMEDA linker, which formed an amide bond with carboxylic acid of PAB-DMEDA linker. The thus-synthesized molecule was then linked to lysine residue of peptide 13 via the connection of a short PEG diacid linker.

As illustrated below, the DBCO-containing drug bundle comprises a peptide core derived from peptide 13, with one linking arm (β-homo-Glu(EG₆)-Val-Cit) (peptide 1) carrying an exatecan molecule and another linking arm (β-homo-Glu[(EG₁₂)₂]-Val-Cit) (peptide 12) carrying a vepdegestrant molecule. The identification of the synthesized dual drug bundle (compound 39) having one exatecan molecule and one vepdegestrant molecule was carried out by mass spectrometry ESI-MS.

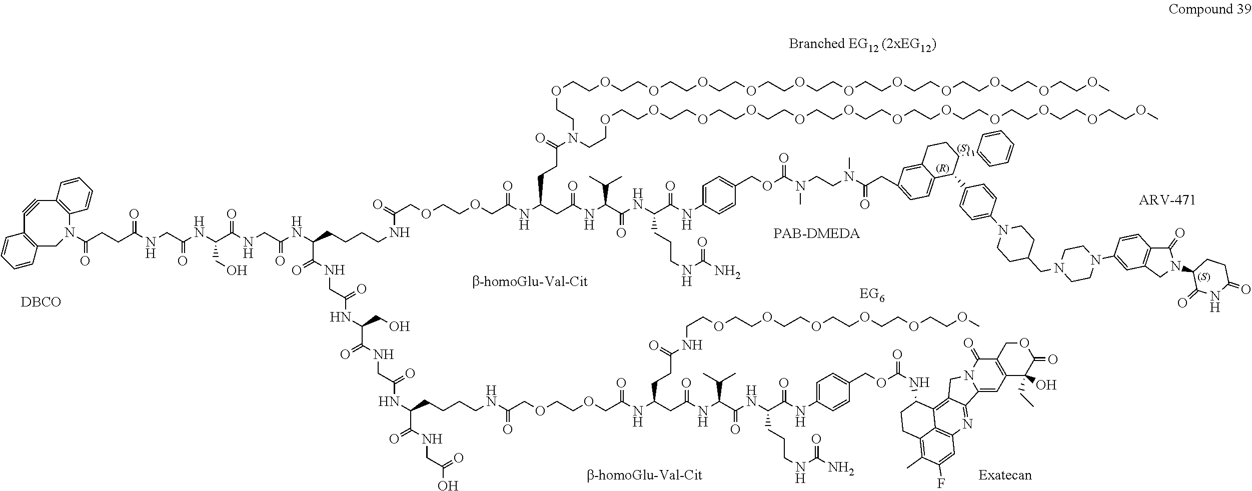

### Example 21: Synthesis of a BCN-Containing 2-Arm Drug Bundle (i.e., Two Exatecan Molecules) with Peptide 14 as the Peptide Core

In this example, the BCN-containing drug bundle, using peptide 14 (BCN-GSGKGSGKG; SEQ ID NO:1) as a peptide core, was conjugated with two exatecan molecules.

As illustrated below, the BCN-containing drug bundle comprised a peptide core derived from peptide 14 and two linking arms (β-homo-Glu(EG₆)-Val-Cit) (peptide 1), each capable of carrying an exatecan molecule, for a total of two payloads. The identification of the synthesized topoisomerase I inhibitor bundle was carried out by mass spectrometry ESI-MS. According to the data, the present molecular construct showed a molecular ion at 1240.1600 corresponding to [M+3H]³⁺, indicating that the actual molecular weight of the drug bundle is 3717.48 daltons (deconvolution). The reversed-phase HPLC profile of the present BCN-containing drug bundle (compound 40) showed the peak of the present drug bundle appearing at a retention time of 25.082

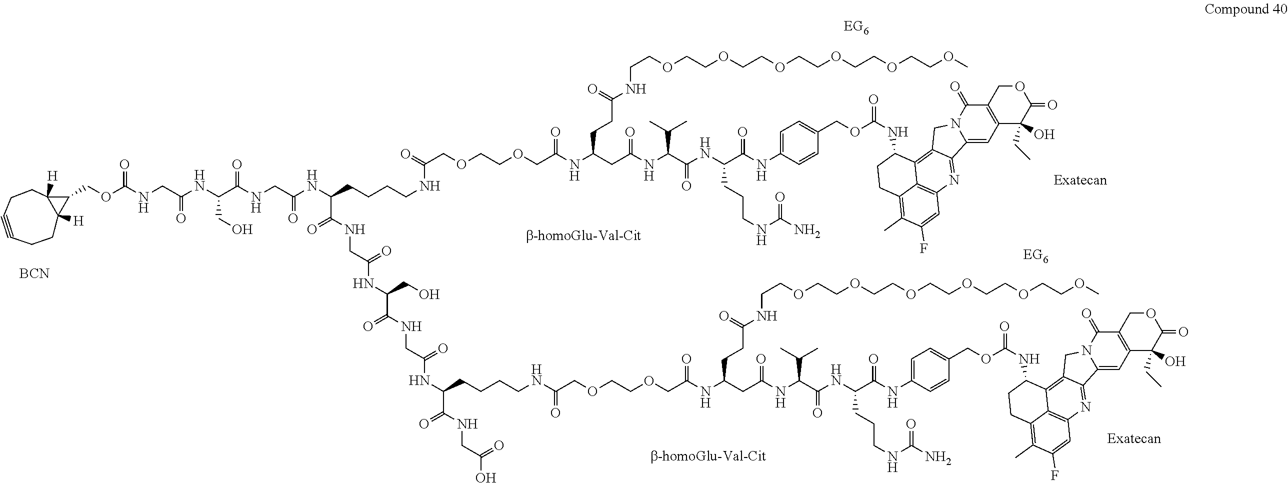

### Example 22: Synthesis of a BCN-Containing 2-Arm Drug Bundle with Peptide 15 as a Peptide Core Conjugated with Two Topoisomerase I Inhibitors (Exatecan)

In this example, the BCN-containing drug bundle, using peptide 15 (BCN-EG₂-GSGKGSGKG; SEQ ID NO: 1) as a peptide core, was conjugated with two exatecan molecules.

As illustrated below, the BCN-containing drug bundle comprised a peptide core of derived from peptide 15 and two linking arms (β-homo-Glu(EG₆)-Val-Cit) (peptide 1), each capable of carrying an exatecan molecule, for a total of two payloads. The identification of the synthesized topoisomerase I inhibitor exatecan bundle was carried out by mass spectrometry ESI-MS. According to the data, the present molecular construct showed a molecular ion at 1288.5600 corresponding to [M+3H]³⁺, indicating that the actual molecular weight of the drug bundle is 3862.68 daltons. The reversed-phase HPLC profile of the present BCN-containing drug bundle (compound 41) showed the peak of the present drug bundle appearing at a retention time of 25.253 min.

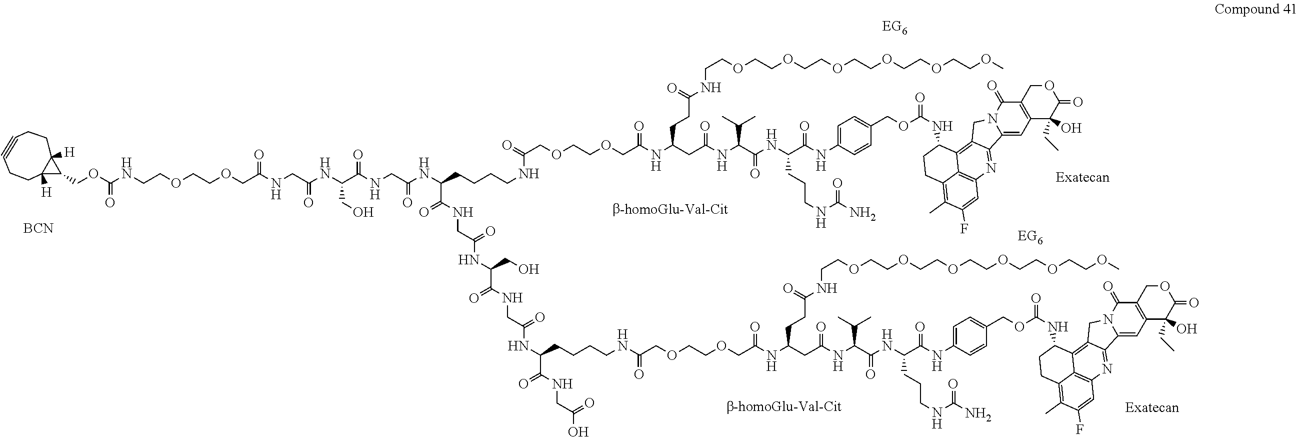

### Example 23: Synthesis of a DBCO-Containing 3-Arm Drug Bundle with Peptide 16 as a Peptide Core Conjugated with One Topoisomerase I Inhibitor (Exatecan) Plus Two Microtubule Inhibitors (MMAE and MMAF)

In this example, the DBCO-containing drug bundle, using peptide 16 (DBCO-GSGKGSGKGSGKG; SEQ ID NO: 2) as a peptide core, was conjugated with dual drug, including one topoisomerase I inhibitor (exatecan) and two microtubule inhibitors (MMAE and MMAF).

As illustrated below, the DBCO-containing drug bundle comprised a peptide core derived from peptide 16, with one linking arm (β-homo-Glu(EG₆)-Val-Cit) (peptide 1) carrying an exatecan molecule, one linking arm (β-homo-Glu(EG₆)-Val-Cit) (peptide 1) carrying an MMAE molecule, and one linking arm (EG₂) carrying an MMAF molecule. The identification of the synthesized dual drug bundle was carried out by mass spectrometry ESI-MS. The present molecular construct showed a molecular ion at 1329.6400, corresponding to [M+4H]⁴⁺, indicating that the actual molecular weight of the drug bundle is 5314.56 daltons. The reversed-phase HPLC profile of the present DBCO-containing drug bundle (compound 42) showed the peak of the present drug bundle appearing at a retention time of 26.704 min.

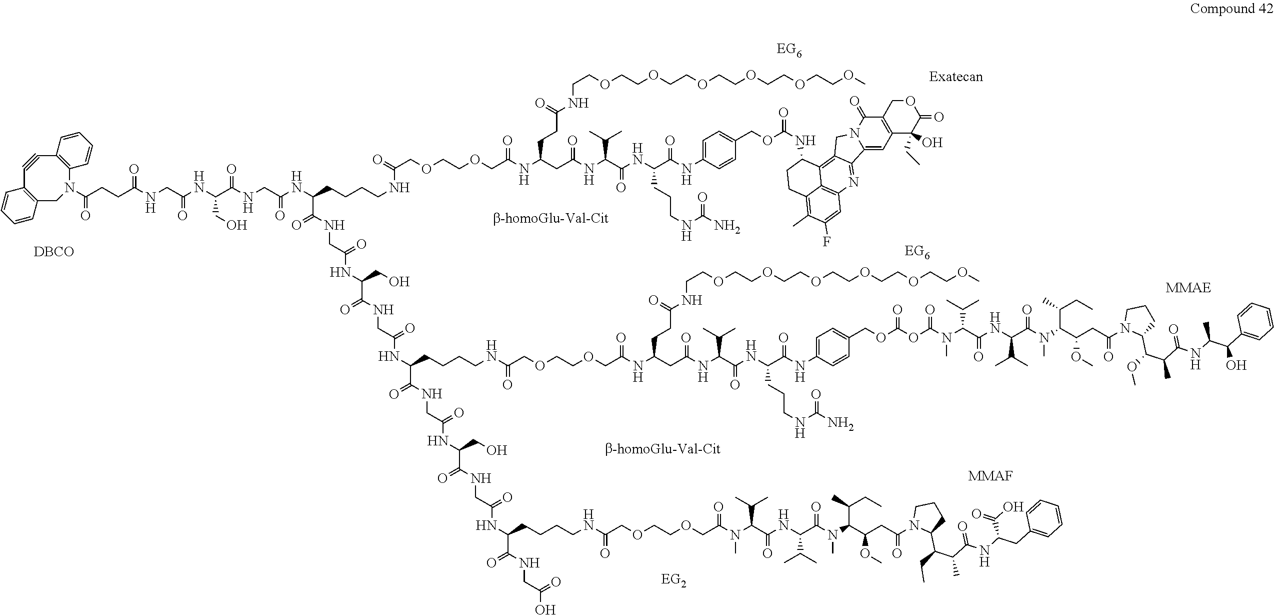

### Example 24: Synthesis of a BCN-Containing 4-Arm Drug Bundle with Peptide 17 as a Peptide Core Conjugated with Three Topoisomerase I Inhibitors (Exatecan) and One Microtubule Inhibitor (MMAE)

In this example, the BCN-containing drug bundle, using peptide 17 (BCN-EG₂-GSGKGSGKGSGKGSGKG; SEQ ID NO: 3) as a peptide core, was conjugated with dual drug, including three topoisomerase I inhibitors (exatecan) and one microtubule inhibitor (MMAE).

As illustrated below, the BCN-containing drug bundle comprised a peptide core derived from peptide 17, three linking arms (β-homo-Glu(EG₆)-Val-Cit) (peptide 1) each carrying an exatecan molecule, and one additional linking arm (β-homo-Glu(EG₆)-Val-Cit) (peptide 1) for carrying an MMAE molecule. The identification of the synthesized dual drug bundle was carried out by mass spectrometry ESI-MS. The present molecular construct showed a molecular ion at 1903.4433, corresponding to [M+4H]⁴⁺, indicating that the actual molecular weight of the drug bundle is 7609.7732 daltons. The reversed-phase HPLC profile of the present DBCO-containing drug bundle (compound 43) showed the peak of the present drug bundle appearing at a retention time of 27.112 min.

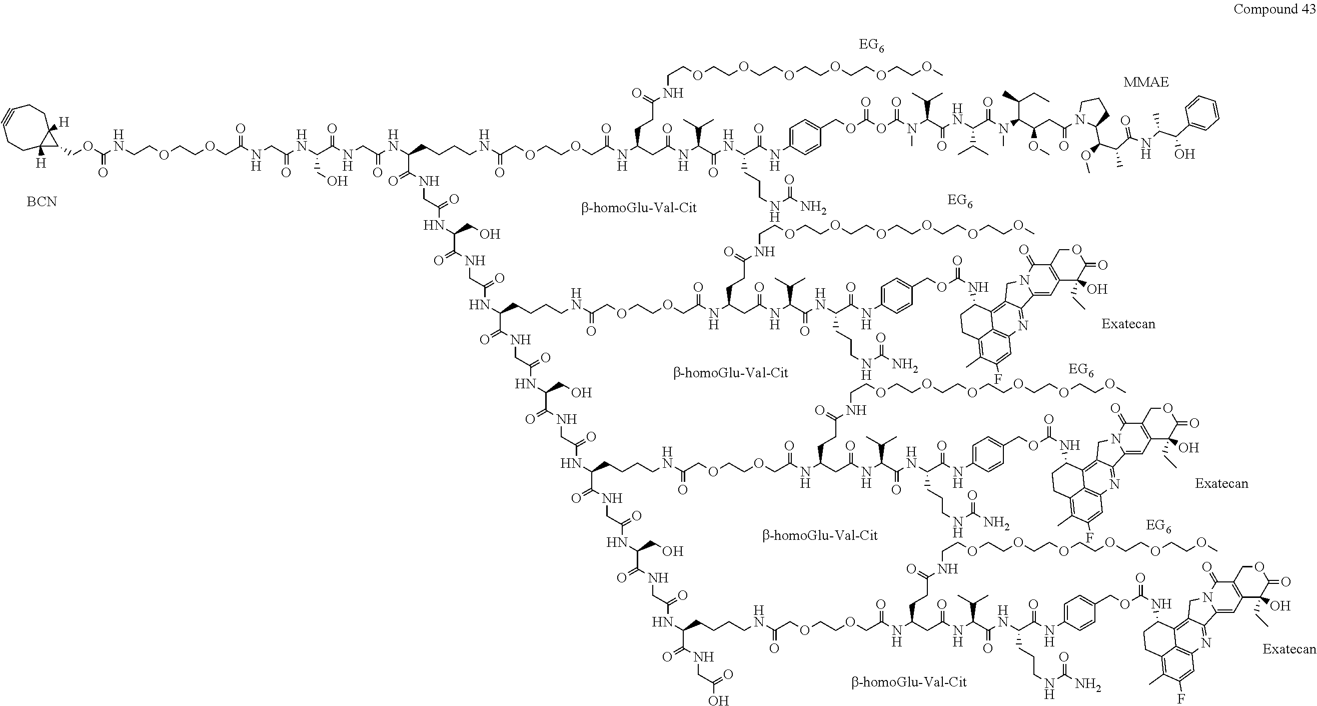

### Example 25: Synthesis of a DBCO-Containing Peptide with One Linking Arm Conjugated with One Topoisomerase I Inhibitors (Exatecan)

In this example, the DBCO-containing linking arm was conjugated with one topoisomerase I inhibitors (exatecan).

As illustrated below, the DBCO-containing linking arm with one payload comprised a DBCO group, a tetrapeptide (Gly-Ser-Gly-Lys), and a linking arm (β-homo-Glu(EG₆)-Val-Cit) (peptide 1) carrying an exatecan molecule. The identification of the synthesized compound was carried out by mass spectrometry ESI-MS. The present molecular construct showed a molecular ion at 2037.89, corresponding to [M+H]⁺, indicating that the actual molecular weight of the drug bundle is 2036.89 daltons. The reversed-phase HPLC profile of the present DBCO-containing linking arm with one payload (compound 44) showed the peak of the present drug bundle appearing at a retention time of 10.255 min.

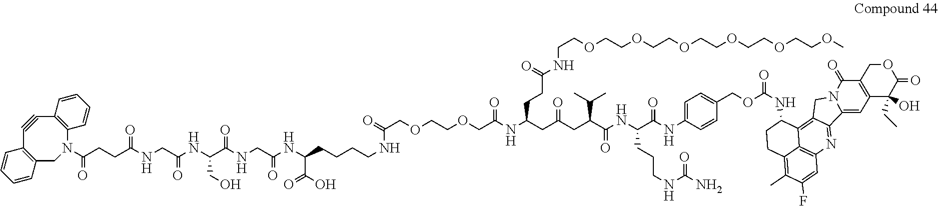 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](CCCNC(N)=O)NC(=O)[C@@H](CC(=O)C[C@H](CCC(=O)NCCOCCOCCOCCOCCOCCOC)NC(=O)COCCOCC(=O)NCCCC[C@H](NC(=O)CNC(=O)[C@H](CO)NC(=O)CNC(=O)CCC(=O)N2Cc4ccccc4C#Cc4ccccc42)C(=O)O)C(C)C)cc1)CC3]

### Example 25: Analysis of Enzymatic Cleavage of the Linking Arm of a Drug Bundle Carrying One Exatecan Molecule by Recombinant Human Neutrophile Elastase (HNE)

It is known that HNE, an enzyme secreted extracellularly from differentiating human neutrophils, promotes the release of cytotoxic drugs from Val-Cit-based ADCs during circulation, potentially leading to a reduction in bone marrow neutrophil populations and other off-target toxicities.

To identify more stable linking arms carrying the exatecan molecule, beyond the Val-Cit dipeptide or Glu-Val-Cit tripeptide, synthesized peptides were tested and screened for improved stability and resistance against HNE-mediated degradation. To assess the cleavability of the linking arms by neutrophil elastase, an HNE-mediated cleavage assay was conducted. Briefly, neutrophil elastase was incubated with 20 μg of the building blocks in 100 mM Tris and 500 mM NaCl at pH 7.5, and digested with 2 nM HNE at 25° C. for 48 hours. The elastase-digested samples were frozen until analysis by C18 RP-HPLC.

The cleaved exatecan cytotoxic drug was analyzed using a C18 RP-HPLC column (4.6×250 mm, 5 μm). The initial conditions were set at 100% solvent A (0.1% TFA in water) and 0% solvent B (ACN with 0.1% TFA), with solvent B being linearly increased to 100% over 20 minutes. The flow rate was maintained at 1 mL/min, the column temperature was set at 25° C., the detection wavelength was 220 nm, and the injection volume was 20 μL.

According to results provided in Table 1, the free drug release rate of the linking arm carrying one MMAE molecule with Val-Cit within 48 hours by HNE was 5.6%. In contrast, the linking arms carrying one MMAE molecule with β-homo-Glu-Val-Cit (peptide 3), β-homo-Glu(EG₆)-Val-Cit (peptide 1), and β-homo-Glu[(EG₈)₂]-Val-Cit (peptide 2) tripeptides exhibited high resistance to HNE. The free drug release rates for these linking arms within 48 hours by HNE were 0.4%, 1.16%, 1.36%, and 0%, respectively. These results suggested that β-homo-Glu at the P3 position in the P3-P2-P1 tripeptide structure of the linking arms carrying one exatecan molecule confers significant resistance to HNE cleavage.

TABLE 1

| | | % of drug |
|---|---|---|
| | | released after |
| | | HNE digestion |
| Name | Structure | for 48 hr |
| Compound 17 | Lys-PEG linker-β-homo-Glu(EG₆)-Val- | 1.36% |
| | Cit-PABC-EXT | |
| Compound 18 | Lys-PEG linker-β-homo-Glu[(EG₈)₂]- | 0% |
| | Val-Cit-PABC-EXT | |
| Compound 19 | Lys-PEG linker-β-homo-Glu-Val-Cit- | 0.4% |
| | PABC-EXT | |
| Compound 21 | Lys-PEG linker-Val-Cit-PABC-EXT | 5.56% |

Moreover, the insertion of β-homo-Glu (with or without PEGylation) or methylGlu residues at the P3 position of the P3-P2-P1 tripeptide (e.g., β-homo-Glu-Val-Cit) drastically reduced cleavage by HNE, strongly suggesting that the additional —CH₂— group in the backbone of β-homo-Glu at the P3 position made it a less suitable substrate for HNE.

### Example 26: Analysis of Enzymatic Cleavage of the Linking Arm of a Drug Bundle Carrying One MMAE Molecule by Recombinant HNE

The HNE-mediated cleavage assay was conducted as described in a previous example. The results of the MMAE release assay for the linking arm carrying one MMAE molecule were provided in Table 2. According to the results of Table 2, the linking arm carrying one MMAE molecule with the Glu-Val-Ala tripeptide is highly susceptible to cleavage by HNE, with a free drug release rate of up to 83% within 48 hours; and the linking arm carrying one MMAE molecule with the Glu-Val-Cit tripeptide demonstrated slightly greater resistance to HNE, with a free drug release rate of 41.3% within 48 hours.

For linking arms carrying one MMAE molecule with Glu(EG₃)-Val-Cit (peptide 7) and Glu(EG₆)-Val-Cit (peptide 1) tripeptides, the PEGylation of the P3 residue appeared to have a limited impact on resistance to HNE cleavage. Both PEGylated Glu-Val-Cit tripeptides were susceptible to cleavage, with free drug release rates of 75% and 71.8%, respectively, which were even higher than those of the Glu-Val-Ala tripeptide.

In contrast, the linking arms of β-homo-Glu-Val-Cit and methylGlu-Val-Cit tripeptides carrying one MMAE molecule exhibited high resistance to HNE, with no detectable free drug release within 48 hours. Notably, the linking arm of the sarcosine-containing β-homo-Glu-Val-Cit short peptide carrying one MMAE molecule also exhibited the same high resistance to HNE.

TABLE 2

| | | % of drug |
|---|---|---|
| | | released after |
| | | HNE digestion |
| Name | Structure | for 48 hr |
| Compound 23 | Lys-PEG linker-Glu(EG₃)-Val-Cit- | 75% |
| | PABC-MMAE | |
| Compound 24 | Lys-PEG linker-β-homo-Glu-Val-Cit- | 0.0% |
| | PABC-MMAE | |
| Compound 25 | Lys-PEG linker-methylGlu-Val-Cit- | 0.0% |
| | PABC-MMAE | |
| Compound 26 | Lys-PEG linker-Glu-Val-Cit-PABC- | 41.3% |
| | MMAE | |
| Compound 27 | Lys-PEG linker-Glu-Val-Ala-PABC- | 83.0% |
| | MMAE | |
| Compound 28 | Lys-PEG linker-sacrosine-β-homo- | 0.0% |
| | Glu[(EG₈)₂]-Leu-Cit-PABC-MMAE | |

These findings suggest that, compared to Glu, β-homo-Glu at the P3 position in the P3-P2-P1 tripeptide structure confers greater resistance to HNE cleavage. Moreover, replacing Glu with β-homo-Glu significantly reduced cleavage by HNE, strongly suggesting that the additional —CH₂— group in the backbone of β-homo-Glu at the P3 position renders it a less suitable substrate for HNE.

### Example 27: Drug Release from the Linking Arm of a Drug Bundle Carrying One Exatecan Molecule Via Recombinant Cathepsin B-Mediated Cleavage

Cathepsin B (CatB) is an intracellular enzyme localized in the lysosomes of normal human cells and plays a crucial role in intracellular proteolysis. CatB-recognized cleavable linkers, such as the Val-Cit linker, are commonly used in ADCs to release free cytotoxic drugs within target cells. Therefore, CatB has been widely utilized in in vitro drug release assays. In these assays, CatB is added to media or solutions containing ADCs, which induces the release of free cytotoxic drugs from cleavable linkers. The amount of released free drug is then quantified. Specifically, in these studies, after CatB is added to ADC solutions and incubated at 37° C., the amount of released MMAE cytotoxic drug is quantified by HPLC. This CatB-mediated drug release assay is essential for comparing and selecting linkers that can efficiently release free drugs from ADCs intracellularly.

To examine the cleavability of linking arms by recombinant CatB, 20 μg of the building blocks were incubated with cathepsin B at room temperature in 50 mM sodium citrate phosphate (pH 5.0) and digested with 0.27 μM CatB enzyme at 37° C. for 48 hours. The CatB-digested samples were then frozen until analysis by C18-RP HPLC.

The cleaved exatecan cytotoxic drug was analyzed using a C18 RP-HPLC column (4.6×250 mm, 5 μm). The initial conditions were set at 100% solvent A (0.1% TFA in water) and 0% solvent B (ACN with 0.1% TFA). Solvent B was linearly increased to 100% over 20 minutes. The flow rate was maintained at 1 mL/min, the column temperature was set at 25° C., the detection wavelength was 220 nm, and the injection volume was 20 μL.

The results of the exatecan release assay from the linking arm carrying one exatecan molecule, as cleaved by CatB, were summarized in Table 3. The data in Table 3 demonstrated that the exatecan release rates from linking arms carrying one exatecan molecule with 3-homo-Glu-Val-Cit (peptide 3) and β-homo-Glu(EG₆)-Val-Cit (peptide 1) tripeptides were 62.6% and 61.5%, respectively. In comparison, the exatecan release rate from the linking arm with the Val-Cit dipeptide was 34.3%. Thus, the linking arms carrying β-homo-Glu-Val-Cit (peptide 3) and 03-homo-Glu(EG₆)-Val-Cit (peptide 1) tripeptides exhibited more efficient cleavage by CatB than those with the Val-Cit dipeptide.

TABLE 3

| | | % of drug |
|---|---|---|
| | | released after |
| | | HNE digestion |
| Name | Structure | for 48 hr |
| Compound 17 | Lys-PEG linker-β-homo-Glu(EG₆)-Val- | 61.5% |
| | Cit-PABC-EXT | |
| Compound 18 | Lys-PEG linker-β-homo-Glu[(EG₈)₂]- | 6.9% |
| | Val-Cit-PABC-EXT | |
| Compound 19 | Lys-PEG linker-β-homo-Glu-Val-Cit- | 62.9% |
| | PABC-EXT | |
| Compound 21 | Lys-PEG linker-Val-Cit-PABC-EXT | 34.2% |

### Example 28: Drug Release from the Linking Arm of a Drug Bundle Carrying One MMAE Molecule Via Recombinant CatB-Mediated Cleavage

The CatB-mediated drug release assay was conducted as described in the previous example. The results of the MMAE release assay for the linking arm carrying one MMAE molecule were summarized in Table 4. According to the data in Table 4, the release rates of MMAE from linking arms carrying one MMAE molecule with the tripeptides Glu-Val-Cit (peptide 10), Glu-Val-Ala (peptide 11), Glu(EG-Val-Cit (peptide 7), and Glu(EG₆)-Val-Cit (peptide 1) were 98.2, 96.0%, 98.9%, and 21.9%, respectively.

TABLE 4

| | | % of drug |
|---|---|---|
| | | released after |
| | | HNE digestion |
| Name | Structure | for 48 hr |
| Compound 23 | Lys-PEG linker-Glu(EG₃)-Val-Cit- | 98.9% |
| | PABC-MMAE | |
| Compound 24 | Lys-PEG linker-β-homo-Glu-Val-Cit- | 97.9% |
| | PABC-MMAE | |
| Compound 25 | Lys-PEG linker-methylGlu-Val-Cit- | 90.3% |
| | PABC-MMAE | |
| Compound 26 | Lys-PEG linker-Glu-Val-Cit-PABC- | 98.2% |
| | MMAE | |
| Compound 27 | Lys-EG₃-Glu-Val-Ala-PABC-MMAE | 96.0% |

Additionally, the MMAE release rates from linking arms carrying one MMN/AE molecule with β-homo-Glu-Val-Cit or methylGlu-Val-Cit tripeptides were similar to those with Glu-Val-Cit (peptide 9), Glu-Val-Ala (peptide 10), and Glu(EG₃)Val-Cit (peptide 7) tripeptides.

These results suggest that the β-homo-Glu at the P3 position of the P3-P2-P1 tripeptide in the linking arm effectively facilitates cleavage by CatB.

### Example 29: Drug Release from a Dual Drug Bundle Carrying One MMAE Molecule and One Exatecan Molecule Via Recombinant HNE Cleavage

For the DBCO-containing dual drug bundle (compound 36), in which one linking arm [β-homo-Glu(EG₆)-Val-Lys(EG₈)] carries an MMAE molecule and the other linking arm [β-homo-Glu(EG₆)-Val-Cit]carries an exatecan molecule, an HNE-mediated cleavage assay was performed as described in a previous example. The results showed that no free drug release was detected by reverse-phase HPLC.

The finding strongly suggests that the additional EGₛ modification in the Lys backbone at the P3 position of the linking arm [β-homo-Glu(EG₆)-Val-Lys(EG₈)] may further directly or indirectly hinder HNE-mediated cleavage of both the MMAE and exatecan molecules within the bundle. Consequently, the DBCO-containing MMAE/exatecan dual drug bundle exhibits enhanced resistance to HNE.

### Example 30: Solubility Test of Compound 17 and Compound 21

To evaluate the effect of the PEGylated side chain of the β-homo-Glu residue on improving solubility, compound 17, which contains the β-homo-Glu(EG₆)-Val-Cit tripeptide, was compared with compound 21 containing the Val-Cit dipeptide. Briefly, 2 mg of compound 17 and compound 21 powders were dissolved separately in 40 μL of ddH₂O to prepare 50 mg/mL working solutions, which were vortexed for 5 minutes. These 50 mg/mL solutions were then serially diluted to four additional concentrations (10, 2, 0.4, 0.08 mg/mL) by mixing each time at a 1:4 ratio with ddH₂O. All five working solutions were centrifuged at 15,000 g for 10 minutes, and the supernatants were transferred to 1.5 mL Eppendorf tubes. From each solution, 1 μL was taken and mixed with 9 μL of 50% acetonitrile to prepare samples for analysis.

The prepared samples were analyzed using reverse-phase high-performance liquid chromatography (RP-HPLC) in gradient mode. The mobile phase consisted of A (0.1% v/v TFA in water) and B (0.1% v/v TFA in acetonitrile) with a C18 HPLC Column (4.6 mm×25 cm, 5 m), column temperature of 25° C., UV detection at 220 nm, an injection volume of 10 L, and a flow rate of 1 mL/min. The gradient elution was programmed as follows: equilibration from 0 to 8 minutes with 100% mobile phase A, a linear gradient from 8 to 28 minutes with mobile phase B increasing from 0% to 100%, an isocratic hold from 28 to 38 minutes at 100% mobile phase B, followed by re-equilibration from 38 to 40 minutes by returning mobile phase A to 100%. The system was then maintained at 100% mobile phase A from 40 to 48 minutes to re-equilibrate the column.

Data were analyzed using software to calculate R-squared values from the peak areas of each sample. The results showed that compound 17, containing the β-homo-Glu(EG₆)-Val-Cit (peptide 1) tripeptide, dissolved easily and reached a final concentration of 10 mg/mL. In contrast, under the same conditions, precipitation was observed when preparing the 10 mg/mL solution of compound 21, which contains the Val-Cit dipeptide. These results suggest that compound 17 with the β-homo-Glu(EG₆)-Val-Cit (peptide 1) tripeptide has better solubility than compound 21.

### Example 31: Discussion of the Linking Arms of a Drug Bundle Containing β-Homo-Glu or methylGlu Residue with Distinct Effects for Two Different Enzymes

The results from the preceding examples demonstrated that the present peptide linkers were highly resistant to NE degradation while carrying either the MMAE or exatecan molecule.

When the linking arm carries the MMAE molecule, linking arms with β-homo-Glu and methylGlu residues, such as β-homo-Glu-Val-Cit (peptide 3) and methylGlu-Val-Cit (peptide 8), exhibit dual properties: high resistance to HNE enzyme and efficient cleavage by CatB enzyme. Compared to the linking arms with Glu-Val-Cit tripeptide (peptide 9) carrying one MMAE molecule, the β-homo-Glu-Val-Cit tripeptide (peptide 3) exhibited significantly higher resistance to HNE (41.3% vs 0%) and a comparable cleavage rate by CatB (98.2% vs 97.7%). Similarly, the methylGlu-Val-Cit tripeptide demonstrated increased resistance to HNE (41.3% vs 0%) and a similar cleavage rate by CatB (98.2% vs 90.3%).

When the linking arm carries the exatecan molecule, linking arms with β-homo-Glu or β-homo-Glu[EG₆] also exhibit dual properties: resistance to HNE and efficient cleavage by CatB. Compared to the Val-Cit tripeptide linking arms carrying one exatecan molecule, the β-homo-Glu-Val-Cit tripeptide (peptide 3) exhibited greater resistance to HNE (0.4% vs 5.56%) and more efficient cleavage by CatB (62.59% vs 34.25%). Similarly, the β-homo-Glu(EG₆)-Val-Cit tripeptide (peptide 1) exhibited increased resistance to HNE (1.36% vs 5.56%) and a higher cleavage rate by CatB (61.52% vs 34.25%).

Whether carrying MMAE or exatecan, peptides containing β-homo-Glu residues consistently exhibit resistance to HNE while maintaining efficient cleavage by CatB. Additionally, this β-homo-Glu-based tripeptide demonstrates a dual effect, resisting neutrophil elastase while maintaining a high cleavage rate by CatB.

Furthermore, in both cases—(1) the insertion of β-homo-Glu (with or without PEGylation) when carrying the exatecan molecule, and (2) the replacement of Glu with 3-homo-Glu when carrying the MMAE molecule—there was a drastic reduction in cleavage by HNE. A similar effect was also observed when Glu is replaced with methylGlu in the case of MMAE. This strongly suggested that the additional —CH₂— group in the backbone of the β-homo-Glu or methylGlu residue at the P3 position makes it a less suitable substrate for HNE.

### Example 32: Analyses of the Interactions Between β-Homo-Glu-Val-Cit Tripeptide and HNE Peptide

In order to generate the peptide candidates resistant to HNE, molecular dynamics (MD) simulations were conducted in this example. Initially, the 1.84 Å crystal structure of HNE was used to create models of the enzyme bound to Ala-β-homo-Glu-Val-Cit, which corresponds to the P4-P3-P2-P1 position. The protonation states of ionizable side chains at pH 7 were determined using PROPKA3: the His were kept neutral, Arg/Lys were positively charged, and Asp/Glu were negatively charged. The HBUILD module of the CHARMM program in combination with the CHARMM36 force field was used to add the missing hydrogen atoms. The resulting protein-peptide complex was inserted into an orthorhombic box of TIP3P water whose dimensions were set to ensure a distance of at least 12 Å between the complex and the border of the box, resulting in a size of 64×72×63 Å. Water molecules falling within 2.8 Å of any protein heavy atom were removed. Water molecule located further than 4.5 Å from the complex were replaced by either Na⁺ or Cl⁻ ions in order to get neutral system with an ionic strength of 0.15M.

For the simulation protocol, MD simulations were performed at physiological pH at a temperature of 298.15 K and 1 atm pressure using NAMD2.12. All bonds to hydrogen atoms were constrained by the SHAKE algorithm. Long-range electrostatic forces were treated using the particle mesh Ewald method with a grid spacing of 1 Å and a nonbond cutoff of 12 Å. The nonbonded interactions were updated every 1 fs. The solvated protein-peptide complex was submitted to a 10,000 steps minimization of conjugated gradient, followed by an equilibration of the solvent for 50 ps with a time step of 1 fs. This was followed by 100 ps rounds of equilibration during which the restraints on the backbone and side chain atoms were progressively removed, using a time step of 2 fs. The stability of the system was assessed by measuring the root-mean-square-deviation (RMSD) of the backbone atoms compared to the starting structure. The 10,000 conformations between 10 and 20 ns were used for computing the binding free energy of the substrates to HNE.

Then, hydrogen bonds were analyzed using the CHARMM program with an acceptor to hydrogen distance of ≤2.4 Å and a donor-hydrogen-acceptor angle of ≥130°. Occupancy is defined as the proportion of time during which the hydrogen bond exists over every replicate simulation of each complex.

The ΔGsolvelec contribution of the binding free energy was estimated by finite-difference solution of the linearized Poisson-Boltzmann equation implemented in the APBS program. Since a free energy decomposition was performed for each replicate of the two systems, two sets of 20 decompositions were compared. This resulted in a total of 400 ΔΔG values for each residue of the complex in which a positive value was indicative of a loss of binding.

The results of the MD simulation confirmed that the presence of a β-homo-Glu in P2 weakens the interactions between the substrate and the enzyme (data not shown). The substitution of P3 (Glu) by P3 (β-homo-Glu) mainly induced a decrease in the stability of four hydrogen bonds mediated between the backbone of P4 (Ala) and P2 (Val) and the enzyme. The most significant change came from the two hydrogen bonds between P4 (Ala) and the backbone of Val193 of the HNE. The occupancy of these two hydrogen bonds was 70% and 61% after insertion of the extra —CH₂— in the backbone of the P3 residue, while they were highly stable in the reference system (96 and 98%, respectively). In agreement with this observation, the free energy decomposition indicated a loss of binding for P3 (β-homo-Glu), Phe192 and Val193 of HNE. Over 90% of the 400 ΔΔGs computed for these three residues were positive. The decrease of occupancy for the hydrogen bonds of P2 (Val) with Ser191 of the HNE (from 45 to 33%) and Ser176 (from 97 to 89%) was smaller and wasn't associated with a sizeable change in binding energy for the residues involved.

The analyses suggested that, compared to the Glu-Val-Cit tripeptide, the presence of 3-homo-Glu at the P3 position effectively weakened the interactions between the substrate and the enzyme. This finding provided the structural basis for the role of the β-homo-Glu residue in making the tripeptide resistant to HNE.

### Example 33: Preparation of an ADC Conjugated with Multiple DBCO-Containing MMAF/Exatecan Dual Drug Bundles (4 MMAF+4 Exatecan Per Antibody)

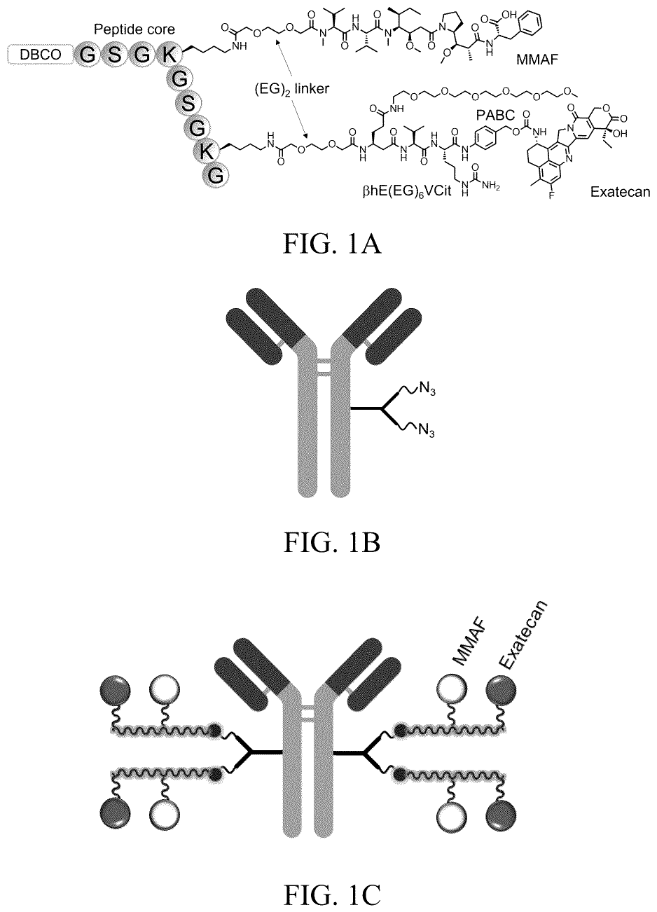

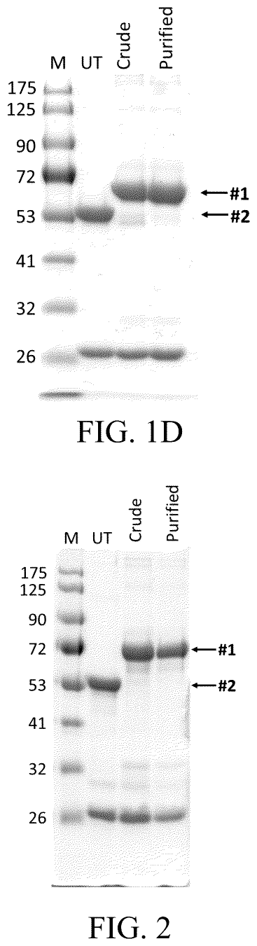

In this example, a molecular construct was prepared in which four DBCO-containing MMAF/exatecan dual-drug bundles were conjugated to the azido-modified glycans on the antibody. The conjugated drug bundle is comprised a peptide core derived from peptide 13, with one linking arm (β-homo-Glu(EG₆)-Val-Cit) carrying an exatecan molecule and another linking arm (EG₂) carrying an MMAF molecule (FIG. 1A). The antibody displayed branched glycans, each containing two azido (N₃) groups (FIG. 1i). A schematic diagram of this construct is shown in FIG. 1C.

Briefly, the antibody was glycoengineered at Asn297 to display branched glycans with a total of four azido groups. Click conjugation of four MMAF/exatecan dual-drug bundles to these azido-modified glycans generated a dual-drug ADC with a defined drug-to-antibody ratio (DAR) of 8. In the schematic, each black circle indicates an azido-DBCO linkage.

For the conjugation, DBCO-containing drug bundles—each consisting of one MMAF molecule and one exatecan molecule—were reacted with azido-modified anti-HER2 antibody (trastuzumab). The azido-modified trastuzumab was concentrated to 10-15 mg/mL and prepared in conjugation buffer (50 mM Tris-HCl, 20-30% sucrose, 10 mM CHAPS, pH 7.0). Then, the modified antibody (1 equiv.) was incubated with DBCO-containing MMAF/exatecan dual drug bundles (6 equiv., 20 mM stock in DMSO) at room temperature under stirring (600 rpm). Different time point (3 hours, 6 hours, and overnight) samples were collected and analyzed by reducing SDS-PAGE.

FIG. 1D depicted the result of the SDS-PAGE analysis of the antibody carrying drug bundles, which comprised linking arms with β-homo-Glu-Val-Cit tripeptide to carry exatecan and MMAF molecules. As indicated in FIG. 1D, this antibody conjugated with four DBCO-containing drug bundle had a molecular weight of about 160 daltons (lane 3 (crude) and 4 (purified), indicated by arrow), somewhat larger than the expected size. The unconjugated antibody was in lane 2. According to the data of FIG. 1D, the yield of the conjugation of DBCO-containing drug bundles to the antibody can reach to 90-95%. Lane 1 is the protein marker (M).

To remove unconjugated and other impurity, hydrophobic interaction column (HIC) purification was applied. In brief, all conjugates were exchanged to buffer A (50 mM NaH₂PO₄, pH 7.0) and applied to a pre-equilibrated HiScreen™ Phenyl HP column. Two washing steps were conducted with 0 and 28% of buffer B (20% IPA) for 5 and 7 column volumes (CV), respectively. The desired ADCs were eluted with 95% buffer B for 5 CV at a flow rate of 1.0 mL/min. Extra 5 CV of 100% buffer B was used to wash out tightly bound proteins. After HIC purification, the ADC was obtained.

The pure product, the anti-HER2 antibody carrying drug bundles, was collected. Lane 2, Lane 3, and Lane 4 depicted in FIG. 1D respectively correspond to unconjugated antibody (labeled as “#2”), the antibody conjugated with drug bundles in the reaction mixture, and the purified antibody conjugated with drug bundles (labeled as “#1”).

### Example 34: Preparation of an ADC Conjugated with Multiple DBCO-Containing MMAE/Exatecan Drug Bundles (4 MMAE+4 Exatecan Per Antibody)

The anti-HER2 ADC having multiple DBCO-containing drug bundles were generated via SPAAC click reaction as described in the earlier Example. The conjugated drug bundle is comprised a peptide core derived from peptide 14, with one linking arm (β-homo-Glu(EG₆)-Val-Lys(EG₈)) carrying an MMAE molecule and another linking arm (β-homo-Glu(EG₆)-Val-Cit) carrying an exatecan molecule. In this example, the DBCO-containing dual drug bundle with one exatecan molecule and one MMAE molecule were conjugated with the azido-modified anti-HER2 antibody.

FIG. 2 depicts the result of the SDS-PAGE analysis of the present antibody carrying DBCO-containing MMAE/exatecan drug bundles, which comprised linking arms with 3-homo-Glu-Val-Cit tripeptide to carry four exatecan and four MMAE molecules. As indicated in FIG. 2, this antibody conjugated with DBCO-containing drug bundle had a molecular weight of about 160 daltons (lane 3 and lane 4, indicated by arrow), somewhat larger than the expected size. Lane 2 (UT), lane 3 (crude), and lane 4 (purified) in FIG. 2 respectively correspond to unconjugated antibody (labeled as “#2”), the antibody conjugated with drug bundles in the reaction mixture, and the purified antibody conjugated with drug bundles (labeled as “#1”). As depicted in FIG. 2, the yield of the conjugation of DBCO-containing drug bundles to the present antibody can reach to 90-95%.

### Example 35: ELISA Analysis of an Anti-HER2 Antibody Conjugated with DBCO-Containing Dual Drug Bundles

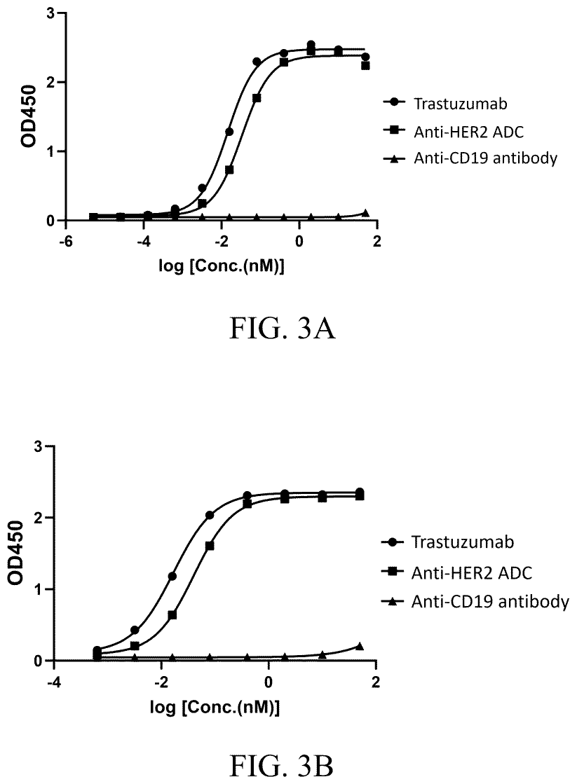

To examine the binding ability of anti-HER2 antibody carrying DBCO-containing dual drug bundles, ELISA assay was performed in this example. ELISA plates were coated with human HER2 protein. Trastuzumab was used as a positive control and the anti-CD19 antibody was used as a negative control. The ELISA results were depicted in FIGS. 3A and 3B.

According to the data of FIGS. 3A and 3B, both of the antibody carrying DBCO-containing MMAE/extatecan dual drug bundles (FIG. 3A, EC₅₀ for trastuzumab and anti-HER2 ADC were respectively 0.014 nM and 0.033 nM, and the level of anti-CD19 Ab was undetectable) and the antibody carrying DBCO-containing MMAF/extatecan dual drug bundles (FIG. 3B, EC₅₀ for trastuzumab and anti-HER2 ADC were respectively 0.017 nM and 0.040 nM, and the level of anti-CD19 Ab was undetectable) exhibited significant binding activity toward human HER2 antigen.

### Example 36: Stable Linkage of MMAF and Exatecan Cytotoxic Drug to the Anti-HER2 Antibody in Human Plasma

The stability of the linkage between MMAF and exatecan cytotoxic drug to the anti-HER2 antibody was evaluated by incubating the ADC in human plasma at 37° C. for 28 days. The release of drug from the anti-HER2 antibody carrying DBCO-containing MMAF/exatecan dual drug bundles in plasma was assessed by incubating samples (10 g anti-HER2 antibody-MMAF/exatecan dual drug bundles in 25 μL of human plasma) at 37° C. for 0, 1, 4, 7, 14, 21, and 28 days.

Payload release was monitored using HPLC, while total and conjugated antibody species was quantified using ELISA.

For HPLC analysis, Samples were stored at −20° C. before analysis. Reactions were quenched with 41.6 μL acetonitrile. The samples were then vortexed, centrifuged at 15,000 g for 15 mins, then incubated for 3 hours at room temperature to evaporate acetonitrile. Released drug was quantified using a Shimadzu Nexera-I LC 2040C system equipped with a Discovery® BIO Wide Pore C18 HPLC Column (5 μm, 4.6×250 mm, Supelco), maintained at 25° C. A 120 μL injection volume was used with UV detection at 220 nm. To separate and quantify released drug, a linear gradient was applied over 30 mins from 0 to 100% acetonitrile in 0.1% TFA and water at 1 mL/min.

In the ELISA analysis, total antibody (regardless of drug loading) levels were measured using an anti-human IgG-Fc-HRP-conjugated antibody. Drug-conjugated species were detected using anti-MMAF and anti-exatecan antibodies.

HPLC analysis showed no detectable release of free MMAF or exatecan during the 28-day incubation period (data not sown), indicating excellent plasma stability and minimal premature payload release. ELISA measurements revealed that the half-life of drug-conjugated trastuzumab (˜11.1-11.5 days) closely matched that of total antibody of the anti-HER2 antibody conjugated with DBCO-containing MMAF/exatecan dual drug bundles (10.6 days) (FIG. 4). These findings confirm that the dual-drug bundles remained stably attached, despite the high DAR of 8.

### Example 37: Half-Life of an Anti-HER2 Antibody Conjugated with DBCO-Containing MMAF/Exatecan Dual Drug Bundles (4 MMAF+4 Exatecan Per Antibody) in B6 Mouse Model

The in vivo half-life of anti-HER2 antibody carrying DBCO-containing MMAF/exatecan dual drug bundles (also named as anti-HER2 ADC-4×MMAF+4EXT) was measured in C57BL/6 (B6) mice following intravenous (i.v.) administration. Serum samples were analyzed using the ELISA method, employing three detection antibodies: anti-IgG.Fc, anti-MMAF, and anti-exatecan antibodies. These detection antibodies were used to measure the half-lives of both the antibody component (referred to as the “total antibody”) and the conjugated forms of the antibody. 8- to 10-week-old B6 mice were divided into groups of five mice each, and given an intravenous bolus injection at a dose of 4 mg/kg.

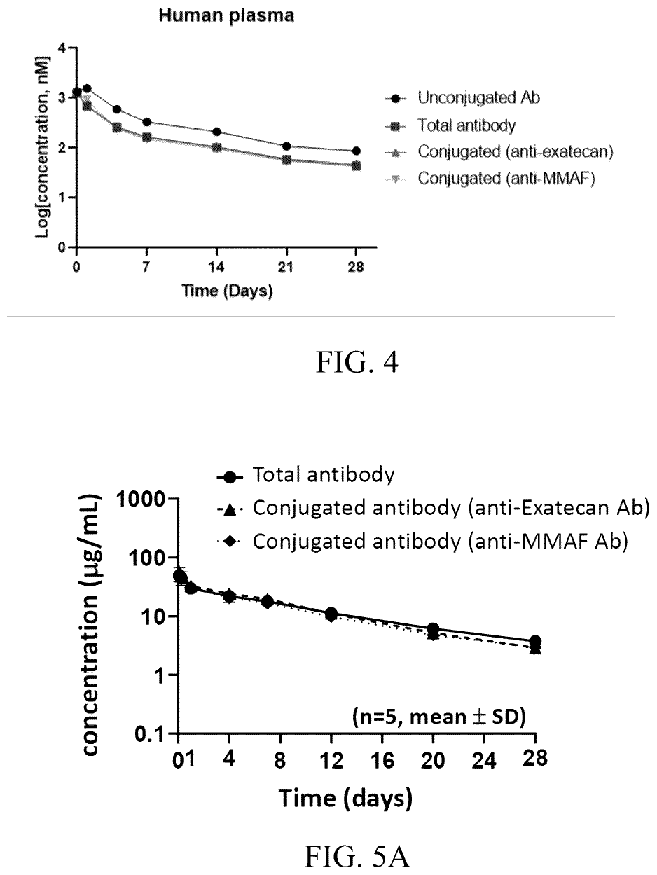

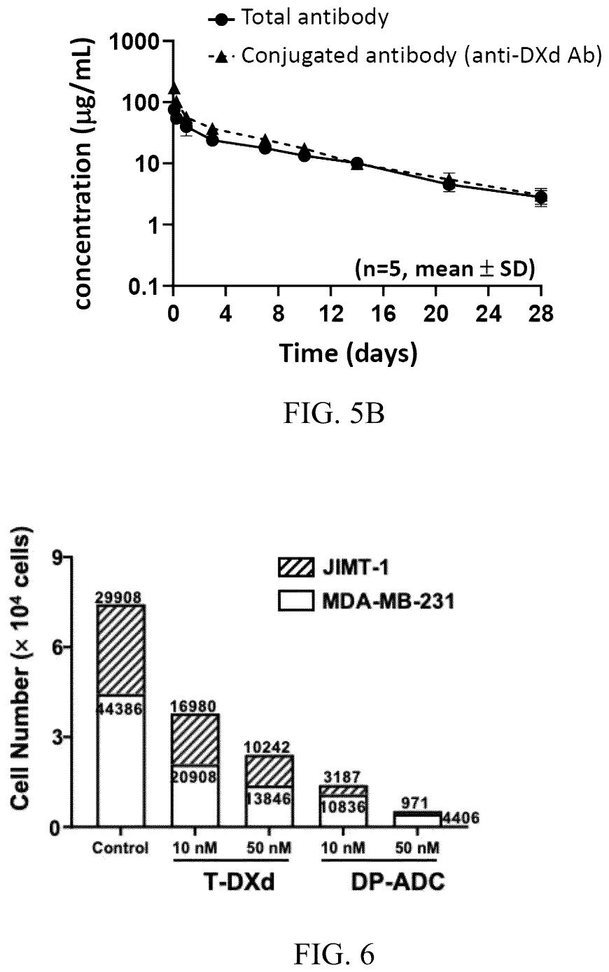

The results depicted in FIG. 5 indicated that the half-life of the antibody component of the anti-HER2 antibody carrying DBCO-containing MMAF/exatecan dual drug bundles was 9.12 hours. The half-lives of the conjugates, measured using the anti-MMAF and anti-exatecan antibodies, were approximately 7.8 and 7.7 hours, respectively (FIG. 5A, Table 5). In comparison, the half-life of trastuzumab deruxtecan's antibody component was 6.23 hours, and the half-life of its conjugates was 5.36 hours, as measured using the anti-IgG.Fc and anti-Dxd antibodies, respectively (FIG. 5B, Table 6).

TABLE 5

| Anti-HER2 ADC-4x MMAF + 4x EXT | T_{1/2} |
|---|---|
| Total antibody | 9.12 |
| Conjugated antibody (anti-Exatecan Ab) | 7.88 |
| Conjugated antibody (anti-MMAF Ab) | 7.77 |

TABLE 6

| Trastuzumab deruxtean | T_{1/2} |
|---|---|
| Total antibody | 6.23 |
| Conjugated antibody (anti-Dxd Ab) | 5.36 |

These findings indicate that the anti-HER2 antibody carrying DBCO-containing MMAF/exatecan dual drug bundles has a longer half-life than trastuzumab deruxtecan.

In this study, no free exatecan was detected in plasma by liquid chromatography-tandem mass spectrometry (LC-MS/MS) up to 7 days post-injection, confirming the absence of premature payload release. Together, these results demonstrate that anti-HER2 antibody conjugated with DBCO-containing MMAF/exatecan dual drug bundles retains both payloads in circulation, despite its DAR of 8 and dual-payload configuration.

### Example 38: In Vitro Efficacy of an Anti-HER2 Antibody Conjugated with DBCO-Containing MMAF/Exatecan Dual Drug Bundles (4 MMAF+4 Exatecan Per Antibody)

The cytotoxic effects of the ADCs were evaluated in both HER2-positive (SK-BR3, NCI-N87, JIMT-1) and HER2-negative (MDA-MB-231) cell lines. Cells were seeded in a 96-well plate at a density of 3000-4000 cells per well (depending on the cell line growth rate) and cocultured with fresh medium containing concentrations of the anti-HER2 antibody carrying DBCO-containing MMAF/exatecan dual drug bundles ranging from 0.128 pM to 10 nM for 5 days at 37° C. Cell viability was then determined using the alamarBlue™ cell viability reagent (Thermo Fisher Scientific). AlamarBlue™ (10 μL) was added to each well containing 100 μL medium, and the plates were incubated at 37° C. for 1 h. Fluorescence was measured using a microplate reader with excitation at 560 nm and emission at 590 nm.

Cytotoxicity of anti-HER2 antibody carrying DBCO-containing MMAF/exatecan dual drug bundles (also shown as Dual-payload ADC) and trastuzumab deruxtecan (also shown as T-DXd) was compared in cell lines with varying HER2 expression: HER2⁺⁺⁺-high (SK-BR-3, NCI-N87), HER2⁺⁺-medium (JIMT-1), HER2⁺-low (MDA-MB-361), and HER²⁻ (MDA-MB-231, control). After incubating the various cells with different drug concentrations for 5 days at 37° C., the percentage of cell viability and maximum cell killing were determined.

The results showed that Anti-HER2 antibody carrying DBCO-containing MMN/AF/exatecan dual drug bundles (referred to as the dual-payload ADC) exhibited significantly greater potency than T-DXd with lower IC₅₀ values and greater maximum cell killing in all HER2-expressing cell lines (Table 7). Notably, in trastuzumab-resistant JIMT-1 cells where MMAF has known activity, anti-HER2 antibody carrying DBCO-containing MMAF/exatecan dual drug bundles retained efficacy, but T-DXd showed no detectable activity. Furthermore, anti-HER2 antibody carrying DBCO-containing MMAF/exatecan dual drug bundles was 20-fold more potent than T-DXd in HER2⁺-low MDA-MVB-361 cells. Neither ADC showed detectable activity in HER2⁻ cells.

TABLE 7

| | Cell Type | | | |
|---|---|---|---|---|
| | SK-BR-3 | NCI-N87 | JIMT-1 | MDA-MB-361 |
| Name of ADC | (HER2⁺⁺⁺) | (HER2⁺⁺⁺) | (HER2⁺⁺⁺) | (HER2⁺) |
| | IC₅₀ (nM) | | | |
| Dual-payload | 0.005 ± 0.002 | 0.036 ± 0.004 | 0.112 ± 0.028 | 0.013 ± 0.0005 |
| ADC | | | | |
| T-Dxd | 0.0084 ± 0.024 | 0.204 ± 0.045 | N.D.^{a} | 0.264 ± 0.058 |

Maximum percentage of cell killing

| | | | | |
|---|---|---|---|---|
| Dual-payload | 97.3 | 82.2 | 89.5 | 89.7 |
| ADC | | | | |
| T-Dxd | 90.8 | 60.5 | N.D.^{a} | 44.7 |

N.D.^{a} = Not detected (no inhibition observed in trastzumab-resistant or HER2⁻ cells)

### Example 39: Synergistic Anti-Tumor Activity of an Anti-HER2 Antibody Conjugated with DBCO-Containing MMAF/Exatecan Dual Drug Bundles (4 MMAF+4 Exatecan Per Antibody)

To evaluate the bystander killing activity of the dual-payload anti-HER2 ADC and compare it to T-DXd, HER2⁺⁺ JIMT-1 cells (3.25×10⁴ per well) and HER2⁻ MDA-MB-231 cells (1.75×10⁴ per well) were co-seeded at a 6.5:3.5 ratio in 24-well plates (250 al/well). After overnight incubation, the supernatant was removed, and each antibody-drug conjugate diluent (250 μl/well) was added to achieve a final concentration of 10 nM or 50 nM (total volume: 500 μl/well). At 5 days post-treatment, cells were harvested and analyzed by flow cytometry. In order to distinguish JIMT-1 cells from MDA-MB-231 cells, the latter were engineered to express green fluorescence protein (GFP). GFP+ MDA-MB-231 cells were created by transfecting MDA-MB-231 cells with a lentiviral vector containing the GFP gene at a multiplicity of infection factor (MOI) of 50. Cells were supplemented with cationic polymer polybrene at 10 μg/mL to facilitate subsequent virus penetration. After overnight incubation, the cells were selected by puromycin (1 μg/mL) and sorted using FACS melody (BD). The resulting GFP⁺ MDA-MB-231 cells could then be distinguished using GFP intensity. After excluding dead cells using a live/dead exclusion dye, viable cells were counted using a cell counter.

In this study, bystander killing was evaluated using a 5-day co-culture assay with HER2⁺⁺ JIMT-1 and HER2− MDA-MB-231 cells. The results in FIG. 6 showed that, compared to T-DXd, the dual-payload anti-HER2 ADC (referred to as “DP-ADC” in FIG. 6) more effectively killed HER2⁺⁺ JIMT-1 cells, leaving 5-10 times less viable cells at both tested concentrations (10 and 50 nM). The dual-payload ADC also killed more HER2− MDA-MB-231 cells than T-DXd, leaving 2-3 times less viable cells at both tested concentrations. However, the difference between the dual-payload ADC and T-DXd in HER2− MDA-MB-231 cell killing was less dramatic than that observed for direct killing of HER2⁺⁺ JIMT-1 cells.

### Example 40: Establishment of Xenograft HER2-Positive JIMT-1 Breast Tumor Mouse Model

JIMT-1 (5×10⁶) in DMEM (1×) were mixed with 50% (v/v) extracellular matrix gel (Matrigel, Corning) and injected into the flanks of SCID mice to generate intradermal tumors. Tumor size and body weight were recorded every 3 to 4 days. Once average tumor volume reached 140±10 mm3 (11-17 days post-transplantation), mice were selected for treatment. Tumor volumes were measured using calipers and calculated as (length×width×width)/2. At the end of the experiments, tumors were collected and weighed.

### Example 41: In Vivo Efficacy Study of an Anti-HER2 Antibody Conjugated with DBCO-Containing MMAF/Exatecan Dual Drug Bundles (4 MMAF+4 Exatecan Per Antibody)

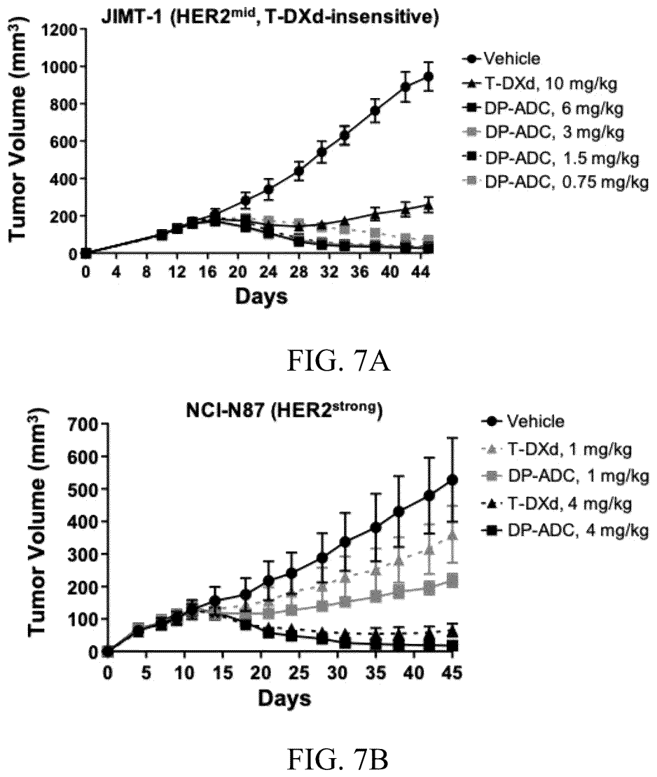

In the cell-derived xenograft (CDX) model using trastuzumab-resistant JIMT-1 tumors with medium HER2 expression, mice received four weekly intravenous doses (Q7D×4) of vehicle, T-DXd (10 mg/kg), or the dual-payload ADC (referred to as the DP-ADC) (0.75, 1.5, 3, or 6 mg/kg), and tumor volumes were measured twice weekly, over 63 days. Both anti-HER2 antibody carrying DBCO-containing MMAF/exatecan dual drug bundles and T-DXd initially suppressed tumor growth (FIG. 7A). However, tumors in the T-DXd-treated mice regrew after the third dose, indicating waning efficacy. In contrast, the dual-payload ADC induced dose-dependent and sustained tumor regression at doses≥1.5 mg/kg, with maximal reduction observed between the second and fourth doses. By day 52, tumor volumes in the dual-payload ADC-treated mice (1.5-6 mg/kg) were ˜10-fold smaller than those in T-DXd-treated mice, despite T-DXd being given at a higher dose (10 mg/kg). Although tumor responses at 0.75 mg/kg was variable, it still showed better efficacy than T-DXd at 10 mg/kg. These results confirm the dual-payload ADC's ability to overcome resistance mechanisms observed with T-DXd, consistent with in vitro cytotoxicity results (Table 7), where the dual-payload ADC retained potent activity in trastuzumab-resistant JIMT-1 cells, while T-DXd showed no detectable effect.

In a second CDX model using NCI-N87 tumors with high HER2 expression, mice received a single intravenous dose of vehicle, T-DXd (1 or 4 mg/kg), or the dual-payload ADC (0.25-4 mg/kg), and tumor volume was measured twice weekly. Unlike the JIMT-1 CDX model, a single 4 mg/kg dose of T-DXd could already induce tumor regression (FIG. 7B). Although both the dual-payload ADC and T-DXd at 4 mg/kg induced tumor regression, the endpoint tumor weights were lower in the dual-payload ADC group, consistent with in vitro cytotoxicity results.

### Example 42: Tolerability Study of an Anti-HER2 Antibody Conjugated with DBCO-Containing MMAF/Exatecan Dual Drug Bundles (4 MMAF+4 Exatecan Per Antibody) in ICR Mouse Model

To evaluate the tolerability of anti-HER2 antibody carrying DBCO-containing MMAF/exatecan dual drug bundles, healthy male ICR mice were administered a single intravenous dose of an anti-HER2 antibody conjugated with DBCO-containing MMAF/exatecan dual drug bundles. Animals were randomized into three groups (n=5 per group) and received either 40 mg/kg, 60 mg/kg, or PBS as control.

Body weight, clinical signs, and food intake were monitored on Days −7, −3, and −1 prior to sacrifice. On Day 7 post-dosing, all surviving animals were euthanized for blood collection (hematology and serum biochemistry), necropsy, and organ harvest with weight measurement. Ophthalmologic examination and detailed clinical observations were also conducted during the study period.

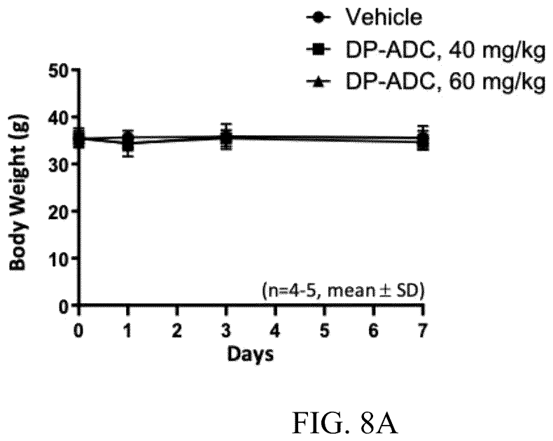

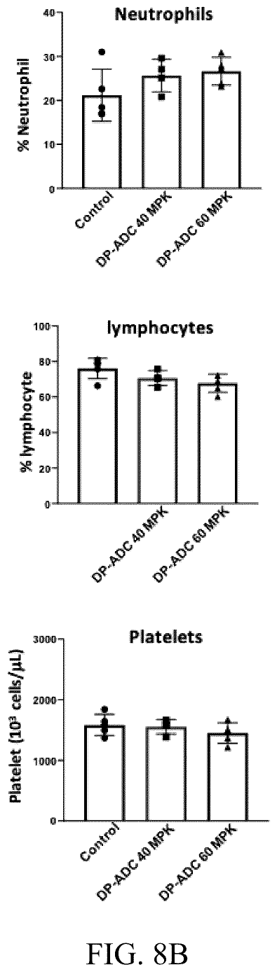

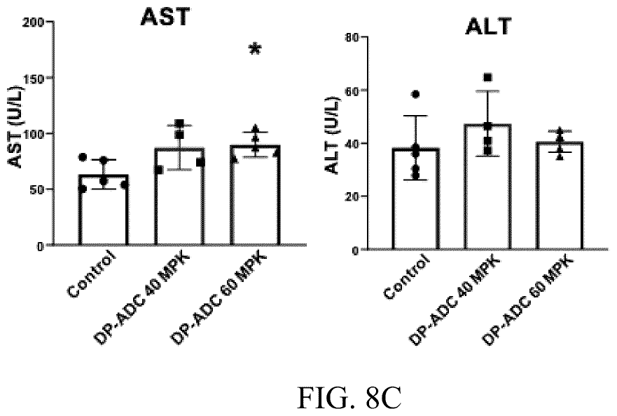

The anti-HER2 antibody carrying DBCO-containing MMAF/exatecan dual drug bundles (referred to as the DP-ADC) was well tolerated at both dose levels. No mortality occurred, and no significant body weight loss (>5%) (FIG. 8A) or reduction in food consumption was observed compared with controls. Clinical and ophthalmologic examinations revealed no abnormal signs. Hematology results indicated that neutrophil, lymphocyte, and platelet counts remained within normal ranges (FIG. 8B). Serum biochemistry showed that aspartate aminotransferase (AST) and alanine aminotransferase (ALT) levels were also within normal ranges (FIG. 8C).

Gross pathology did not reveal treatment-related abnormalities. One animal in the 40 mg/kg group was excluded from the analysis due to unrelated individual variability, with no issues detected in the remaining animals. Organ weights were unremarkable and did not suggest compound-related toxicity.

In summary, the tolerability study showed that administration of anti-HER2 antibody carrying DBCO-containing MMAF/exatecan dual drug bundles at single intravenous doses of up to 60 mg/kg in ICR mice was well tolerated, with no adverse clinical findings, hematological or biochemical abnormalities, or gross pathological changes. These results indicate that the maximum tolerated dose (MTD) in mice exceeds 60 mg/kg, supporting further preclinical development and evaluation of anti-HER2 antibody carrying DBCO-containing MMAF/exatecan dual drug bundles.

It will be understood that the above description of embodiments is given by way of example only and that various modifications may be made by those with ordinary skill in the art. The above specification, examples, and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A compound having the structure of formula (I), 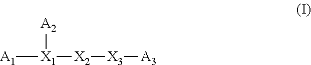
wherein,
X₁ is an acidic β-homo-amino acid, or an acidic α-methyl-amino acid;
X₂ is valine (V) or leucine (L);
X₃ is citrulline, alanine (A) or lysine (L); and
A₁, A₂ and A₃ are independently nil or a polyethylene glycol (PEG) moiety comprising 1 to 24 repeats of ethylene glycol (EG) unit; and
A₃ is nil when X₃ is not lysine.

2. The compound of claim 1, wherein the acidic amino acid is the β-homo-amino acid is β-homo-glutamic acid or β-homo-aspartic acid; and the α-methyl-amino acid is α-methyl-glutamic acid or α-methyl-aspartic acid.

3. The compound of claim 2, wherein X₁ is β-homo-glutamic acid, X₂ is valine, X₃ is citrulline, A₁ is the PEG moiety comprising 6 repeats of EG unit, and A₂ and A₃ are independently nil.

4. The compound of claim 2, wherein X₁ is β-homo-glutamic acid, X₂ is valine, X₃ is citrulline, A₁ and A₂ are independently the PEG moiety comprising 8 repeats of EG unit, and A₃ is nil.

5. The compound of claim 2, wherein X₁ is β-homo-glutamic acid, X₂ is valine, X₃ is citrulline, and A₁, A₂ and A₃ are independently nil.

6. The compound of claim 2, wherein X₁ is α-methyl-glutamic acid, X₂ is valine, X₃ is citrulline, and A₁, A₂ and A₃ are independently nil.

7. The compound of claim 2, wherein X₁ is β-homo-glutamic acid, X₂ is valine, X₃ is lysine, A₁ is the PEG moiety comprising 6 repeats of EG unit, A₂ is nil, and A₃ is the PEG moiety comprising 8 repeats of EG unit.

8. The compound of claim 2, wherein X₁ is β-homo-glutamic acid, X₂ is valine, X₃ is citrulline, A₁ and A₂ are independently the PEG moiety comprising 12 repeats of EG unit, and A₃ is nil.

9. The compound of claim 8, wherein one of the two PEG moieties further comprises a sarcosine linked to its terminus.

10. A linker unit comprising,
a center core comprising,
1 to 6 lysine (K) residues;
optionally, one or more fillers, wherein any two of the 1-6 K residues are adjacent to each other or are separated by the one or more fillers;
optionally, a terminal spacer, linked to the N-terminus of the first K residue or linked to the C-terminus of the last K residue, wherein each filler and the terminal spacer comprises, independently, (1) 1 to 6 non-K amino acid residues, or (2) a PEGylated amino acid having 1 to 6 repeats of EG unit; and
a conjugating group, linked to the first or last K residue of the center core, or when the terminal spacer is present, the conjugating group is linked to the terminal amino acid residue of the terminal spacer, wherein the conjugating group is selected from the group consisting of azide, alkyne, tetrazine, cyclooctene and cyclooctyne groups;
1 to 6 effector elements; and
1 to 6 linking arms, wherein one terminus of each linking arm is linked to one of the 1-6 K residues of the center core, and the other terminus of each linking arm is linked to one of the effector elements;
wherein,
each linking arm is independently the compound of claim 1 or a PEG moiety comprising 1 to 4 repeats of EG unit; and
at least one of the linking arms is the compound of claim 1.

11. The linker unit of claim 10, wherein the center core comprises a peptide of SEQ ID NOs: 1, 2 or 3.

12. The linker unit of claim 11, further comprising a targeting element linked to the conjugating group of the center core.

13. The linker unit of claim 12, wherein the targeting element is an antibody specific to a tumor-associated antigen (TAA), and the effector element is a cytotoxic drug.

14. The linker unit of claim 13, wherein the TAA specific antibody is an anti-epidermal growth factor receptor (HER) 2 antibody, and the cytotoxic drug is acalabrutinib, exatecan, monomethyl auristatin E (MMAE), monomethyl auristatin F (MMAF), niraparib or vepdegestrant (ARV-471).

15. An antibody-drug conjugate (ADC) comprising an antibody and 2 to 8 linker units independently linked to the antibody, wherein each of the linker units is the linker unit of claim 16.

16. The ADC of claim 15, wherein the antibody is specific to a tumor-associated antigen (TAA), and the effector element is a cytotoxic drug.

17. The ADC of claim 16, wherein the TAA specific antibody is an anti-HER2 antibody, and the cytotoxic drug is exatecan, MMAE or MMAF.

18. A method of treating cancer in a subject comprising administering to the subject an effective amount of linker unit of claim 10 or the ADC of claim 15.

19. The method of claim 18, wherein the subject is a human.
